(19) United States
(12) Patent Application Publication
(10) Pub. No.: US 2010/0286181 A1
(43) Pub. Date: Nov. 11, 2010

(54) PYRROLE DERIVATIVES WITH ANTIBACTERIAL ACTIVITY

(75) Inventors: Gregory Basarab, Waltham, MA (US); Pamela Hill, Waltham, MA (US); Kenneth Gregory Hull, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/377,785

(22) PCT Filed: Aug. 16, 2007
(86) PCT No.: PCT/GB07/03134

§ 371 Date: Feb. 17, 2009

Related U.S. Application Data
(60) Provisional application No. 60/822,747, filed on Aug. 17, 2006.
(60) Provisional application No. 60/822,923, filed on Aug. 18, 2006.
(60) Provisional application No. 60/890,151, filed on Feb. 15, 2007.

(51) Int. Cl. A61K 31/438; C07D 491/113; C07D 417/14; A61K 31/454; A61P 31/04; A61P 13/02; A61P 11/00

(52) U.S. Cl. 514/278; 546/19; 546/209; 514/326; 546/198; 514/321

(57) ABSTRACT

Compounds of formula (I) and their pharmaceutically acceptable salts are described. Processes for their preparation, pharmaceutical compositions containing them, their use as medicaments and their use in the treatment of bacterial infections are also described.

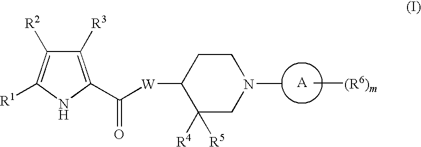

### BACKGROUND OF THE INVENTION

The present invention relates to compounds which demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. In particular this invention relates to compounds useful for the treatment of bacterial infections in warm-blooded animals such as humans, more particularly to the use of these compounds in the manufacture of medicaments for use in the treatment of bacterial infections in warm-blooded animals such as humans.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus aureus (MRSA), methicillin resistant coagulase negative staphylococci (MRCNS), penicillin resistant Streptococcus pneumoniae and multiple resistant Enterococcus faecium.

The preferred clinically effective antibiotic for treatment of last resort of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Consequently, in order to overcome the threat of widespread multi-drug resistant organisms, there is an on-going need to develop new antibiotics, particularly those with either a novel mechanism of action and/or containing new pharmacophoric groups.

Deoxyribonucleic acid (DNA) gyrase is a member of the type II family of topoisomerases that control the topological state of DNA in cells (Champoux, J. J.; 2001. Ann. Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

DNA gyrase is a well-validated target of antibacterials, including the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated primarily by mutations in the target (DNA gyrase and topoisomerase IV) is becoming an increasing problem in several organisms, including S. aureus and Streptococcus pneumoniae (Hooper, D.C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

Synthetic inhibitors that target the B subunit of DNA gyrase and topoisomerase IV are known in the art. For example, coumarin-containing compounds are described in patent application number WO 99/35155,5,6-bicyclic heteroaromatic compounds are described in patent application WO 02/060879, and pyrazole compounds are described in patent application WO 01/52845 (U.S. Pat. No. 6,608,087).

We have discovered a new class of compounds which are useful for inhibiting DNA gyrase and/or topoisomerase IV.

### SUMMARY OF THE INVENTION

Therefore the present invention provides a compound of formula (I):

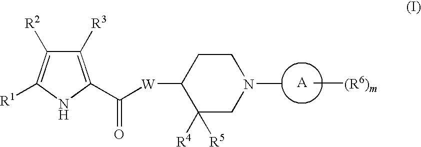

wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;

R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;

W is —O—, —N(R⁷)— or —C(R⁸)(R⁹)—;

Ring A is carbocyclyl or heterocyclyl; wherein if said heterocyclyl contains an —NH— moiety that nitrogen may be optionally substituted by a group selected from R¹⁰;

R⁴ and R⁵ are selected from the following groups: (i) one of R⁴ and R⁵ is hydrogen and the other one is selected from azido, amino or heterocyclyl; or (ii) R⁴ and R⁵ are independently selected from an C₁₋₆alkyl or an C₁₋₆alkoxy group; or (iii) R⁴ and R⁵ together form oxo, R¹¹R¹²N—N═ or R¹³O—N═; or (iv) R⁴ and R⁵ together with the carbon to which they are attached form 3-6 membered carbocyclic or heterocyclic ring wherein said ring may be optionally spiro-fused to a further 3-6 membered carbocyclic or heterocyclic ring; wherein R⁴ and R⁵ in any of groups (i)-(iv) may be optionally substituted on carbon by one or more R¹⁴; and wherein if said heterocyclyl in group (i) or heterocyclic ring in group (iv) contains an —NH— moiety that nitrogen may be optionally substituted by a group selected from R¹⁵;

R⁶ is a substituent on carbon and is selected from azido, halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkenyloxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁶— or heterocyclyl-R¹⁷; wherein R⁶ may be optionally substituted on carbon by one or more R¹⁸; and wherein if said heterocyclyl contains an —NH— moiety that nitrogen may be optionally substituted by a group selected from R¹⁹;

m is 0-4; wherein the values of R⁶ may be the same or different;

R⁷, R⁸ and R⁹ are independently selected from hydrogen or C₁₋₄alkyl;

R¹¹, R¹² and R¹³ are independently selected from hydrogen, C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; or R¹¹ and R¹² together with the nitrogen to which they are attached form a heterocyclic group; wherein R¹¹, R¹² and R¹³ may be independently optionally substituted on carbon by one or more R²⁰; and wherein if said heterocyclyl contains an —NH— moiety that nitrogen may be optionally substituted by a group selected from R²¹;

R¹⁴ and R¹⁸ are independently selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R²²— or heterocyclyl-R²³—; or two R¹⁴ or two R¹⁸ may together form methylene; wherein R¹⁴ and R¹⁸ may be independently optionally substituted on carbon by one or more R²⁴; and wherein if said heterocyclyl contains an —NH— moiety that nitrogen may be optionally substituted by a group selected from R²⁵;

R¹⁰, R¹⁵, R¹⁹, R²¹ and R²⁵ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; wherein R¹⁰, R¹⁵, R¹⁹, R²¹ and R²⁵ may be independently optionally substituted on carbon by one or more R³¹;

R¹⁶, R¹⁷, R²² and R²³ are independently selected from a direct bond, —O—, —N(R²⁶)—, —C(O)—, —N(R²⁷)C(O)—, —C(O)N(R²⁸)—, —S(O)_{P}, —SO₂N(R²⁹)— or —N(R³⁰)SO₂—; wherein R²⁶, R²⁷, R²⁸, R²⁹ and R³⁰ are independently selected from hydrogen or C₁₋₄alkyl and p is 0-2;

R²⁰, R²⁴ and R³¹ are independently selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;
with the proviso that said compound is not:
cis(±)-methyl 2-(3-azido-4-{[(3,4-dichloro-5-methyl-1H-pyrrol-2-yl)carbonyl]amino}piperidin-1-yl)-1,3-thiazole-5-carboxylate; or
(±)-2-(3-azido-4-{[(3,4-dichloro-5-methyl-1H-pyrrol-2-yl)carbonyl]amino}piperidin-1-yl)-1,3-thiazole-5-carboxylic acid.

In a further aspect of the invention there is provided a compound of formula (I) wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;

R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;

W is —O—, —N(R⁷)— or —C(R⁸)(R⁹)—;

Ring A is carbocyclyl or heterocyclyl; wherein if said heterocyclyl contains an —NH— moiety that nitrogen may be optionally substituted by a group selected from R¹⁰;

R⁴ and R⁵ are selected from the following groups: (i) one of R⁴ and R⁵ is hydrogen and the other one is selected from azido or heterocyclyl; or (ii) R⁴ and R⁵ are independently selected from an C₁₋₆alkyl or an C₁₋₆alkoxy group; or (iii) R⁴ and R⁵ together form oxo, R¹¹R¹²N—N═ or R¹³O—N═; or (iv) R⁴ and R⁵ together with the carbon to which they are attached form 3-6 membered carbocyclic or heterocyclic ring; wherein R⁴ and R⁵ in any of groups (i)-(iv) may be optionally substituted on carbon by one or more R¹⁴; and wherein if said heterocyclyl in group (i) or heterocyclic ring in group (iv) contains an —NH— moiety that nitrogen may be optionally substituted by a group selected from R¹⁵;

R⁶ is a substituent on carbon and is selected from azido, halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkenyloxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁶— or heterocyclyl-R¹⁷—; wherein R⁶ may be optionally substituted on carbon by one or more R¹⁸; and wherein if said heterocyclyl contains an —NH— moiety that nitrogen may be optionally substituted by a group selected from R¹⁹;

m is 0-4; wherein the values of R⁶ may be the same or different;

R⁷, R⁸ and R⁹ are independently selected from hydrogen or C₁₋₄alkyl;

R¹¹, R¹² and R¹³ are independently selected from hydrogen, C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; or R¹¹ and R¹² together with the nitrogen to which they are attached form a heterocyclic group; wherein R¹¹, R¹² and R¹³ may be independently optionally substituted on carbon by one or more R²⁰; and wherein if said heterocyclyl contains an —NH— moiety that nitrogen may be optionally substituted by a group selected from R²¹;

R¹⁴ and R¹⁸ are independently selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R²²— or heterocyclyl-R²³—; wherein R¹⁴ and R¹⁸ may be independently optionally substituted on carbon by one or more R²⁴; and wherein if said heterocyclyl contains an —NH— moiety that nitrogen may be optionally substituted by a group selected from R²⁵;

R¹⁰, R¹⁵, R¹⁹, R²¹ and R²⁵ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁶, R¹⁷, R²² and R²³ are independently selected from a direct bond, —O—, —N(R²⁶)—, —C(O)—, —N(R²⁷)C(O)—, —C(O)N(R²⁸)—, —S(O)ₚ—, —SO₂N(R²⁹)— or —N(R³⁰)SO₂—; wherein R²⁶, R²⁷, R²⁸, R²⁹ and R³⁰ are independently selected from hydrogen or C₁₋₄alkyl and p is 0-2;

R²⁰ and R²⁴ are independently selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

In a further aspect of the invention there is provided a compound of formula (I) wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;

R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;

W is —O—, —N(R⁷)— or —C(R⁸)(R⁹)—;

Ring A is carbocyclyl or heterocyclyl; wherein if said heterocyclyl contains an —NH— -moiety that nitrogen may be optionally substituted by a group selected from R¹⁰;

R⁴ and R⁵ are selected from the following groups: (i) one of R⁴ and R⁵ is hydrogen and the other one is selected from azido or heterocyclyl; or (ii) R⁴ and R⁵ are independently selected from an C₁₋₆alkyl or an C₁₋₆alkoxy group; or (iii) R⁴ and R⁵ together form oxo, R¹¹R¹²N—N═ or R¹³O—N═; or (iv) R⁴ and R⁵ together with the carbon to which they are attached form 3-6 membered carbocyclic or heterocyclic ring; wherein R⁴ and R⁵ in any of groups (i)-(iv) may be optionally substituted on carbon by one or more R¹⁴; and wherein if said heterocyclyl in group (i) or heterocyclic ring in group (iv) contains an —NH— moiety that nitrogen may be optionally substituted by a group selected from R¹⁵;

R⁶ is a substituent on carbon and is selected from azido, halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkenyloxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁶— or heterocyclyl-R¹⁷—; wherein R⁶ may be optionally substituted on carbon by one or more R¹⁸; and wherein if said heterocyclyl contains an —NH— moiety that nitrogen may be optionally substituted by a group selected from R¹⁹;

m is 0-4; wherein the values of R⁶ may be the same or different;

R⁷, R⁸ and R⁹ are independently selected from hydrogen or C₁₋₄alkyl;

R¹¹, R¹² and R¹³ are independently selected from hydrogen, C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; or R¹¹ and R¹² together with the nitrogen to which they are attached form a heterocyclic group; wherein R¹¹, R¹² and R¹³ may be independently optionally substituted on carbon by one or more R²⁰; and wherein if said heterocyclyl contains an —NH— moiety that nitrogen may be optionally substituted by a group selected from R²¹;

R¹⁴ and R¹⁸ are independently selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R²²— or heterocyclyl-R²³—; wherein R¹⁴ and R¹⁸ may be independently optionally substituted on carbon by one or more R²⁴; and wherein if said heterocyclyl contains an —NH— moiety that nitrogen may be optionally substituted by a group selected from R²⁵;

R¹⁰, R¹⁵, R¹⁹, R²¹ and R²⁵ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁶, R¹⁷, R²² and R²³ are independently selected from a direct bond, —O—, —N(R²⁶)—, —C(O)—, —N(R²⁷)C(O)—, —C(O)N(R²⁸)—, —S(O)ₚ—, —SO₂N(R²⁹)— or —N(R³⁰)SO₂—; wherein R²⁶, R²⁷, R²⁸, R²⁹ and R³⁰ are independently selected from hydrogen or C₁₋₄alkyl and p is 0-2;

R²⁰ and R²⁴ are independently selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;
with the proviso that said compound is not:
cis(±)-methyl 2-(3-azido-4-{[(3,4-dichloro-5-methyl-1H-pyrrol-2-yl)carbonyl]amino}piperidin-1-yl)-1,3-thiazole-5-carboxylate; or
cis(±)-2-(3-azido-4-{[(3,4-dichloro-5-methyl-1H-pyrrol-2-yl)carbonyl]amino}piperidin-1-yl)-1,3-thiazole-5-carboxylic acid.

In a further aspect of the invention there is provided a compound of formula (I) wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;

R³ is selected from hydrogen, nitro, hydroxy, halo, cyano, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;

W is —O—, —N(R⁷)— or —C(R⁸)(R⁹)—;

Ring A is carbocyclyl or heterocyclyl; wherein if said heterocyclyl contains an —NH— moiety that nitrogen may be optionally substituted by a group selected from R¹⁰;

R⁴ and R⁵ are selected from the following groups: (i) one of R⁴ and R⁵ is hydrogen and the other one is selected from azido, amino or heterocyclyl; or (ii) R⁴ and R⁵ are independently selected from an C₁₋₆alkyl or an C₁₋₆alkoxy group; or (iii) R⁴ and R⁵ together form oxo, R¹¹R¹²N—N═ or R¹³O—N═; or (iv) R⁴ and R⁵ together with the carbon to which they are attached form 3-6 membered carbocyclic or heterocyclic ring wherein said ring may be optionally spiro-fused to a further 3-6 membered carbocyclic or heterocyclic ring; wherein R⁴ and R⁵ in any of groups (i)-(iv) may be optionally substituted on carbon by one or more R¹⁴; and wherein if said heterocyclyl in group (i) or heterocyclic ring in group (iv) contains an —NH— moiety that nitrogen may be optionally substituted by a group selected from R¹⁵;

R⁶ is a substituent on carbon and is selected from azido, halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkenyloxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁶— or heterocyclyl-R¹⁷—; wherein R⁶ may be optionally substituted on carbon by one or more R¹⁸; and wherein if said heterocyclyl contains an —NH— moiety that nitrogen may be optionally substituted by a group selected from R¹⁹;

m is 0-4; wherein the values of R⁶ may be the same or different;

R⁷, R⁸ and R⁹ are independently selected from hydrogen or C₁₋₄alkyl;

R¹¹, R¹² and R¹³ are independently selected from hydrogen, C₁₋₄alkyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; or R¹¹ and R¹² together with the nitrogen to which they are attached form a heterocyclic group; wherein R¹¹, R¹² and R¹³ may be independently optionally substituted on carbon by one or more R²⁰; and wherein if said heterocyclyl contains an —NH— moiety that nitrogen may be optionally substituted by a group selected from R²¹;

R¹⁴ and R¹⁸ are independently selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R²²— or heterocyclyl-R²³—; or two R¹⁴ or two R¹⁸ may together form methylene; wherein R¹⁴ and R¹⁸ may be independently optionally substituted on carbon by one or more R²⁴; and wherein if said heterocyclyl contains an —NH— moiety that nitrogen may be optionally substituted by a group selected from R²⁵;

R¹⁰, R¹⁵, R¹⁹, R²¹ and R²⁵ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁶, R¹⁷, R²² and R²³ are independently selected from a direct bond, —O—, —N(R²⁶)—, —C(O)—, —N(R²⁷)C(O)—, —C(O)N(R²⁸)—, —S(O)ₚ—, —SO₂N(R²⁹)— or —N(R³⁰)SO₂—; wherein R²⁶, R²⁷,

R²⁸, R²⁹ and R³⁰ are independently selected from hydrogen or C₁₋₄alkyl and p is 0-2;

R²⁰ and R²⁴ are independently selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;
with the proviso that said compound is not:
cis(±)-methyl 2-(3-azido-4-{[(3,4-dichloro-5-methyl-1H-pyrrol-2-yl)carbonyl]amino}piperidin-1-yl)-1,3-thiazole-5-carboxylate; or
cis(±)-2-(3-azido-4-{[(3,4-dichloro-5-methyl-1H-pyrrol-2-yl)carbonyl]amino}piperidin-1-yl)-1,3-thiazole-5-carboxylic acid.

The invention also provides a compound which is one of the Examples described herein.

The invention also provides a compound which is one of the Examples or a pharmaceutically acceptable salt thereof.

The invention also provides a compound which is one of Examples 12, 14, 19, 20, 25, 29, 52, 53, 72, 108 or 125 a pharmaceutically acceptable salt thereof.

### DETAILED DESCRIPTION OF THE INVENTION

### Definitions

In this specification the term alkyl includes both straight and branched chain alkyl groups. For example, “C₁₋₄alkyl” includes methyl, ethyl, propyl, isopropyl and t-butyl. However references to individual alkyl groups such as propyl are specific for the straight chain version only. An analogous convention applies to other generic terms.

Where optional substituents are chosen from one or more groups it is to be understood that this definition includes all substituents being chosen from one of the specified groups or the substituents being chosen from two or more of the specified groups.

R⁴ and R⁵ together with the carbon to which they are attached may form a 3-6 membered carbocyclic or heterocyclic ring. Said “3-6 membered carbocyclic or heterocyclic ring” is therefore fused to the piperidino ring of formula (I) in a spiro manner. A “carbocyclic ring” is a saturated, partially saturated or unsaturated, monocyclic carbon ring that contains 3-6 atoms, one of which is shared with the piperidine of formula (I); wherein a —CH₂— group can optionally be replaced by a —C(O)—. A “heterocyclic ring” is a saturated, partially saturated or unsaturated, monocyclic ring containing 3-6 atoms, one of which is shared with the piperidine of formula (I); of which at least one atom is chosen from nitrogen, sulphur or oxygen, wherein a —CH₂— group can optionally be replaced by a —C(O)— and a ring sulphur atom may be optionally oxidised to form the S-oxide(s). Suitable examples of a “heterocyclic ring” would be 1,3-dioxolan-2-yl and 1,3-dioxanyl. Said ring “may be optionally spiro-fused to a further 3-6 membered carbocyclic or heterocyclic ring”. In this instance this further 3-6 membered carbocyclic or heterocyclic ring would share an atom in common with the original ring in a spiro manner. An example of this would be 5,7-dioxaspiro[2.5]octyl.

R¹¹ and R¹² together with the nitrogen to which they are attached form a heterocyclic group. A “heterocyclic group” is a saturated, partially saturated or unsaturated, mono or bicyclic ring containing 4-12 atoms of which at least one atom is nitrogen and the others are chosen from carbon, nitrogen, sulphur or oxygen, which may, unless otherwise specified, be carbon or nitrogen linked, wherein a —CH₂— group can optionally be replaced by a —C(O)— and a ring nitrogen and/or a ring sulphur atom may be optionally oxidised to form the N- or S-oxide(s).

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring nitrogen and/or a ring sulphur atom may be optionally oxidised to form the N- or S-oxide(s). In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides. In a further aspect of the invention a “heterocyclyl” is an unsaturated, carbon-linked, monocyclic ring containing 5 or 6 atoms of which at least one atom is chosen from nitrogen, sulphur or oxygen. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. Further examples and suitable values of the term “heterocyclyl” are thiazolyl, quinolinyl, benzothiazolyl, pyrimidinyl and pyridinyl.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl.

An example of “C₁₋₄alkanoyloxy” is acetoxy.

Examples of “C₁₋₄alkoxycarbonyl” include methoxycarbonyl, ethoxycarbonyl, n- and t-butoxycarbonyl.

Examples of “C₁₋₄alkoxycarbonylamino” include methoxycarbonylamino, ethoxycarbonylamino, n- and t-butoxycarbonylamino.

Examples of “C₁₋₄alkoxy” include methoxy, ethoxy and propoxy.

Examples of “C₁₋₄alkanoylamino” include formamido, acetamido and propionylamino.

Examples of “C₁₋₄alkylS(O)ₐ wherein a is 0 to 2” include methylthio, ethylthio, methylsulphinyl, ethylsulphinyl, mesyl and ethylsulphonyl.

Examples of “C₁₋₄alkanoyl” include formyl, propionyl and acetyl.

Examples of “N—(C₁₋₄alkyl)amino” include methylamino and ethylamino.

Examples of “N,N—(C₁₋₄alkyl)₂amino” include di-N-methylamino, di-(N-ethyl)amino and N-ethyl-N-methylamino.

Examples of “C₂₋₄alkenyl” are vinyl, allyl and 1-propenyl. Examples of “C₂₋₄alkynyl” are ethynyl, 1-propynyl and 2-propynyl.

Examples of “N—(C₁₋₄alkyl)sulphamoyl” are N-(methyl)sulphamoyl and N-(ethyl)sulphamoyl.

Examples of “N,N—(C₁₋₄alkyl)₂sulphamoyl” are N,N-(dimethyl)sulphamoyl and N-(methyl)-N-(ethyl)sulphamoyl.

Examples of “N—(C₁₋₄alkyl)carbamoyl” are methylaminocarbonyl and ethylaminocarbonyl.

Examples of “N,N—(C₁₋₄alkyl)₂carbamoyl” are dimethylaminocarbonyl and methylethylaminocarbonyl.

Examples of “N—(C₁₋₄alkoxy)carbamoyl” are methoxyaminocarbonyl and isopropoxyaminocarbonyl.

Examples of “N—(C₁₋₄alkyl)-N—(C₁₋₄alkoxy)carbamoyl” are N-methyl-N-methoxyaminocarbonyl and N-methyl-N-ethoxyaminocarbonyl.

Examples of “C₃₋₆cycloalkyl” are cyclopropyl, cyclobutyl, cyclopropyl and cyclohexyl.

Examples of “N′—(C₁₋₄alkyl)ureido” are N′-methylureido and N′-isopropylureido. Examples of “N′,N′—(C₁₋₄alkyl)₂ureido” are N′N′-dimethylureido and N′-methyl-N′-isopropylureido.

Examples of “N′—(C₁₋₄alkyl)hydrazinocarbonyl” are N′-methylhydrazinocarbonyl and N′-isopropylhydrazinocarbonyl.

Examples of “N′,N′—(C₁₋₄alkyl)₂hydrazinocarbonyl” are N′N′-dimethylhydrazinocarbonyl and N′-methyl-N′-isopropylhydrazinocarbonyl.

Examples of “C₁₋₄alkylsulphonylamino” include methylsulphonylamino, isopropylsulphonylamino and t-butylsulphonylamino.

Examples of “C₁₋₄alkylsulphonylaminocarbonyl” include methylsulphonylaminocarbonyl, isopropylsulphonylaminocarbonyl and t-butylsulphonylaminocarbonyl.

Examples of “C₁₋₄alkylsulphonyl” include methylsulphonyl, isopropylsulphonyl and t-butylsulphonyl.

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium or potassium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, tromethamine, N-methyl d-glucamine and amino acids such as glycine or lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

Within the present invention it is to be understood that a compound of the formula (I) or a salt thereof may exhibit the phenomenon of tautomerism and that the formulae drawings within this specification can represent only one of the possible tautomeric forms. It is to be understood that the invention encompasses any tautomeric form which inhibits DNA gyrase and/or topoisomerase IV and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names. It will be appreciated by those skilled in the art that certain compounds of formula (I) contain an asymmetrically substituted carbon and/or sulphur atom, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which form possesses properties useful in the inhibition of DNA gyrase and/or topoisomerase IV, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DNA gyrase and/or topoisomerase IV by the standard tests described hereinafter.

It is also to be understood that certain compounds of the formula (I) and salts thereof can exist in solvated as well as unsolvated forms such as, for example, hydrated forms. It is to be understood that the invention encompasses all such solvated forms which inhibit DNA gyrase and/or topoisomerase IV.

Particular values of variable groups are as follows. Such values may be used where appropriate with any of the definitions, claims or embodiments defined hereinbefore or hereinafter. Each stated species represents a particular and independent aspect of the invention.

R¹ is C₁₋₄alkyl.

R¹ is methyl.

R² is halo.

R² is chloro.

R³ is halo.

R³ is chloro.

R¹ is methyl, R² is chloro and R³ is chloro.
W is —N(R⁷)—; where R⁷ is hydrogen.

R¹ is methyl, R² is chloro, R³ is chloro and W is NH.

Ring A is carbocyclyl.

Ring A is heterocyclyl; wherein if said heterocyclyl contains an —NH— moiety that nitrogen may be optionally substituted by a group selected from R¹⁰.

Ring A is heterocyclyl.

Ring A is thiazolyl.

Ring A is thiazolyl or pyridyl.

Ring A is thiazolyl, benzothiazolyl or pyridyl.

Ring A is thiazol-2-yl or pyrid-2-yl.

Ring A is thiazol-2-yl, benzothiazol-2-yl or pyrid-2-yl.

Ring A is thiazol-2-yl.

R¹ is methyl, R² is chloro, R³ is chloro, W is NH and Ring A is thiazolyl, benzothiazolyl or pyridyl.

R¹ is methyl, R² is chloro, R³ is chloro, W is NH and Ring A is thiazolyl.

R¹ is methyl, R² is chloro, R³ is chloro, W is NH and Ring A is thiazol-2-yl.

One of R⁴ and R⁵ is hydrogen and the other one is selected from azido or heterocyclyl; wherein R⁴ and R⁵ may be optionally substituted on carbon by one or more R¹⁴; and wherein if said heterocyclyl contains an —NH— moiety that nitrogen may be optionally substituted by a group selected from R¹⁵.

R⁴ and R⁵ are independently selected from an C₁₋₆alkyl or an C₁₋₆alkoxy group; wherein R⁴ and R⁵ may be optionally substituted on carbon by one or more R¹⁴.

R⁴ and R⁵ together form oxo, R¹¹R¹²N—N═ or R¹³O—N═.

R⁴ and R⁵ together with the carbon to which they are attached form 3-6 membered carbocyclic or heterocyclic ring; wherein R⁴ and R⁵ may be optionally substituted on carbon by one or more R¹⁴; and wherein if said heterocyclic ring contains an —NH— moiety that nitrogen may be optionally substituted by a group selected from R¹⁵.

R⁴ and R⁵ are selected from the following groups: (i) one of R⁴ and R⁵ is hydrogen and the other one is selected from azido, amino or heterocyclyl; or (ii) R⁴ and R⁵ are independently selected from a C₁₋₆alkoxy group; or (iii) R⁴ and R⁵ together form R¹³O—N═; or (iv) R⁴ and R⁵ together with the carbon to which they are attached form a 3-6 membered heterocyclic ring wherein said ring may be optionally spiro-fused to a further 3-6 membered carbocyclic ring; wherein R⁴ and R⁵ in any of groups (i)-(iv) may be optionally substituted on carbon by one or more R¹⁴; wherein

R¹³ is C₁₋₄alkyl;

R¹⁴ is selected from halo, cyano, C₁ alkyl or C₁₋₄alkoxy; or two R¹⁴ may together form methylene; wherein R¹⁴ and R¹⁸ may be independently optionally substituted on carbon by one or more R²⁴;

R²⁴ is selected from halo, cyano, hydroxy and methoxy.

R⁴ and R⁵ are selected from the following groups: (ii) R⁴ and R⁵ are independently selected from C₁₋₆alkoxy group; or (iii) R⁴ and R⁵ together form R¹³O—N═; or (iv) R⁴ and R⁵ together with the carbon to which they are attached form 3-6 membered heterocyclic ring; wherein R¹³ is C₁₋₄alkyl.

R⁴ and R⁵ are selected from the following groups: (i) one of R⁴ and R⁵ is hydrogen and the other one is selected from azido, amino, imidazolyl, 1,2,3-triazolyl or 1,2,4-triazolyl; or (ii) R⁴ and R⁵ are both methoxy; or (iii) R⁴ and R⁵ together form R¹³O—N═; or (iv) R⁴ and R⁵ together with the carbon to which they are attached form 1,3-dioxolanyl or 1,3-dioxanyl wherein said ring may be optionally spiro-fused to a further cyclopropyl ring; wherein R⁴ and R⁵ in any of groups (i)-(iv) may be optionally substituted on carbon by one or more R¹⁴; wherein

R¹³ is methyl;

R¹⁴ is selected from chloro, bromo, cyano, methyl, methoxy or ethoxy; or two R¹⁴ may together form methylene; wherein R¹⁴ and R¹⁸ may be independently optionally substituted on carbon by one or more R²⁴;

R²⁴ is selected from fluoro, cyano, hydroxy and methoxy.

R⁴ and R⁵ are selected from the following groups: (ii) R⁴ and R⁵ are both methoxy group; or (iii) R⁴ and R⁵ together form methoxyimino; or (iv) R⁴ and R⁵ together with the carbon to which they are attached form 1,3-dioxolanyl or 1,3-dioxanyl.

R⁴ and R⁵ are selected from the following groups: (i) one of R⁴ and R⁵ is hydrogen and the other one is selected from azido, amino, imidazol-1-yl, 1,2,3-triazol-1-yl, 4-methyl-1,2,3-triazol-1-yl, 4-cyano-1,2,3-triazol-1-yl, 4-hydroxymethyl-1,2,3-triazol-1-yl, 4-cyanomethyl-1,2,3-triazol-1-yl, 4-fluoromethyl-1,2,3-triazol-1-yl, 4-methoxymethyl-1,2,3-triazol-1-yl, 4-chloro-1,2,3-triazol-1-yl, 3-chloro-1,2,4-triazol-1-yl, 4-bromo-1,2,3-triazol-1-yl or 1,2,4-triazol-1-yl; or (ii) R⁴ and R⁵ are both methoxy; or (iii) R⁴ and R⁵ together form MeO—N═; or (iv) R⁴ and R⁵ together with the carbon to which they are attached form 1,3-dioxolanyl, 5-methoxy-1,3-dioxanyl, 5-ethoxy-1,3-dioxanyl, 5-hydroxymethyl-1,3-dioxanyl, 5,5-dimethyl-1,3-dioxanyl, 5,7-dioxaspiro[2.5]octyl, 5-methylene-1,3-dioxanyl or 1,3-dioxanyl.

R⁶ is carboxy, C₁₋₄alkanoyl, N—(C₁₋₄alkyl)carbamoyl, C₁₋₄alkoxycarbonyl or C₁₋₄alkenyloxycarbonyl; wherein R⁶ may be optionally substituted on carbon by one or more R¹⁸; wherein R¹⁸ is C₁₋₄alkoxy.

R⁶ is a substituent on carbon and is selected from carboxy, carbamoyl, C₁₋₄alkanoyl, N—(C₁₋₄alkyl)carbamoyl, N—(C₁₋₄alkoxy)carbamoyl, C₁₋₄alkoxycarbonyl, C₁₋₄alkenyloxycarbonyl or carbocyclyl-R¹⁶—; wherein R⁶ may be optionally substituted on carbon by one or more R¹⁸;

R¹⁶ is —N(R²⁷)C(O)—; wherein R²⁷ is hydrogen; and

R¹⁸ is C₁₋₄alkoxy.

R⁶ is a substituent on carbon and is selected from carboxy, carbamoyl, C₁₋₄alkanoyl, N—(C₁₋₄alkyl)carbamoyl, N—(C₁₋₄alkoxy)carbamoyl, C₁₋₄alkoxycarbonyl, C₁₋₄alkenyloxycarbonyl, carbocyclyl-R¹⁶— or heterocyclyl-R¹⁷—; wherein R⁶ may be optionally substituted on carbon by one or more R¹⁸; and wherein if said heterocyclyl contains an —NH— moiety that nitrogen may be optionally substituted by a group selected from R¹⁹;

R¹⁶ and R¹⁷ are independently selected from a direct bond and —N(R²⁷)C(O)—; wherein R²⁷ is hydrogen;

R¹⁸ is C₁₋₄alkoxy;

R¹⁹ is selected from C₁₋₄alkyl; wherein R¹⁹ may be independently optionally substituted on carbon by one or more R³¹; and

R³¹ is methoxy.

R⁶ is carboxy, formyl, acetyl, N-(methyl)carbamoyl, N-(ethyl)carbamoyl, methoxycarbonyl, ethoxycarbonyl, isopropoxycarbonyl or allyloxycarbonyl; wherein R⁶ may be optionally substituted on carbon by one or more R¹⁸; wherein R¹⁸ is methoxy.

R⁶ is a substituent on carbon and is selected from carboxy, carbamoyl, formyl, acetyl, N-(methyl)carbamoyl, N-(ethyl)carbamoyl, N-(prop-2-yl)carbamoyl, N-(2-methylprop-2-yl)carbamoyl, N-(methoxy)carbamoyl, methoxycarbonyl, ethoxycarbonyl, isopropoxycarbonyl, 1-propen-3-yloxycarbonyl or cyclopropyl-R¹⁶—; wherein R⁶ may be optionally substituted on carbon by one or more R¹⁸;

R¹⁶ is —N(R²⁷)C(O)—; wherein R²⁷ is hydrogen; and

R¹⁸ is methoxy.

R⁶ is a substituent on carbon and is selected from carboxy, carbamoyl, formyl, acetyl, N-(methyl)carbamoyl, N-(ethyl)carbamoyl, N-(prop-2-yl)carbamoyl, N-(2-methylprop-2-yl)carbamoyl, N-(methoxy)carbamoyl, methoxycarbonyl, ethoxycarbonyl, isopropoxycarbonyl, 1-propen-3-yloxycarbonyl, cyclopropyl-R¹⁶—, 1H-1,2,4-triazol-5-yl-R¹⁷— or imidazol-2-yl-R¹⁷—; wherein R⁶ may be optionally substituted on carbon by one or more R¹⁸; wherein said imidazolyl or triazolyl may be optionally substituted on nitrogen by a group selected from R¹⁹;

R¹⁶ and R¹⁷ are independently selected from a direct bond and —N(R²⁷)C(O)—; wherein

R²⁷ is hydrogen;

R¹⁸ is methoxy;

R¹⁹ is selected from methyl; wherein R¹⁹ may be independently optionally substituted on carbon by one or more R³¹; and

R³¹ is methoxy.

R⁶ is carboxy, formyl, acetyl, N-(methyl)carbamoyl, N-(2-methoxyethyl)carbamoyl, methoxycarbonyl, ethoxycarbonyl, isopropoxycarbonyl or allyloxycarbonyl.

R⁶ is a substituent on carbon and is selected from carboxy, carbamoyl, formyl, acetyl, methoxycarbonyl, ethoxycarbonyl, isopropoxycarbonyl, N-(methoxy)carbamoyl, N-(2-methoxyethyl)carbamoyl, N-(1,3-dimethoxyprop-2-yl)carbamoyl, N-(cyclopropyl)carbamoyl, N-(1-methoxyprop-2-yl)carbamoyl, N-(1,3-dimethoxy-2-methoxymethylprop-2-yl)carbamoyl, 1-propen-3-yloxycarbonyl or N-(methyl)carbamoyl.

R⁶ is a substituent on carbon and is selected from carboxy, carbamoyl, formyl, acetyl, methoxycarbonyl, ethoxycarbonyl, isopropoxycarbonyl, N-(methoxy)carbamoyl, N-(2-methoxyethyl)carbamoyl, N-(1,3-dimethoxyprop-2-yl)carbamoyl, N-(cyclopropyl)carbamoyl, N-(1-methoxyprop-2-yl)carbamoyl, N-(1,3-dimethoxy-2-methoxymethylprop-2-yl)carbamoyl, 1-propen-3-yloxycarbonyl, N-(methyl)carbamoyl, 1-methoxymethylimidazol-2-yl, imidazol-2-yl or 1H-1-methyl-1,2,4-triazol-5-yl.

m is 1 or 2; wherein the values of R⁶ may be the same or different.

m is 1.

m is 2; wherein the values of R⁶ may be the same or different.

Therefore, in a further aspect of the invention there is provided a compound of formula (I) (as depicted above) wherein:

R¹ is C₁₋₄alkyl;

R² is halo;

R³ is halo;

W is —N(R⁷)—; where R⁷ is hydrogen;

Ring A is heterocyclyl;

R⁴ and R⁵ are selected from the following groups: (ii) R⁴ and R⁵ are independently selected from C₁₋₆alkoxy group; or (iii) R⁴ and R⁵ together form R¹³O—N═; or (iv) R⁴ and R⁵ together with the carbon to which they are attached form 3-6 membered heterocyclic ring; wherein R¹³ is C₁₋₄alkyl;

R⁶ is carboxy, C₁₋₄alkanoyl, N—(C₁₋₄alkyl)carbamoyl, C₁₋₄alkoxycarbonyl or C₁₋₄alkenyloxycarbonyl; wherein R⁶ may be optionally substituted on carbon by one or more R¹⁸; wherein R¹⁸ is C₁₋₄alkoxy;

m is 1 or 2; wherein the values of R⁶ may be the same or different;

or a pharmaceutically acceptable salt thereof.

Therefore, in a further aspect of the invention there is provided a compound of formula (I) (as depicted above) wherein:

R¹ is C₁₋₄alkyl;

R² is halo;

R³ is halo;

W is —N(R⁷)—; where R⁷ is hydrogen;

Ring A is thiazolyl, benzothiazolyl or pyridyl;

R⁴ and R⁵ are selected from the following groups: (i) one of R⁴ and R⁵ is hydrogen and the other one is selected from azido, amino or heterocyclyl; or (ii) R⁴ and R⁵ are independently selected from a C₁₋₆alkoxy group; or (iii) R⁴ and R⁵ together form R¹³O—N═; or (iv) R⁴ and R⁵ together with the carbon to which they are attached form a 3-6 membered heterocyclic ring wherein said ring may be optionally spiro-fused to a further 3-6 membered carbocyclic ring; wherein R⁴ and R⁵ in any of groups (i)-(iv) may be optionally substituted on carbon by one or more R¹⁴;

R⁶ is a substituent on carbon and is selected from carboxy, carbamoyl, N—(C₁₋₄alkyl)carbamoyl, N—(C₁₋₄alkoxy)carbamoyl, C₁₋₄alkoxycarbonyl, C₁₋₄alkenyloxycarbonyl, carbocyclyl-R¹⁶— or heterocyclyl-R¹⁷—; wherein R⁶ may be optionally substituted on carbon by one or more R¹⁸; and wherein if said heterocyclyl contains an —NH— moiety that nitrogen may be optionally substituted by a group selected from R¹⁹;

m is 1 or 2; wherein the values of R⁶ may be the same or different;

R¹³ is C₁₋₄alkyl;

R¹⁴ is selected from halo, cyano, C₁₋₄alkyl or C₁₋₄alkoxy; or two R¹⁴ may together form methylene; wherein R¹⁴ and R¹⁸ may be independently optionally substituted on carbon by one or more R²⁴;

R¹⁶ and R¹⁷ are independently selected from a direct bond and —N(R²⁷)C(O)—; wherein R²⁷ is hydrogen;

R¹⁸ is C₁₋₄alkoxy;

R¹⁹ is selected from C₁₋₄alkyl; wherein R¹⁹ may be independently optionally substituted on carbon by one or more R³¹;

R²⁴ is selected from halo, cyano, hydroxy and methoxy; and

R³¹ is methoxy;

or a pharmaceutically acceptable salt thereof.

Therefore, in a further aspect of the invention there is provided a compound of formula (I) (as depicted above) wherein:

R¹ is C₁₋₄alkyl;

R² is halo;

R³ is halo;

W is —N(R⁷)—; where R⁷ is hydrogen;

Ring A is thiazolyl, benzothiazolyl or pyridyl;

R⁴ and R⁵ are selected from the following groups: (i) one of R⁴ and R⁵ is hydrogen and the other one is selected from azido, amino or heterocyclyl; or (ii) R⁴ and R⁵ are independently selected from a C₁₋₆alkoxy group; or (iii) R⁴ and R⁵ together form R¹³O—N═; or (iv) R⁴ and R⁵ together with the carbon to which they are attached form a 3-6 membered heterocyclic ring wherein said ring may be optionally spiro-fused to a further 3-6 membered carbocyclic ring; wherein R⁴ and R⁵ in any of groups (i)-(iv) may be optionally substituted on carbon by one or more R¹⁴;

R⁶ is a substituent on carbon and is selected from carboxy, carbamoyl, C₁₋₄alkanoyl, N—(C₁₋₄alkyl)carbamoyl, N—(C₁₋₄alkoxy)carbamoyl, C₁₋₄alkoxycarbonyl, C₁₋₄alkenyloxycarbonyl or carbocyclyl-R¹⁶—; wherein R⁶ may be optionally substituted on carbon by one or more R¹⁸;

m is 1 or 2; wherein the values of R⁶ may be the same or different;

R¹³ is C₁₋₄alkyl;

R¹⁴ is selected from halo, cyano, C₁₋₄alkyl or C₁₋₄alkoxy; or two R¹⁴ may together form methylene; wherein R¹⁴ and R¹⁸ may be independently optionally substituted on carbon by one or more R²⁴;

R¹⁶ is N(R²⁷)C(O)—; wherein R²⁷ is hydrogen;

R¹⁸ is C₁₋₄alkoxy;

R²⁴ is selected from halo, cyano, hydroxy and methoxy;

or a pharmaceutically acceptable salt thereof.

Therefore, in a further aspect of the invention there is provided a compound of formula (I) (as depicted above) wherein:

R¹ is methyl;

R² is chloro;

R³ is chloro;

W is —N(R⁷)—; where R⁷ is hydrogen;

Ring A is thiazol-2-yl or pyrid-2-yl;

R⁴ and R⁵ are selected from the following groups: (ii) R⁴ and R⁵ are both methoxy group; or (iii) R⁴ and R⁵ together form methoxyimino; or (iv) R⁴ and R⁵ together with the carbon to which they are attached form 1,3-dioxolanyl or 1,3-dioxanyl;

R⁶ is carboxy, formyl, acetyl, N-(methyl)carbamoyl, N-(2-methoxyethyl)carbamoyl, methoxycarbonyl, ethoxycarbonyl, isopropoxycarbonyl or allyloxycarbonyl;

m is 1 or 2; wherein the values of R⁶ may be the same or different;

or a pharmaceutically acceptable salt thereof.

Therefore, in a further aspect of the invention there is provided a compound of formula (I) (as depicted above) wherein:

R¹ is methyl;

R² is chloro;

R³ is chloro;

W is —N(R⁷)—; where R⁷ is hydrogen;

Ring A is thiazol-2-yl, benzothiazol-2-yl or pyrid-2-yl;

R⁴ and R⁵ are selected from the following groups: (i) one of R⁴ and R⁵ is hydrogen and the other one is selected from azido, amino, imidazol-1-yl, 1,2,3-triazol-1-yl, 4-methyl-1,2,3-triazol-1-yl, 4-cyano-1,2,3-triazol-1-yl, 4-hydroxymethyl-1,2,3-triazol-1-yl, 4-cyanomethyl-1,2,3-triazol-1-yl, 4-fluoromethyl-1,2,3-triazol-1-yl, 4-methoxymethyl-1,2,3-triazol-1-yl, 4-chloro-1,2,3-triazol-1-yl, 3-chloro-1,2,4-triazol-1-yl, 4-bromo-1,2,3-triazol-1-yl or 1,2,4-triazol-1-yl; or (ii) R⁴ and R⁵ are both methoxy; or (iii) R⁴ and R⁵ together form MeO—N═; or (iv) R⁴ and R⁵ together with the carbon to which they are attached form 1,3-dioxolanyl, 5-methoxy-1,3-dioxanyl, 5-ethoxy-1,3-dioxanyl, 5-hydroxymethyl-1,3-dioxanyl, 5,5-dimethyl-1,3-dioxanyl, 5,7-dioxaspiro[2.5]octyl, 5-methylene-1,3-dioxanyl or 1,3-dioxanyl;

R⁶ is a substituent on carbon and is selected from carboxy, carbamoyl, formyl, acetyl, methoxycarbonyl, ethoxycarbonyl, isopropoxycarbonyl, N-(methoxy)carbamoyl, N-(2-methoxyethyl)carbamoyl, N-(1,3-dimethoxyprop-2-yl)carbamoyl, N-(cyclopropyl)carbamoyl, N-(1-methoxyprop-2-yl)carbamoyl, N-(1,3-dimethoxy-2-methoxymethylprop-2-yl)carbamoyl, 1-propen-3-yloxycarbonyl, N-(methyl)carbamoyl, 1-methoxymethylimidazol-2-yl, imidazol-2-yl or 1H-1-methyl-1,2,4-triazol-5-yl.

m is 1 or 2; wherein the values of R⁶ may be the same or different;

or a pharmaceutically acceptable salt thereof.

Therefore, in a further aspect of the invention there is provided a compound of formula (I) (as depicted above) wherein:

R¹ is methyl;

R² is chloro;

R³ is chloro;

W is —N(R⁷)—; where R⁷ is hydrogen;

Ring A is thiazol-2-yl, benzothiazol-2-yl or pyrid-2-yl;

R⁴ and R⁵ are selected from the following groups: (i) one of R⁴ and R⁵ is hydrogen and the other one is selected from azido, amino, imidazol-1-yl, 1,2,3-triazol-1-yl, 4-methyl-1,2,3-triazol-1-yl, 4-cyano-1,2,3-triazol-1-yl, 4-hydroxymethyl-1,2,3-triazol-1-yl, 4-cyanomethyl-1,2,3-triazol-1-yl, 4-fluoromethyl-1,2,3-triazol-1-yl, 4-methoxymethyl-1,2,3-triazol-1-yl, 4-chloro-1,2,3-triazol-1-yl, 3-chloro-1,2,4-triazol-1-yl, 4-bromo-1,2,3-triazol-1-yl or 1,2,4-triazol-1-yl; or (ii) R⁴ and R⁵ are both methoxy; or (iii) R⁴ and R⁵ together form MeO—N═; or (iv) R⁴ and R⁵ together with the carbon to which they are attached form 1,3-dioxolanyl, 5-methoxy-1,3-dioxanyl, 5-ethoxy-1,3-dioxanyl, 5-hydroxymethyl-1,3-dioxanyl, 5,5-dimethyl-1,3-dioxanyl, 5,7-dioxaspiro[2.5]octyl, 5-methylene-1,3-dioxanyl or 1,3-dioxanyl;

R⁶ is a substituent on carbon and is selected from carboxy, carbamoyl, formyl, acetyl, methoxycarbonyl, ethoxycarbonyl, isopropoxycarbonyl, N-(methoxy)carbamoyl, N-(2-methoxyethyl)carbamoyl, N-(1,3-dimethoxyprop-2-yl)carbamoyl, N-(cyclopropyl)carbamoyl, N-(1-methoxyprop-2-yl)carbamoyl, N-(1,3-dimethoxy-2-methoxymethylprop-2-yl)carbamoyl, 1-propen-3-yloxycarbonyl or N-(methyl)carbamoyl;

m is 1 or 2; wherein the values of R⁶ may be the same or different;

or a pharmaceutically acceptable salt thereof.

### Preparation of Invention Compounds

In a further aspect the present invention provides a process for preparing a compound of formula (I) or a pharmaceutically-acceptable salt thereof.

Thus, the present invention also provides that the compounds of the formula (I) and pharmaceutically-acceptable salts thereof, can be prepared by a process as follows (wherein the variables are as defined above unless otherwise stated):

Process a) for compounds of formula (I) wherein W is —C(R⁸)(R⁹)—; converting a compound of formula (II):

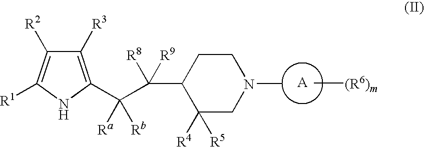

wherein R^{a} is cyano and R^{b} is dimethyamino or diethylamino; or R^{a} and R^{b} are independently selected from C₁₋₄alkylthio; or R^{a} and R^{b} together form 1,3-dithianyl or 1,3-dithiolanyl; into a compound of formula (I); or
Process b) for compounds of formula (I) wherein W is —O—; reacting a compound of formula (III):

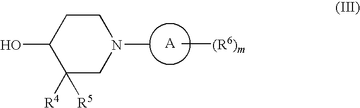

with a compound of formula (IV):

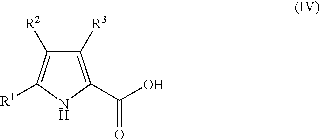

or
Process c) for compounds of formula (I) wherein W is —N(R⁷)—; reacting a compound of formula (V):

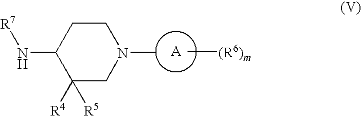

with a compound of formula (IV) or an activated acid derivative thereof; or
Process d) for compounds of formula (I) wherein W is —C(R⁸)(R⁹)—; reacting a compound of formula (VI):

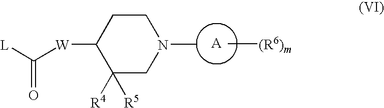

wherein L is a displaceable group; with a compound of formula (VII):

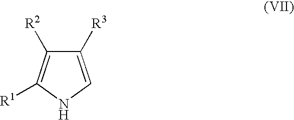

or
Process e) for compounds of formula (I) wherein W is —C(R⁸)(R⁹)—; reacting a compound of formula (VIII):

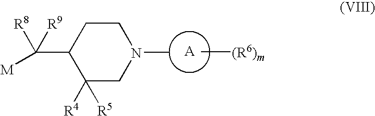

wherein M is an organometallic group; with a compound of formula (IX):

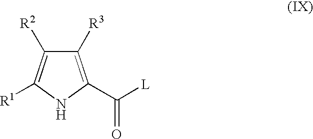

wherein L is a displaceable group; or
Process f) reacting a compound of formula (X):

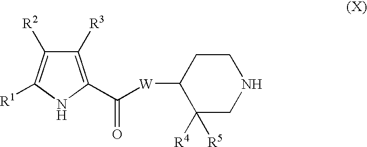

with a compound of formula (XI):

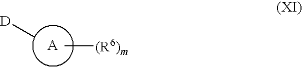

wherein D is a displaceable group; or
Process g) for compounds of formulas (I) wherein R⁴ and R⁵ together form R¹¹R¹²N—N═ or R¹³O—N═, by reacting a compound of formula (I) wherein R⁴ and R⁵ together form oxo with an amine of formula R¹¹R¹²N—NH₂ or R¹³O—NH²; or
Process h) for compounds of formulas (I) wherein R⁴ and R⁵ together with the carbon to which they are attached form a heterocyclic ring selected from 1,3-dioxolan-2-yl or 1,3-dioxan-2-yl, by reacting a compound of formula (I) wherein R⁴ and R⁵ together form oxo with 1,2-dihydroxyethane or 1,3-dihydroxypropane; and thereafter if necessary or desirable:
i) converting a compound of the formula (I) into another compound of the formula (I);
ii) removing any protecting groups;
iii) forming a pharmaceutically acceptable salt.

L is a displaceable group. Suitable values for L include halo, for example chloro and bromo, pentafluorophenoxy and 2,5-oxopyrrolidin-1-yloxy.

D is a displaceable group. Suitable values for D include halo, for example chloro, bromo and iodo, tosylate and mesylate.

M is an organometallic group, suitable values for M include organocuprates, for example CuLi, organozincs, Zn, or a Grignard reagent for example MgG where G is halo for example chloro.

Specific reaction conditions for the above reaction are as follows.

Process a) Compounds of formula (II) may be converted into compounds of formula (I):
(i) where R^{a} is cyano and R^{b} is dimethyamino or diethylamino; in the presence of a base for example sodium hydroxide, in a suitable solvent for example aqueous methanol at room temperature.
(ii) wherein or R^{a} and R^{b} are independently selected from C₁₋₄alkylthio; or R^{a} and R^{b} together form 1,3-dithianyl or 1,3-dithiolanyl; in the presence of a reagent such as a mercury, copper or silver salt for example Hg(ClO₄)₂, CuCl₂ or AgNO₃/Ag₂O in the presence of a suitable solvent for example methanol, acetone or ethanol from a temperature ranging from room temperature to reflux.

Compounds of formula (II) may be prepared according to Scheme 1:

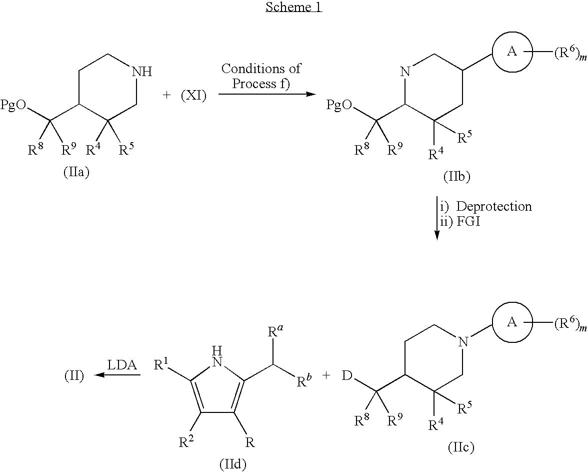

wherein Pg is a hydroxy protecting group as defined hereinbelow; and D is a displaceable group as defined hereinabove.

Deprotection of hydroxy protecting groups are well known in the art. Examples of such deprotections are given hereinbelow.

FGI stands for Functional Group Interconversion. In the above scheme such conversions between a hydroxy group and a D group are well known in the art and are well within the capabilities of a person skilled in the art.

Compounds of formula (IIa) and (IId) are known in the literature, or they are prepared by standard processes known in the art.

Process b) Compounds of formula (III) and (IV) may be reacted together may be reacted together in the presence of a coupling reagent, for example dicyclohexylcarbodiimide or EDC, in a suitable solvent, for example dichloromethane, THF or diethylether.

Compounds of formula (III) may be prepared according to Scheme 2:

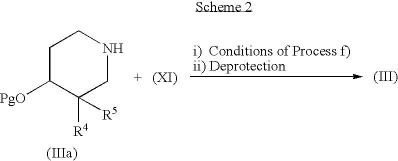

wherein Pg is a hydroxy protecting group as defined hereinbelow.

Deprotection of hydroxy protecting groups are well known in the art. Examples of such deprotections are given hereinbelow.

Compounds of formula (IIIa) and (IV) are commercially available compounds, or they are known in the literature, or they are prepared by standard processes known in the art.

Process c) Compounds of formula (V) and (IV) may be coupled together in the presence of a suitable coupling reagent. Standard peptide coupling reagents known in the art can be employed as suitable coupling reagents, or for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Compounds of formula (V) may be prepared according to Scheme 3:

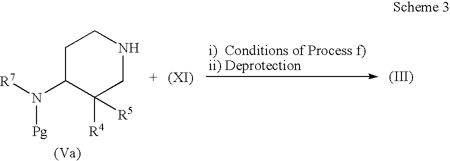

wherein Pg is a amino protecting group as defined hereinbelow. The skilled reader will appreciate that where R⁷ is hydrogen, this hydrogen also needs protecting by way of a suitable protecting group.

Deprotection of amino protecting groups are well known in the art. Examples of such deprotections are given hereinbelow.

Compounds of formula (Va) are commercially available compounds, or they are known in the literature, or they are prepared by standard processes known in the art.

Process d) Compounds of formula (VI) and (VII) may be reacted in a suitable solvent such a DCM or 1,2-dichloroethane, optionally in the presence of a Lewis acid, for example A1Cl₃, from 0° C. to room temperature.

Compounds of formula (VI) may be prepared according to Scheme 4:

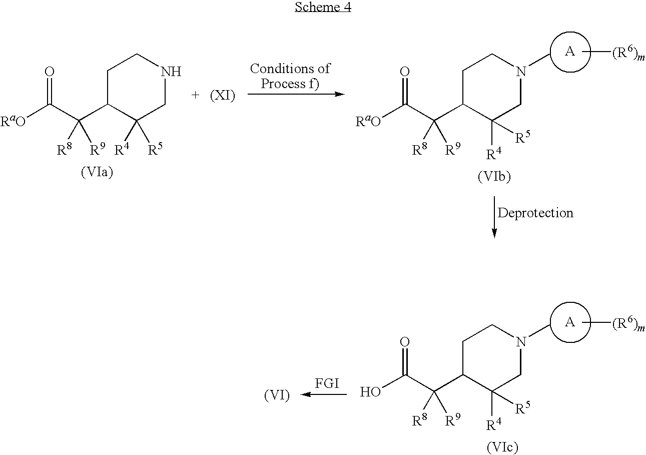

wherein R^{a}OC(O) is an ester group.

Suitable values for R^{a} include C₁₋₆alkyl. Deprotection of the R^{a} carboxy protecting group may be achieved under standard conditions, for example acid or base hydrolysis, such as those conditions give hereinbelow.

FGI stands for Functional Group Interconversion. In the above scheme such conversions between an acid group and a —C(O)L group are well known in the art and are well within the capabilities of a person skilled in the art.

Compounds of formula (VIa) and (VII) are commercially available compounds, or they are known in the literature, or they are prepared by standard processes known in the art.

Process e) Compounds of formula (VIII) and (IX) may be reacted in a suitable aprotic solvent such as THF or ether, at temperatures in the range of −78° C. to 0° C.

Compounds of formula (VIII) may be prepared from compounds of formula (IIc) under standard conditions known in the art. For example where M is an organocuprous reagent such compounds could be prepared according to Scheme 5:

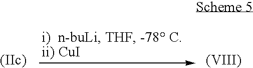

Compounds of formula (IX) are commercially available compounds, or they are known in the literature, or they are prepared by standard processes known in the art.

Process f) Compounds of formula (X) and (XI) may be reacted in a suitable solvent such as DMF, N-methylpyrrolidinone or dimethylacetamide in the presence of a base such as triethylamine or diisopropylethylamine under thermal conditions or a microwave reactor.

Compounds of formula (X) may be prepared according to Scheme 6:

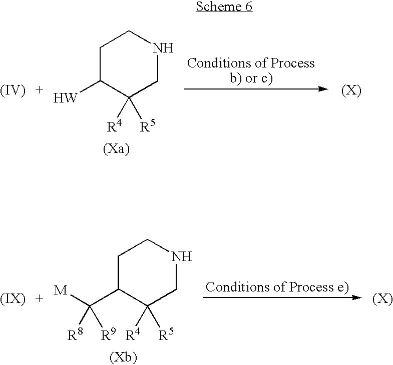

wherein M is an organometallic group as defined hereinabove.

Compounds of formula (Xa), (Xb) and (XI) are commercially available compounds, or they are known in the literature, or they are prepared by standard processes known in the art.

Process g) a compound of formula (I) wherein R⁴ and R⁵ together form oxo may be reacted with an amine of formula R¹¹R¹²N—NH₂ or R¹³O—NH₂ optionally in a protic solvent, for example methanol or ethanol, optionally with a weak base such as sodium acetate at a temperature of room temperature to reflux.

Compounds of formula R¹¹R¹²N—NH₂ or R¹³O—NH₂ are commercially available compounds, or they are known in the literature, or they are prepared by standard processes known in the art.

Process h) a compound of formula (I) wherein R⁴ and R⁵ together form oxo may be reacted with 1,2-dihydroxyethane or 1,3-dihydroxypropane in a hydrocarbon solvent, for example toluene, benzene or xylene under Dean-Starke conditions with a catalyst such as toluene sulphonic acid or methane sulphonic acid.

The formation of a pharmaceutically-acceptable salt is within the skill of an ordinary organic chemist using standard techniques.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. The reagents used to introduce such ring substituents are either commercially available or are made by processes known in the art.

Introduction of substituents into a ring may convert one compound of the formula (I) into another compound of the formula (I). Such reactions and modifications include, for example, introduction of a substituent by means of an aromatic substitution reaction, reduction of substituents, alkylation of substituents, oxidation of substituents, esterification of substituents, amidation of substituents, formation of heteroaryl rings. The reagents and reaction conditions for such procedures are well known in the chemical art. Particular examples of aromatic substitution reactions include the introduction of alkoxides, diazotization reactions followed by introduction of thiol group, alcohol group, halogen group. Examples of modifications include; oxidation of alkylthio to alkylsulphinyl or alkylsulphonyl.

The skilled organic chemist will be able to use and adapt the information contained and referenced within the above references, and accompanying Examples therein and also the Examples herein, to obtain necessary starting materials, and products. If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples. It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art, or they may be removed during a later reaction step or work-up.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

### Enzyme Potency Testing Methods

Compounds may be tested for inhibition of GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays can be performed in multiwell plates in 100 μl reactions containing: 50 mM HEPES buffer pH 7.5, 75 mM ammonium acetate, 5.5 mM magnesium chloride, 0.5 mM ethylenediaminetetraacetic acid, 5% glycerol, 1 mM 1,4-Dithio-DL-threitol, 200 nM bovine serum albumin, 5 μg/ml sheared salmon sperm DNA, 2.5 nM E. coli GyrA, 2.5 nM E. coli GyrB, 250 μM ATP, and compound in dimethylsulfoxide. Reactions can be quenched with 150 μl of ammonium molybdate/malachite green detection reagent containing 1.2 mM malachite green hydrochloride, 8.5 mM ammonium molybdate tetrahydrate, and 1 M hydrochloric acid. Plates can be read in an absorbance plate reader at 650 nm and percent inhibition values may be calculated using dimethylsulfoxide (2%)-containing reactions as 0% inhibition and novobiocin-containing (2 μM) reactions as 100% inhibition controls. Compound potency can be based on IC₅₀ measurements determined from reactions performed in the presence of 10 different compound concentrations.

Compounds may be tested for inhibition of topoisomerase IV ATPase activity as described above for GyrB except the 100 μl reactions may contain the following: 20 mM TRIS buffer pH 8, 50 mM ammonium acetate, 8 mM magnesium chloride, 5% glycerol, 5 mM 1,4-Dithio-DL-threitol, 0.005% Brij-35, 5 μg/ml sheared salmon sperm DNA, 2.5 nM E. coli ParC, 2.5 nM E. coli ParE, 160 μM ATP, and compound in dimethylsulfoxide. Compound potency may be based on IC₅₀ measurements determined from reactions performed in the presence of 10 different compound concentrations. Compounds of the Examples could be expected to have IC₅₀ values of <200 μg/ml in one or both assays described herein above. For Example the following results were obtained in a GyrB ATPase inhibition activity assay substantially similar to the above wherein the figure quoted may be an average of two or more results

| Ex | IC50 (μM) |
|---|---|
| 1 | 0.463 |
| 2 | 0.487 |
| 3 | 0.27 |
| 5 | 0.226 |
| 6 | 0.0791 |
| 7 | 0.0185 |
| 8 | 0.0736 |
| 9 | 0.0522 |
| 10 | 0.169 |
| 11 | 0.0178 |
| 12 | <0.0831 |
| 13 | <0.069 |
| 14 | <0.00977 |
| 16 | <0.00977 |
| 17 | <0.00977 |
| 18 | <0.00977 |
| 19 | <0.00977 |
| 20 | <0.0671 |
| 23 | <0.00977 |
| 24 | <0.00977 |
| 25 | <0.00977 |
| 26 | <0.00977 |
| 27 | <0.0635 |
| 28 | <0.00977 |
| 29 | <0.00977 |
| 30 | 0.0861 |
| 31 | 0.0494 |
| 32 | 0.0593 |
| 33 | 0.0369 |
| 34 | 0.0668 |
| 36 | 0.0755 |
| 38 | 0.0909 |
| 39 | 0.0334 |
| 40 | 0.0519 |
| 41 | 0.0534 |
| 42 | <0.0756 |
| 43 | <0.00977 |
| 44 | <0.00977 |
| 45 | <0.00977 |
| 46 | <0.00977 |
| 47 | 0.0257 |
| 48 | 0.0365 |
| 49 | <0.00977 |
| 50 | <0.00977 |
| 51 | <0.122 |
| 52 | <0.00977 |
| 53 | <0.00977 |
| 54 | <0.00977 |
| 55 | <0.00977 |
| 56 | <0.00977 |
| 57 | <0.00977 |
| 61 | <0.00977 |
| 64 | 0.155 |
| 65 | <0.00977 |
| 66 | <0.00977 |
| 68 | 0.291 |
| 70 | 0.0124 |
| 71 | 0.04 |
| 82 | <0.00977 |
| 92 | <0.00977 |
| 100 | <0.00977 |
| 101 | <0.00977 |
| 102 | <0.00977 |
| 103 | <0.00977 |
| 104 | <0.00977 |
| 105 | <0.00977 |
| 106 | <0.432 |
| 107 | <0.00977 |
| 108 | <0.00977 |
| 109 | <0.00977 |
| 110 | <0.00977 |
| 111 | <0.00977 |
| 112 | <0.00977 |
| 113 | <0.00977 |
| 114 | <0.00977 |
| 115 | <0.00977 |
| 116 | <0.00977 |
| 117 | <0.00977 |
| 118 | <0.00977 |
| 119 | 0.0123 |
| 120 | 0.0196 |
| 121 | 0.0369 |
| 122 | <0.00977 |
| 123 | 0.218 |
| 124 | <0.00977 |
| 125 | <0.00977 |
| 126 | 0.0327 |

### Bacterial Susceptibility Testing Methods

Compounds may be tested for antimicrobial activity by susceptibility testing in liquid media. Compounds may be dissolved in dimethylsulfoxide and tested in 10 doubling dilutions in the susceptibility assays. The organisms used in the assay may be grown overnight on suitable agar media and then suspended in a liquid medium appropriate for the growth of the organism. The can be was a 0.5 McFarland and a further 1 in 10 dilution can be made into the same liquid medium to prepare the final organism suspension in 100 pt. Plates can be incubated under appropriate conditions at 37° C. for 24 hrs prior to reading. The Minimum Inhibitory Concentration (MIC) may be determined as the lowest drug concentration able to reduce growth by 80% or more. Example 1 had an MIC of 1 μg/ml against Streptococcus pneumoniae. Other examples are provided in the following table.

| Example | MIC | MIC | MIC | MIC | |
|---|---|---|---|---|---|
| No. | HIN446 | MCA445 | SPN548 | SAU516 | MIC EFM073 |
| 16 | 1 | 0.13 | <0.063 | 2 | 1 |
| 18 | 0.5 | <0.063 | <0.063 | 0.25 | 0.25 |
| 20 | 1 | 0.5 | 0.13 | 2 | 2 |

According to a further feature of the invention there is provided a compound of the formula (I), or a pharmaceutically-acceptable salt thereof for use in a method of treatment of the human or animal body by therapy.

We have found that compounds of the present invention inhibit bacterial DNA gyrase and topoisomerase IV and are therefore of interest for their antibacterial effects. In one aspect of the invention the compounds of the invention inhibit bacterial DNA gyrase and are therefore of interest for their antibacterial effects. In one aspect of the invention the compounds of the invention inhibit topoisomerase IV and are therefore of interest for their antibacterial effects. In one aspect of the invention the compounds of the invention inhibit both DNA gyrase and topoisomerase IV and are therefore of interest for their antibacterial effects.

It is expected that the compounds of the present invention will be useful in treating bacterial infections. In one aspect of the invention “infection” or “bacterial infection” refers to a gynecological infection. In one aspect of the invention “infection” or “bacterial infection” refers to a respiratory tract infection (RTI). In one aspect of the invention “infection” or “bacterial infection” refers to a sexually transmitted disease. In one aspect of the invention “infection” or “bacterial infection” refers to a urinary tract infection. In one aspect of the invention “infection” or “bacterial infection” refers to acute exacerbation of chronic bronchitis (ACEB). In one aspect of the invention “infection” or “bacterial infection” refers to acute otitis media. In one aspect of the invention “infection” or “bacterial infection” refers to acute sinusitis. In one aspect of the invention “infection” or “bacterial infection” refers to an infection caused by drug resistant bacteria. In one aspect of the invention “infection” or “bacterial infection” refers to catheter-related sepsis. In one aspect of the invention “infection” or “bacterial infection” refers to chancroid. In one aspect of the invention “infection” or “bacterial infection” refers to chlamydia. In one aspect of the invention “infection” or “bacterial infection” refers to community-acquired pneumonia (CAP). In one aspect of the invention “infection” or “bacterial infection” refers to complicated skin and skin structure infection. In one aspect of the invention “infection” or “bacterial infection” refers to uncomplicated skin and skin structure infection. In one aspect of the invention “infection” or “bacterial infection” refers to endocarditis. In one aspect of the invention “infection” or “bacterial infection” refers to febrile neutropenia. In one aspect of the invention “infection” or “bacterial infection” refers to gonococcal cervicitis. In one aspect of the invention “infection” or “bacterial infection” refers to gonococcal urethritis. In one aspect of the invention “infection” or “bacterial infection” refers to hospital-acquired pneumonia (HAP). In one aspect of the invention “infection” or “bacterial infection” refers to osteomyelitis. In one aspect of the invention “infection” or “bacterial infection” refers to sepsis. In one aspect of the invention “infection” or “bacterial infection” refers to syphilis.

In one aspect of the invention an “infection” or “bacterial infection” refers to an infection caused by Acinetobacter baumanii. In one aspect of the invention an “infection” or “bacterial infection” refers to an infection caused by Acinetobacter haemolyticus. In one aspect of the invention an “infection” or “bacterial infection” refers to an infection caused by Acinetobacter junii. In one aspect of the invention an “infection” or “bacterial infection” refers to an infection caused by Acinetobacter johnsonii. In one aspect of the invention an “infection” or “bacterial infection” refers to an infection caused by Acinetobacter lwoffi. In one aspect of the invention an “infection” or “bacterial infection” refers to an infection caused by Bacteroides bivius. In one aspect of the invention an “infection” or “bacterial infection” refers to an infection caused by Bacteroides fragilis. In one aspect of the invention an “infection” or “bacterial infection” refers to an infection caused by Burkholderia cepacia. In one aspect of the invention an “infection” or “bacterial infection” refers to an infection caused by Campylobacter jejuni. In one aspect of the invention an “infection” or “bacterial infection” refers to an infection caused by Chlamydia pneumoniae. In one aspect of the invention an “infection” or “bacterial infection” refers to an infection caused by Chlamydia urealyticus. In one aspect of the invention an “infection” or “bacterial infection” refers to an infection caused by Chlamydophila pneumoniae. In one aspect of the invention an “infection” or “bacterial infection” refers to an infection caused by Clostridium difficili. In one aspect of the invention an “infection” or “bacterial infection” refers to an infection caused by Enterobacter aerogenes. In one aspect of the invention an “infection” or “bacterial infection” refers to an infection caused by Enterobacter cloacae. In one aspect of the invention an “infection” or “bacterial infection” refers to an infection caused by Enterococcus faecalis. In one aspect of the invention an “infection” or “bacterial infection” refers to an infection caused by Enterococcus faecium. In one aspect of the invention an “infection” or “bacterial infection” refers to an infection caused by Escherichia coli. In one aspect of the invention an “infection” or “bacterial infection” refers to an infection caused by Gardnerella vaginalis. In one aspect of the invention an “infection” or “bacterial infection” refers to an infection caused by Haemophilus parainfluenzae. In one aspect of the invention an “infection” or “bacterial infection” refers to an infection caused by Haemophilus influenzae. In one aspect of the invention an “infection” or “bacterial infection” refers to an infection caused by Helicobacter pylori. In one aspect of the invention an “infection” or “bacterial infection” refers to an infection caused by Klebsiella pneumoniae. In one aspect of the invention an “infection” or “bacterial infection” refers to an infection caused by Legionella pneumophila. In one aspect of the invention an “infection” or “bacterial infection” refers to an infection caused by Methicillin-resistant Staphylococcus aureus. In one aspect of the invention an “infection” or “bacterial infection” refers to an infection caused by Methicillin-susceptible Staphylococcus aureus. In one aspect of the invention an “infection” or “bacterial infection” refers to an infection caused by Moraxella catarrhalis. In one aspect of the invention an “infection” or “bacterial infection” refers to an infection caused by Morganella morganii. In one aspect of the invention an “infection” or “bacterial infection” refers to an infection caused by Mycoplasma pneumoniae. In one aspect of the invention an “infection” or “bacterial infection” refers to an infection caused by Neisseria gonorrhoeae. In one aspect of the invention an “infection” or “bacterial infection” refers to an infection caused by Penicillin-resistant Streptococcus pneumoniae. In one aspect of the invention an “infection” or “bacterial infection” refers to an infection caused by Penicillin-susceptible Streptococcus pneumoniae. In one aspect of the invention an “infection” or “bacterial infection” refers to an infection caused by Peptostreptococcus magnus. In one aspect of the invention an “infection” or “bacterial infection” refers to an infection caused by Peptostreptococcus micros. In one aspect of the invention an “infection” or “bacterial infection” refers to an infection caused by Peptostreptococcus anaerobius. In one aspect of the invention an “infection” or “bacterial infection” refers to an infection caused by Peptostreptococcus asaccharolyticus. In one aspect of the invention an “infection” or “bacterial infection” refers to an infection caused by Peptostreptococcus prevotii. In one aspect of the invention an “infection” or “bacterial infection” refers to an infection caused by Peptostreptococcus tetradius. In one aspect of the invention an “infection” or “bacterial infection” refers to an infection caused by Peptostreptococcus vaginalis. In one aspect of the invention an “infection” or “bacterial infection” refers to an infection caused by Proteus mirabilis. In one aspect of the invention an “infection” or “bacterial infection” refers to an infection caused by Pseudomonas aeruginosa. In one aspect of the invention an “infection” or “bacterial infection” refers to an infection caused by Quinolone-Resistant Staphylococcus aureus. In one aspect of the invention an “infection” or “bacterial infection” refers to an infection caused by Quinolone-Resistant Staphylococcus epidermis. In one aspect of the invention an “infection” or “bacterial infection” refers to an infection caused by Salmonella typhi. In one aspect of the invention an “infection” or “bacterial infection” refers to an infection caused by Salmonella paratyphi. In one aspect of the invention an “infection” or “bacterial infection” refers to an infection caused by Salmonella enteritidis. In one aspect of the invention an “infection” or “bacterial infection” refers to an infection caused by Salmonella typhimurium. In one aspect of the invention an “infection” or “bacterial infection” refers to an infection caused by Serratia marcescens. In one aspect of the invention an “infection” or “bacterial infection” refers to an infection caused by Staphylococcus aureus. In one aspect of the invention an “infection” or “bacterial infection” refers to an infection caused by Staphylococcus epidermidis. In one aspect of the invention an “infection” or “bacterial infection” refers to an infection caused by Staphylococcus saprophyticus. In one aspect of the invention an “infection” or “bacterial infection” refers to an infection caused by Streptoccocus agalactiae. In one aspect of the invention an “infection” or “bacterial infection” refers to an infection caused by Streptococcus pneumoniae. In one aspect of the invention an “infection” or “bacterial infection” refers to an infection caused by Streptococcus pyogenes. In one aspect of the invention an “infection” or “bacterial infection” refers to an infection caused by Stenotrophomonas maltophilia. In one aspect of the invention an “infection” or “bacterial infection” refers to an infection caused by Ureaplasma urealyticum. In one aspect of the invention an “infection” or “bacterial infection” refers to an infection caused by Vancomycin-Resistant Enterococcus faecium. In one aspect of the invention an “infection” or “bacterial infection” refers to an infection caused by Vancomycin-Resistant Enterococcus faecalis. In one aspect of the invention an “infection” or “bacterial infection” refers to an infection caused by Vancomycin-Resistant Staphylococcus aureus. In one aspect of the invention an “infection” or “bacterial infection” refers to an infection caused by Vancomycin-Resistant Staphylococcus epidermis.

In one aspect of the invention an “infection” or “bacterial infection” refers to an infection caused by Acinetobacter spp. In one aspect of the invention an “infection” or “bacterial infection” refers to an infection caused by Bacteroides spp. In one aspect of the invention an “infection” or “bacterial infection” refers to an infection caused by Burkholderia spp. In one aspect of the invention an “infection” or “bacterial infection” refers to an infection caused by Campylobacter spp. In one aspect of the invention an “infection” or “bacterial infection” refers to an infection caused by Chlamydia spp. In one aspect of the invention an “infection” or “bacterial infection” refers to an infection caused by Chlamydophila spp. In one aspect of the invention an “infection” or “bacterial infection” refers to an infection caused by Clostridium spp. In one aspect of the invention an “infection” or “bacterial infection” refers to an infection caused by Enterobacter spp. In one aspect of the invention an “infection” or “bacterial infection” refers to an infection caused by Enterococcus spp. In one aspect of the invention an “infection” or “bacterial infection” refers to an infection caused by Escherichia spp. In one aspect of the invention an “infection” or “bacterial infection” refers to an infection caused by Gardnerella spp. In one aspect of the invention an “infection” or “bacterial infection” refers to an infection caused by Haemophilus spp. In one aspect of the invention an “infection” or “bacterial infection” refers to an infection caused by Helicobacter spp. In one aspect of the invention an “infection” or “bacterial infection” refers to an infection caused by Klebsiella spp. In one aspect of the invention an “infection” or “bacterial infection” refers to an infection caused by Legionella spp. In one aspect of the invention an “infection” or “bacterial infection” refers to an infection caused by Moraxella spp. In one aspect of the invention an “infection” or “bacterial infection” refers to an infection caused by Morganella spp. In one aspect of the invention an “infection” or “bacterial infection” refers to an infection caused by Mycoplasma spp. In one aspect of the invention an “infection” or “bacterial infection” refers to an infection caused by Neisseria spp. In one aspect of the invention an “infection” or “bacterial infection” refers to an infection caused by Peptostreptococcus spp. In one aspect of the invention an “infection” or “bacterial infection” refers to an infection caused by Proteus spp. In one aspect of the invention an “infection” or “bacterial infection” refers to an infection caused by Pseudomonas spp. In one aspect of the invention an “infection” or “bacterial infection” refers to an infection caused by Salmonella spp. In one aspect of the invention an “infection” or “bacterial infection” refers to an infection caused by Serratia spp. In one aspect of the invention an “infection” or “bacterial infection” refers to an infection caused by Staphylococcus spp. In one aspect of the invention an “infection” or “bacterial infection” refers to an infection caused by Streptoccocus spp. In one aspect of the invention an “infection” or “bacterial infection” refers to an infection caused by Stenotrophomonas spp. In one aspect of the invention an “infection” or “bacterial infection” refers to an infection caused by Ureaplasma spp. In one aspect of the invention an “infection” or “bacterial infection” refers to an infection caused by aerobes. In one aspect of the invention an “infection” or “bacterial infection” refers to an infection caused by obligate anaerobes. In one aspect of the invention an “infection” or “bacterial infection” refers to an infection caused by facultative anaerobes. In one aspect of the invention an “infection” or “bacterial infection” refers to an infection caused by gram-positive bacteria. In one aspect of the invention an “infection” or “bacterial infection” refers to an infection caused by gram-negative bacteria. In one aspect of the invention an “infection” or “bacterial infection” refers to an infection caused by gram-variable bacteria. In one aspect of the invention an “infection” or “bacterial infection” refers to an infection caused by atypical respiratory pathogens.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt thereof.

According to a further feature of the invention there is provided a method for inhibition of bacterial DNA gyrase and/or topoisomerase IV in a warm-blooded animal, such as a human being, in need of such treatment which comprises administering to said animal an effective amount of a compound of formula (I) or a pharmaceutically acceptable salt thereof as defined hereinbefore.

According to a further feature of the invention there is provided a method of treating a bacterial infection in a warm-blooded animal, such as a human being, in need of such treatment which comprises administering to said animal an effective amount of a compound of formula (I) or a pharmaceutically acceptable salt thereof as defined hereinbefore.

According to a further feature of the invention there is provided a method of treating a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal, such as a human being, in need of such treatment which comprises administering to said animal an effective amount of a compound of formula (I) or a pharmaceutically acceptable salt thereof as defined hereinbefore.

A further feature of the present invention is a compound of formula (I) and pharmaceutically acceptable salts thereof for use as a medicament. Suitably the medicament is an antibacterial agent.

According to a further aspect of the invention there is provided the use of a compound of formula (I), or a pharmaceutically acceptable salt thereof in the manufacture of a medicament for the production of an anti-bacterial effect in a warm-blooded animal such as a human being.

According to a further aspect of the invention there is provided the use of a compound of formula (I), or a pharmaceutically acceptable salt thereof in the manufacture of a medicament for the inhibition of bacterial DNA gyrase and/or topoisomerase IV in a warm-blooded animal such as a human being.

Thus according to a further aspect of the invention there is provided the use of a compound of formula (I), or a pharmaceutically acceptable salt thereof in the manufacture of a medicament for the treatment of a bacterial infection in a warm-blooded animal such as a human being.

Thus according to a further aspect of the invention there is provided the use of a compound of formula (I), or a pharmaceutically acceptable salt thereof in the manufacture of a medicament for the treatment of a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal such as a human being.

According to a further aspect of the invention there is provided a compound of formula (I), or a pharmaceutically acceptable salt thereof for use in the production of an anti-bacterial effect in a warm-blooded animal such as a human being.

According to a further aspect of the invention there is provided a compound of formula (I), or a pharmaceutically acceptable salt thereof for use in inhibition of bacterial DNA/gyrase and/or topoisomerase IV in a warm-blooded animal such as a human being.

Thus according to a further aspect of the invention there is provided a compound of formula (I), or a pharmaceutically acceptable salt thereof for use in the treatment of a bacterial infection in a warm-blooded animal such as a human being.

Thus according to a further aspect of the invention there is provided a compound of formula (I), or a pharmaceutically acceptable salt thereof for use in the treatment of a bacterial infection selected from a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal such as a human being.

In order to use a compound of the formula (I) or a pharmaceutically-acceptable salt thereof, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition that comprises a compound of the formula (I) or a pharmaceutically-acceptable salt thereof, and a pharmaceutically-acceptable diluent or carrier.

According to a further aspect of the invention there is provided a pharmaceutical composition that comprises a compound of formula (I) as defined hereinbefore or a pharmaceutically acceptable salt thereof, in association with a pharmaceutically acceptable excipient or carrier for use in producing an anti-bacterial effect in an warm-blooded animal, such as a human being.

According to a further aspect of the invention there is provided a pharmaceutical composition that comprises a compound of formula (I) as defined hereinbefore or a pharmaceutically acceptable salt thereof, in association with a pharmaceutically acceptable excipient or carrier for use in inhibition of bacterial DNA gyrase and/or topoisomerase IV in an warm-blooded animal, such as a human being.

According to a further aspect of the invention there is provided a pharmaceutical composition that comprises a compound of formula (I) as defined hereinbefore or a pharmaceutically acceptable salt thereof, in association with a pharmaceutically acceptable excipient or carrier for use in the treatment of a bacterial infection in an warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in the treatment of a gynecological infection, a respiratory tract infection (RTI), a sexually transmitted disease, a urinary tract infection, acute exacerbation of chronic bronchitis (ACEB), acute otitis media, acute sinusitis, an infection caused by drug resistant bacteria, catheter-related sepsis, chancroid, chlamydia, community-acquired pneumonia (CAP), complicated skin and skin structure infection, uncomplicated skin and skin structure infection, endocarditis, febrile neutropenia, gonococcal cervicitis, gonococcal urethritis, hospital-acquired pneumonia (HAP), osteomyelitis, sepsis and/or syphilis in a warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

In addition to the compounds of the present invention the pharmaceutical composition of this invention may also contain or be co-administered (simultaneously, sequentially or separately) with one or more known drugs selected from other clinically useful antibacterial agents (for example, macrolides, quinolones, β-lactams or aminoglycosides) and/or other anti-infective agents (for example, an antifungal triazole or amphotericin). These may include carbapenems, for example meropenem or imipenem, to broaden the therapeutic effectiveness. Compounds of this invention may also contain or be co-administered with bactericidal/permeability-increasing protein (BPI) products or efflux pump inhibitors to improve activity against gram negative bacteria and bacteria resistant to antimicrobial agents.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

In addition to its use in therapeutic medicine, compounds of formula (I) and their pharmaceutically acceptable salts are also useful as pharmacological tools in the development and standardisation of in-vitro and in-vivo test systems for the evaluation of the effects of inhibitors of DNA gyrase in laboratory animals such as cats, dogs, rabbits, monkeys, rats and mice, as part of the search for new therapeutic agents.

In the above other, pharmaceutical composition, process, method, use and medicament manufacture features, the alternative and particular embodiments of the compounds of the invention described herein also apply.

### Combinations

The compounds of the invention described herein may be applied as a sole therapy or may involve, in addition to a compound of the invention, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination. Suitable classes and substances may be selected from one or more of the following:

i) other antibacterial agents for example macrolides e.g. erythromycin, azithromycin or clarithromycin; quinolones e.g. ciprofloxacin or levofloxacin; β-lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or
ii) anti-infective agents for example, an antifungal triazole e.g. or amphotericin; and/or
iii) biological protein therapeutics for example antibodies, cytokines, bactericidal/permeability-increasing protein (BPI) products; and/or
iv) efflux pump inhibitors.

Therefore, in a further aspect of the invention there is provided a compound of the formula (I), or a pharmaceutically acceptable salt thereof and a chemotherapeutic agent selected from:

i) one or more additional antibacterial agents; and/or
ii) one or more anti-infective agents; and/or
iii) biological protein therapeutics for example antibodies, cytokines, bactericidal/permeability-increasing protein (BPI) products; and/or
iv) one or more efflux pump inhibitors.

### EXAMPLES

The invention is now illustrated but not limited by the following Examples in which unless otherwise stated:—

(i) evaporations were carried out by rotary evaporation in-vacuo and work-up procedures were carried out after removal of residual solids by filtration;
(ii) operations were carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable;

(v) the structure of the end-products of the invention were generally confirmed by NMR and mass spectral techniques [proton magnetic resonance spectra is quoted and was generally determined in DMSO-d₆ unless otherwise stated using a Bruker DRX-300 spectrometer operating at a field strength of 300 MHz. Chemical shifts are reported in parts per million downfield from tetramethysilane as an internal standard (8 scale) and peak multiplicities are shown thus: s, singlet; d, doublet; AB or dd, doublet of doublets; dt, doublet of triplets; dm, doublet of multiplets; t, triplet, m, multiplet; br, broad; mass spectral sample data has been collected using a variety of Waters quadrupole mass spectrometer that have been interfaced with Agilent 1100 liquid chromatographs and equipped with SEDEX evaporative light scattering detectors. For ionization, the mass spectrometers have been run in either electrospray ionization (ESI) or atmospheric pressure chemical ionization (APCI) with positive (+) ion and negative (−) ion data being collected. Positive ion data generates a (M+H)⁺response and negative ion data generates a (M−H)⁻ response, and unless otherwise stated, all examples provided are ESI+, reported as the M+H value. Optical rotations were determined at 589 nm at 20° C. using a Perkin Elmer Polarimeter 341, cell pathlength is 10 cm with a 1 mL volume;

(vi) each intermediate was purified to the standard required for the subsequent stage and was characterised in sufficient detail to confirm that the assigned structure was correct; purity was assessed by HPLC, TLC, or NMR and identity was determined by infra-red spectroscopy (IR), mass spectroscopy or NMR spectroscopy as appropriate;
(vii) in which the following abbreviations may be used:—
TSP 3,3′,3″-phosphinidynetris(benzenesulfonic acid), trisodium salt;
TFA trifluoroacetic acid;
EDC 1-(3-dimethylaminopropyl)-3-ethylcarbodiimide hydrochloride;
HOBt 1-hydroxybenzotriazole;
NMM N-methylmorpholine;
HPLC high performance liquid chromatography;
DMF dimethylformamide;
THF tetrahydrofuran;
DIAD diidopropyl azodicarboxylate;
DIEA diisopropylethylamine;
DCM dichloromethane;
HATU O-(7-azabenzotriazol-1-yl)-N,N,N′,N′-tetramethyluronium hexafluorophosphate;
PPTS pyridimium p-toluene sulfonate;
DAST (diethylamino)sulphur trifluoride;
NMP N-methylpyrrolidinone; and
DMSO dimethylsulphoxide.
   (viii) temperatures are quoted as ° C.;
   (ix) Smith Microwave Synthesizer refers to an equipment that uses microwave energy to heat organic reactions in a short period of time; it was used according to the manufacturers instruction and was obtained from Personal Chemistry Uppsala AB;
   (x) Kugelrohr distillation refers to a piece of equipment that distils liquids and heats sensitive compounds using air-bath oven temperature; it was used according to the manufacturers instruction and was obtained from Buchi, Switzerland or Aldrich, Milwaukee, USA;
   (xi) Where cis(±) or trans(±) is used it is to be understood that this refers to a racemic mixture of the cis or the trans isomers, (−) or (+) refers to the single enantiomer as does R,R or S,S where quoted; Where “rel” is used this describes the relative relationship of the chiral centers e.g. Examples 49 and 50, i.e. the absolute configuration of the stereochemistry has not been determined; and
   (xii) GCMS is Gas phase chromatography (model 6890N) with Mass Spectrometer(model 5973) manufactured by Agilent and was used according to manufacturers instructions.

### Example 1

### Methyl 2-(10-{[(3,4-dichloro-5-methyl-1H-pyrrol-2-yl)carbonyl]amino}-1,4-dioxa-7-azaspiro[4.5]dec-7-yl)-1,3-thiazole-5-carboxylate

To a suspension/solution of 3,4-dichloro-N-1,4-dioxa-7-azaspiro[4.5]dec-10-yl-5-methyl-1H-pyrrole-2-carboxamide (0.10 g, 0.3 mmol, Intermediate 17) and methyl 2-bromo-1,3-thiazole-5-carboxylate (0.066 g, 0.3 mmol) in NMP (2 mL) was added DIEA (0.10 mL, 0.60 mmol). After heating in the microwave reactor at 80° for 30 minutes the reaction was complete. The crude reaction mixture was cooled to room temperature and then slowly poured into water. After stirring at room temperature for several minutes the resulting precipitate was filtered, washed with water, and dried overnight (0.096 g). MS (ES) (M+H)⁺: 475 for C₁₈H₂₀Cl₂N₄O₅S; NMR: 1.69 (d, 1H), 1.93 (s, 1H), 2.18 (s, 3H), 3.30-3.42 (m, 4H), 3.75 (s, 3H), 3.77-3.86 (m, 1H), 3.90-4.02 (m, 2H), 4.02-4.09 (m, 1H), 4.09-4.21 (m, 2H), 4.35-4.48 (m, 1H), 6.95 (d, 1H), 7.83 (s, 1H), 12.17 (s, 1H).

### Examples 2-10

The following Examples were prepared by the procedure described in Example 1 from the starting materials (SM) indicated.

| Ex | Compound | Data | SM |
|---|---|---|---|
| 2 | Methyl 4-acetyl-2-(10- | MS (ES) (M + H)⁺: 517 for | Intermediate 17 |
| | {[(3,4-dichloro-5- | C₂₀H₂₂Cl₂N₄O₆S | and methyl 4- |
| | methyl-1H-pyrrol-2- | NMR: 1.72 (s, 1 H), 1.95 (s, 1 H), | acetyl-2-chloro- |
| | yl)carbonyl]amino}- | 2.18 (s, 3 H), 2.46 (s, 3 H), 3.47 (s, 2 H), | 1,3-thiazole-5- |
| | 1,4-dioxa-7- | 3.73 (s, 3 H), 3.80 (d, J = 7.72 Hz, 1 H), | carboxylate |
| | azaspiro[4.5]dec-7-yl)- | 3.96 (d, J = 6.97 Hz, 3 H), 4.10 (d, J = 6.97 Hz, | |
| | 1,3-thiazole-5- | 2 H), 4.44 (s, 1 H), 6.95 (d, J = 9.04 Hz, | |
| | carboxylate | 1 H), 12.17 (s, 1 H) | |
| 3 | Isopropyl 2-(10-{[(3,4- | MS (ES) (M + H)⁺: 560 for | Intermediate 17 |
| | dichloro-5-methyl-1H- | C₂₂H₂₇Cl₂N₅O₆S | and |
| | pyrrol-2- | NMR: 1.21 (d, 6 H), 1.67 (d, 1 H), | Intermediate 31 |
| | yl)carbonyl]amino}- | 1.84-2.00 (m, 2 H), 2.12-2.22 (m, 3 H), | |
| | 1,4-dioxa-7- | 2.64-2.73 (m, 3 H), 3.26-3.34 (m, 1 | |
| | azaspiro[4.5]dec-7-yl)- | H), 3.74-3.90 (m, 1 H), 3.94 (s, 2 H), | |
| | 4-[(methylamino)carbonyl]- | 4.03-4.18 (m, 3 H), 4.42 (s, 1 H), | |
| | 1,3-thiazole- | 4.90-5.04 (m, 1 H), 6.93 (d, 1 H), 8.33 (d, 1 | |
| | 5-carboxylate | H), 12.18 (s, 1 H) | |
| 4 | Isopropyl 2-(10-{[(3,4- | MS (ES) (M + H)⁺: 497 for | Intermediate 17 |
| | dichloro-5-methyl-1H- | C₂₂H₂₆Cl₂N₄O₅ | and |
| | pyrrol-2- | NMR: 1.27-1.36 (m, 6 H), 1.62 (s, 1 | Intermediate 54 |
| | yl)carbonyl]amino}- | H), 1.90 (s, 1 H), 2.18 (s, 3 H), 3.00 (d, | |
| | 1,4-dioxa-7- | 1 H), 3.12 (s, 1 H), 3.79 (s, 1 H), | |
| | azaspiro[4.5]dec-7- | 3.91 (s, 1 H), 4.09 (s, 2 H), 4.35 (s, 2 H), | |
| | yl)isonicotinate | 5.07-5.18 (m, 1 H), 6.89-7.00 (m, 2 | |
| | | H), 7.24 (s, 1 H), 8.22 (d, 1 H), 12.15 (s, | |
| | | 1 H) | |
| 5 | Ethyl 2-(10-{[(3,4- | MS (ES) (M + H)⁺: 590 for | Intermediate 17 |
| | dichloro-5-methyl-1H- | C₂₃H₂₉Cl₂N₅O₇S | and |
| | pyrrol-2-yl)carbonyl]amino}- | NMR: 1.21 (t, 3 H), 1.69 (s, 1 H), | Intermediate 20 |
| | 1,4-dioxa-7- | 1.93 (s, 2 H), 2.18 (s, 3 H), 3.26 (s, 3 H), | |
| | azaspiro[4.5]dec-7-yl)- | 3.29-3.36 (m, 2 H), 3.41 (d, 2 H), | |
| | 4-{[(2-methoxyethyl)amino]carbonyl}- | 3.82 (s, 2 H), 3.95 (s, 2 H), 4.16 (d, 2 H), | |
| | 1,3- | 4.42 (s, 1 H), 6.95 (s, 1 H), 8.49 (s, 1 | |
| | thiazole-5-carboxylate | H), 12.17 (s, 1 H) | |
| 6 | Methyl 2-(11-{[(3,4- | MS (ES) (M + H)⁺: 489 for | Intermediate 18 |
| | dichloro-5-methyl-1H- | C₁₉H₂₂Cl₂N₄O₅S | and methyl 2- |
| | pyrrol-2-yl)carbonyl]amino}- | NMR: 1.50 (d, 1 H), 1.67 (s, 1 H), | bromo-1,3- |
| | 1,5-dioxa-8- | 1.89 (s, 2 H), 2.19 (s, 3 H), 3.17 (d, 1 H), | thiazole-5- |
| | azaspiro[5.5]undec-8- | 3.31 (s, 3 H), 3.49 (s, 1 H), 3.75 (s, 3 | carboxylate |
| | yl)-1,3-thiazole-5- | H), 3.84 (s, 2 H), 4.07 (s, 2 H), 4.15 (d, | |
| | carboxylate | 2 H), 5.11 (s, 1 H), 7.17 (d, 1 H), | |
| | | 7.86 (s, 1 H) | |
| 7 | Ethyl 2-(11-{[(3,4- | MS (ES) (M + H)⁺: 604 for | Intermediate 18 |
| | dichloro-5-methyl-1H- | C₂₄H₃₁Cl₂N₅O₇S | and |
| | pyrrol-2-yl)carbonyl]amino}- | NMR: 1.21 (t, 3 H), 1.50 (s, 1 H), | Intermediate 20 |
| | 1,5-dioxa-8- | 1.65 (s, 1 H), 1.87 (s, 1 H), 2.19 (s, 3 H), | |
| | azaspiro[5.5]undec-8- | 3.18 (s, 2 H), 3.26 (s, 4 H), 3.42 (t, 4 H), | |
| | yl)-4-{[(2- | 3.82 (d, 3 H), 3.85 (s, 2 H), 4.16 (q, 3 | |
| | methoxyethyl)amino]carbonyl}- | H), 7.18 (d, 1 H), 8.50 (t, 1 H), 12.17 (s, | |
| | 1,3- | 1 H) | |
| | thiazole-5-carboxylate | | |
| 8 | Isopropyl 2-(11-{[(3,4- | MS (ES) (M + H)⁺: 574 for | Intermediate 18 |
| | dichloro-5-methyl-1H- | C₂₃H₂₉Cl₂N₅O₆S | and |
| | pyrrol-2-yl)carbonyl]amino}- | NMR: 1.21 (d, 6 H), 1.50 (s, 1 H), | Intermediate 31 |
| | 1,5-dioxa-8- | 1.64 (s, 1 H), 1.87 (s, 2 H), 2.18 (s, 3 H), | |
| | azaspiro[5.5]undec-8- | 2.69 (d, 3 H), 3.16 (d, 1 H), 3.32 (d, 3 | |
| | yl)-4-[(methylamino)carbonyl]- | H), 3.48 (d, 1 H), 3.80 (s, 3 H), 4.10 (s, | |
| | 1,3-thiazole- | 3 H), 4.98 (d, 1 H), 7.17 (d, 1 H), | |
| | 5-carboxylate | 8.31-8.38 (m, 1 H) | |
| 9 | Methyl 4-acetyl-2-(11- | MS (ES) (M + H)⁺: 531 for | Intermediate 18 |
| | {[(3,4-dichloro-5- | C₂₁H₂₄Cl₂N₄O₆S | and methyl 4- |
| | methyl-1H-pyrrol-2- | NMR: 1.51 (s, 1 H), 1.64 (d, 1 H), | acetyl-2-chloro- |
| | yl)carbonyl]amino}- | 1.89 (s, 1 H), 2.19 (s, 3 H), 2.47 (s, 3 H), | 1,3-thiazole-5- |
| | 1,5-dioxa-8- | 2.89-3.03 (m, 1 H), 3.20 (d, 1 H), | carboxylate |
| | azaspiro[5.5]undec-8- | 3.31 (s, 3 H), 3.51 (s, 1 H), 3.72 (s, 3 H), | |
| | yl)-1,3-thiazole-5- | 3.79 (s, 1 H), 3.89 (s, 1 H), 4.07 (s, 1 | |
| | carboxylate | H), 4.12-4.21 (m, 1 H), 5.00 (s, 1 H), | |
| | | 7.18 (d, 1 H) | |
| 10 | Methyl 2-(4-{[(3,4- | MS (ES) (M + H)⁺: 477 for | Intermediate 19 |
| | dichloro-5-methyl-1H- | C₁₈H₂₂Cl₂N₄O₅S | and methyl 2- |
| | pyrrol-2-yl)carbonyl]amino}- | NMR: 1.94 (m, 2 H), 2.27 (s, 3 H), | bromo-1,3- |
| | 3,3- | 3.22 (s, 6 H), 3.64 (m, 2 H), 3.75 (s, 3 H), | thiazole-5- |
| | dimethoxypiperidin-1- | 3.93 (m, 1 H), 4.38 (m, 1 H), 7.25 (d, 1 | carboxylate |
| | yl)-1,3-thiazole-5- | H), 7.90 (s, 1 H), 12.22 (s, 1 H) | |
| | carboxylate | | |

### Example 11

### 2-(10-{[(3,4-Dichloro-5-methyl-1H-pyrrol-2-yl)carbonyl]amino}-1,4-dioxa-7-azaspiro[4.5]dec-7-yl)-1,3-thiazole-5-carboxylic acid

Barium hydroxide (0.080 g, 0.47 mmol) was added to a suspension of methyl 2-(10-{[(3,4-dichloro-5-methyl-1H-pyrrol-2-yl)carbonyl]amino}-1,4-dioxa-7-azaspiro[4.5]dec-7-yl)-1,3-thiazole-5-carboxylate (Example 1, 0.074 g, 0.15 mmol) in methanol (2 mL) and water (0.5 mL). After heating at an external temperature of 60° C. for 2 hours, the reaction mixture was cooled to room temperature and diluted with water. After acidifying with 1N HCl, mixture was extracted with EtOAc (×3), which was then dried with MgSO₄ and concentrated to a tan solid. MS (ES) (M+H)⁺: 461 for C₁₇H₁₈Cl₂N₄O₅S; NMR: 1.72 (s, 1H), 1.91 (s, 1H), 2.18 (s, 3H), 3.31-3.43 (m, 4H), 3.80 (d, 1H), 4.00 (d, 2H), 4.06-4.16 (m, 1H), 4.33-4.48 (m, 1H), 6.96 (d, 1H), 7.73 (s, 1H), 12.17 (s, 1H), 12.62 (s, 1H).

### Examples 12-20

The following Examples were prepared by the procedure described in Example 11 from the starting materials (SM) indicated.

| Ex | Compound | Data | SM |
|---|---|---|---|
| 12 | 4-Acetyl-2-(10-{[(3,4- | MS (ES) (M + H)⁺: 503 for | Example 2 |
| | dichloro-5-methyl-1H-pyrrol- | C₁₉H₂₀Cl₂N₄O₆S | |
| | 2-yl)carbonyl]amino}-1,4- | NMR: 1.72 (s, 1 H), 1.94 (s, 1 H), 2.18 (s, | |
| | dioxa-7-azaspiro[4.5]dec-7- | 3 H), 3.34 (s, 4 H), 3.73-3.88 (m, 2 H), | |
| | yl)-1,3-thiazole-5-carboxylic | 3.97 (s, 3 H), 4.13 (s, 2 H), 4.43 (s, 1 H), | |
| | acid | 6.94 (s, 1 H), 12.17 (s, 1 H), 13.24 (s, 1 H) | |
| 13 | 2-(10-{[(3,4-Dichloro-5- | MS (ES) (M + H)⁺: 518 for | Example 3 |
| | methyl-1H-pyrrol-2- | C₁₉H₂₁Cl₂N₅O₆S | |
| | yl)carbonyl]amino}-1,4- | NMR: 1.73 (s, 1 H), 1.95 (s, 1 H), 2.18 (s, | |
| | dioxa-7-azaspiro[4.5]dec-7- | 3 H), 3.26 (s, 4 H), 3.50 (s, 3 H), 3.79 (d, | |
| | yl)-4- | 1 H), 4.02 (s, 1 H), 4.13 (s, 2 H), 4.44 (s, 1 | |
| | [(methylamino)carbonyl]-1,3- | H), 6.96 (d, 1 H), 9.41 (s, 1 H), 12.17 (s, 1 | |
| | thiazole-5-carboxylic acid | H) | |
| 14 | 2-(10-{[(3,4-Dichloro-5- | MS (ES) (M + H)⁺: 562 for | Example 5 |
| | methyl-1H-pyrrol-2- | C₂₁H₂₅Cl₂N₅O₇S | |
| | yl)carbonyl]amino}-1,4- | NMR: 1.73 (s, 1 H), 1.94 (s, 1 H), 2.18 (s, | |
| | dioxa-7-azaspiro[4.5]dec-7- | 3 H), 3.26 (s, 3 H), 3.34 (s, 5 H), 3.50 (s, 4 | |
| | yl)-4-{[(2-methoxyethyl)amino]carbonyl}- | H), 3.79 (d, 1 H), 4.02 (s, 1 H), | |
| | 1,3-thiazole- | 4.08-4.19 (m, 2 H), 4.35-4.49 (m, 1 H), 6.96 (d, 1 | |
| | 5-carboxylic acid | H), 9.41 (s, 1 H), 12.17 (s, 1 H) | |
| 15 | 2-(10-{[(3,4-Dichloro-5- | MS (ES) (M + H)⁺: 455 for C₁₉H₂₀Cl₂N₄O₅ | Example 4 |
| | methyl-1H-pyrrol-2- | NMR: 1.65 (d, 1 H), 1.92 (d, 1 H), 2.18 (s, | |
| | yl)carbonyl]amino}-1,4- | 3 H), 3.11 (s, 2 H), 3.81 (t, 1 H), 3.93 (d, 1 | |
| | dioxa-7-azaspiro[4.5]dec-7- | H), 4.10 (d, 2 H), 4.37 (s, 2 H), 4.51 (s, 1 | |
| | yl)isonicotinic acid | H), 6.94 (d, 1 H), 7.04 (d, 1 H), 7.38 (s, 1 | |
| | | H), 8.19 (d, 1 H), 12.17 (s, 1 H) | |
| 16 | 2-(11-{[(3,4-Dichloro-5- | MS (ES) (M + H)⁺: 475 for | Example 6 |
| | methyl-1H-pyrrol-2- | C₁₈H₂₀Cl₂N₄O₅S | |
| | yl)carbonyl]amino}-1,5- | NMR: 1.51 (s, 2 H), 1.67 (s, 1 H), 1.88 (s, | |
| | dioxa-8-azaspiro[5.5]undec-8- | 2 H), 2.19 (s, 3 H), 3.17 (s, 1 H), 3.47 (s, 2 | |
| | yl)-1,3-thiazole-5-carboxylic | H), 3.83 (s, 4 H), 4.11 (s, 3 H), 5.12 (s, 1 | |
| | acid | H), 7.17 (s, 1 H), 7.77 (s, 1 H), 12.17 (s, 1 | |
| | | H) | |
| 17 | 2-(11-{[(3,4-Dichloro-5- | MS (ES) (M + H)⁺: 576 for | Example 7 |
| | methyl-1H-pyrrol-2- | C₂₂H₂₇Cl₂N₅O₇S | |
| | yl)carbonyl]amino}-1,5- | NMR: 1.30-1.46 (m, 1 H), 1.58 (d, 1 H), | |
| | dioxa-8-azaspiro[5.5]undec-8- | 1.84 (s, 2 H), 2.12 (s, 3 H), 3.13 (d, 1 H), | |
| | yl)-4-{[(2-methoxyethyl)amino]carbonyl}- | 3.19 (s, 3 H), 3.29 (s, 4 H), 3.37-3.51 (m, | |
| | 1,3-thiazole- | 3 H), 3.77 (s, 2 H), 4.10 (d, 3 H), 7.12 (d, | |
| | 5-carboxylic acid | 1 H), 9.38 (s, 1 H), 12.10 (s, 1 H) | |
| 18 | 2-(11-{[(3,4-Dichloro-5- | MS (ES) (M + H)⁺: 532 for | Example 8 |
| | methyl-1H-pyrrol-2- | C₂₀H₂₃Cl₂N₅O₆S | |
| | yl)carbonyl]amino}-1,5- | NMR: 1.39-1.52 (m, 1 H), 1.67 (s, 1 H), | |
| | dioxa-8-azaspiro[5.5]undec-8- | 1.89 (d, J = 9.80 Hz, 2 H), 2.19 (s, 3 H), | |
| | yl)-4- | 2.90 (d, J = 4.71 Hz, 2 H), 3.20 (s, 1 H), | |
| | [(methylamino)carbonyl]-1,3- | 3.36 (s, 3 H), 3.84 (s, 2 H), 4.15 (d, | |
| | thiazole-5-carboxylic acid | J = 11.87 Hz, 3 H), 7.18 (d, J = 8.10 Hz, 1 | |
| | | H), 9.44 (s, 1 H), 12.17 (s, 1 H) | |
| 19 | 4-Acetyl-2-(11-{[(3,4- | MS (ES) (M + H)⁺: 517 for | Example 9 |
| | dichloro-5-methyl-1H-pyrrol- | C₂₀H₂₂Cl₂N₄O₆S | |
| | 2-yl)carbonyl]amino}-1,5- | NMR: 1.51 (s, 1 H), 1.68 (s, 1 H), 1.88 (s, | |
| | dioxa-8-azaspiro[5.5]undec-8- | 2 H), 2.19 (s, 3 H), 2.48 (s, 3 H), 3.18 (s, 2 | |
| | yl)-1,3-thiazole-5-carboxylic | H), 3.50 (s, 1 H), 3.79 (s, 3 H), 3.89 (s, 1 | |
| | acid | H), 4.13 (s, 1 H), 5.01 (s, 1 H), 7.18 (d, 1 | |
| | | H), 12.17 (s, 1 H) | |
| 20 | 2-(4-{[(3,4-Dichloro-5- | MS (ES) (M − H)⁻: 461 for C₁₇H₂₀Cl₂N₄O₅S | Example |
| | methyl-1H-pyrrol-2- | NMR: 1.96 (m, 2 H), 2.25 (s, 3 H), | 10 |
| | yl)carbonyl]amino}-3,3- | 3.23 (s, 6 H), 3.56 (m, 2 H), 3.74 (d, 1 H), | |
| | dimethoxypiperidin-1-yl)-1,3- | 3.97 (d, 1 H), 4.93 (m, 1 H), 7.25 (d, 1 H), | |
| | thiazole-5-carboxylic acid | 7.90 (s, 1 H), 12.28 (s, 1 H) | |

### Example 21

### Sodium 2-(10-{[(3,4-dichloro-5-methyl-1H-pyrrol-2-yl)carbonyl]amino}-1,4-dioxa-7-azaspiro[4.5]dec-7-yl)-4-[(methylamino)carbonyl]-1,3-thiazole-5-carboxylate

To a suspension of 2-(10-{[(3,4-dichloro-5-methyl-1H-pyrrol-2-yl)carbonyl]amino}-1,4-dioxa-7-azaspiro[4.5]dec-7-yl)-4-[(methylamino)carbonyl]-1,3-thiazole-5-carboxylic acid (0.027 g, 0.052 mmol, Example 13) in 1:1 methanol:water (5 mL) was added aqueous 0.1N sodium hydroxide (0.52 mL, 0.052 mmol). Once the mixture was homogeneous, the reaction was concentrated and dried (0.026 g). MS (ES) (M+H)⁺: 518 for C₁₉H₂₀Cl₂N₅O₆SNa NMR: 1.70 (s, 1H), 1.86 (s, 1H), 2.14 (s, 3H), 2.68 (d, 3H), 3.06-3.22 (m, 2H), 3.80 (s, 2H), 3.97 (s, 2H), 4.05 (s, 2H), 4.30 (s, 1H), 6.98 (d, 1H), 12.16 (s, 1H), 13.05 (s, 1H).

### Example 22

The following Example was prepared by the procedure described in Example 21 from the starting materials (SM) indicated.

| Ex | Compound | Data | SM |
|---|---|---|---|
| 22 | Sodium 2-(10-{[(3,4- | MS (ES) (M + H)⁺: 562 for C₂₁H₂₅Cl₂N₅O₇S | Example |
| | dichloro-5-methyl-1H- | NMR: 1.71 (s, 1 H), 1.90 (d, 1 H), 2.18 (s, | 14 |
| | pyrrol-2-yl)carbonyl]amino}- | 3 H), 3.07-3.21 (m, 2 H), 3.24 (s, 3 H), | |
| | 1,4-dioxa-7- | 3.28-3.32 (m, 1 H), 3.34 (s, 2 H), 3.39 (t, | |
| | azaspiro[4.5]dec-7-yl)-4- | 3 H), 3.80 (t, 2 H), 3.89-4.03 (m, 2 H), | |
| | {[(2-methoxyethyl)amino]carbonyl}- | 4.07 (dd, 1 H), 4.14 (s, 1 H), 4.32 (d, 1 H), | |
| | 1,3-thiazole-5- | 6.97 (d, 1 H), 12.17 (s, 1 H), 13.24 (s, 1 H) | |
| | carboxylate | | |

### Example 23

### 2-[(3E)-4-{[(3,4-Dichloro-5-methyl-1H-pyrrol-2-yl)carbonyl]amino}-3-(methoxyimino)piperidin-1-yl]-4-{[(2-methoxyethyl)amino]carbonyl}-1,3-thiazole-5-carboxylic acid

A solution of 82 mg (0.14 mmol) of allyl 2-[(3E)-4-{[(3,4-dichloro-5-methyl-1H-pyrrol-2-yl)carbonyl]amino}-3-(methoxyimino)piperidin-1-yl]-4-{[(2-methoxyethyl)amino]carbonyl}-1,3-thiazole-5-carboxylate (Example 30), 2 mg Pd(OAc)₂ (9 μmol), 10 mg (18 μmol) TSP and 34 μL (0.32 mmol) Et₂NH in 3 ml CH₃CN, 1 mL MeOH and 0.5 mL water was stirred at room temperature for 60 min. Solvent was removed and the residue was taken up in water along with 260 μl, 1N HCl. Extraction twice with EtOAc was followed by washing of the extracts with water and brine. The extracts were dried (MgSO₄) and concentrated to give a solid that was triturated with EtOH to afford 31 mg of product. MS (ES) (M+H)⁺: 547 for C₂₀H₂₄Cl₂N₆O₆S; NMR: 1.81 (m, 1H), 1.94 (m, 1H), 2.23 (s, 3H), 2.41 (m, 1H), 3.33 (s, 3H), 3.58 (s, 3H), 3.64 (m, 1H), 3.97 (s, 3H), 4.02 (m, 1H), 4.91 (m, 2H), 7.76 (m, 1H), 9.49 (s, 1H), 12.16 (s, 1H), 16.42 (s, 1H). The product also contained some (<5%) of the corresponding (Z) isomer.

### Example 24

The following Examples were prepared by the procedure described in Example 23 from the reagent indicated.

| Ex | Compound | Data | Reagent |
|---|---|---|---|
| 24¹ | 4-(Aminocarbonyl)-2-[(3E)-4-{[(3,4- | MS (ES) (M + H)⁺: 489 for | Example |
| | dichloro-5-methyl-1H-pyrrol-2- | C₁₇H₁₈Cl₂N₆O₅S; NMR: 1.94 (m, | 35 |
| | yl)carbonyl]amino}-3- | 1 H), 2.23 (s, 3 H), 2.35 (m, 1 H), | |
| | (methoxyimino)piperidin-1-yl]-1,3- | 3.61 (m, 1 H), 3.92 (s, 3 H), | |
| | thiazole-5-carboxylic acid | 4.22 (d, 1 H), 4.87 (m, 2 H), 7.65 (d, 1 | |
| | | H), 7.88-9.03 (m, 2 H), 12.14 (s, | |
| | | 1 H). | |

¹The product also contained some (<5%) of the corresponding (Z) isomer.

### Example 25

### 2-[(3E)-4-{[(3,4-Dichloro-5-methyl-1H-pyrrol-2-yl)carbonyl]amino}-3-(methoxyimino)piperidin-1-yl]-4-(1-methyl-1H-1,2,4-triazol-5-yl)-1,3-thiazole-5-carboxylic acid

A mixture of 70 mg (0.13 mmol) methyl 2-[(3E)-4-{[(3,4-dichloro-5-methyl-1H-pyrrol-2-yl)carbonyl]amino}-3-(methoxyimino)piperidin-1-yl]-4-(1-methyl-1H-1,2,4-triazol-5-yl)-1,3-thiazole-5-carboxylate (Example 34) and LiI (100 mg, 0.75 mmol) in 30 ml THF was heated at reflux overnight with a 20 ml Dean-Stark trap removing 20 ml of solvent. Solvent was removed and the residue was dissolved in water. 1N HCl was added to bring the pH to about 5. The mixture was extracted twice with EtOAc, each extract being washed with brine. Drying (MgSO₄) and removal of solvent gave a solid that was triturated with MeOH and dried in vacuo to afford 48 mg of product. MS (ES) (M+H)⁺: 527 for C₁₉H₂₀Cl₂N₈O₄S; NMR: 1.92 (m, 1H), 2.24 (s, 3H), 2.37 (m, 1H), 2.94 (d, 3H), 3.55 (m, 1H), 3.91 (s, 3H), 4.08 (s, 3H), 4.15 (d, 1H), 4.93 (m, 1H), 5.12 (d, 1H), 7.71 (d, 1H), 8.25 (s, 1H), 12.14 (s, 1H). The product also contained some (<5%) of the corresponding (Z) isomer.

### Examples 26-27

The following Examples were prepared by the procedure described in Example 25 from the reagent indicated.

| Ex | Compound | Data | Reagent |
|---|---|---|---|
| 26¹ | 2-[(3E)-4-{[(3,4-Dichloro-5- | MS (ES) (M + H)⁺: 503 for | Example |
| | methyl-1H-pyrrol-2- | C₁₈H₂₀Cl₂N₆O₅S; NMR: 1.77 (m, 1 | 31 |
| | yl)carbonyl]amino}-3- | H), 2.21 (s, 3 H), 2.43 (m, 1 H), | |
| | (methoxyimino)piperidin-1-yl]-4- | 2.91 (d, 3 H), 3.34 (s, 4H), 3.52 (s, 3 H), | |
| | [(methylamino)carbonyl]-1,3- | 3.87 (s, 3 H), 4.03 (m, 1 H), 4.33 (m, 2 | |
| | thiazole-5-carboxylic acid | H), 4.82 (m, 2 H), 7.71 (m, 1 H), | |
| | | 9.43 (s, 1 H), 12.14 (s, 1 H). | |
| 27¹ | 2-[(3E)-4-{[(3,4-Dichloro-5- | MS (ES) (M + H)⁺: 570 for | Example |
| | methyl-1H-pyrrol-2- | C₂₂H₂₅Cl₂N₇O₅S; NMR: 1.88 (m, 1 | 36 |
| | yl)carbonyl]amino}-3- | H), 2.23 (s, 3 H), 2.32-2.41 (m, 1 H), | |
| | (methoxyimino)piperidin-1-yl]-4- | 3.22 (s, 3 H), 3.61 (m, 1 H), 3.68 (m, 2 | |
| | [1-(2-methoxyethyl)-1H- | H), 3.91 (s, 3 H), 4.10 (m, 1 H), | |
| | imidazol-2-yl]-1,3-thiazole-5- | 4.82 (m, 2 H), 4.88 (m, 1 H), 5.12 (d, 1 H), | |
| | carboxylic acid | 7.41 (s, 1 H), 7.48 (s, 1 H), 7.72 (d, 2 | |
| | | H), 12.12 (s, 1 H). | |

¹The product also contained some (<5%) of the corresponding (Z) isomer.

### Example 28

### 2-[(3E)-4-{[(3,4-Dichloro-5-methyl-1H-pyrrol-2-yl)carbonyl]amino}-3-(methoxyimino)piperidin-1-yl]-4-[1-(methoxymethyl)-1H-imidazol-2-yl]-1,3-thiazole-5-carboxylic acid

A mixture of 110 mg (0.19 mmol) methyl 2-[(3E)-4-{[(3,4-dichloro-5-methyl-1H-pyrrol-2-yl)carbonyl]amino}-3-(methoxyimino)piperidin-1-yl]-4-[1-(methoxymethyl)-1H-imidazol-2-yl]-1,3-thiazole-5-carboxylate (Example 37) and LiI (100 mg, 0.75 mmol) in 30 ml THF was heated at 85° C. (external temperature) for 2 days. The mixture was diluted in water and 1N HCl was added to bring the pH to about 7. The mixture was extracted twice with EtOAc, each extract being washed with brine. Drying (MgSO₄) and removal of solvent was followed by purification by reverse phase HPLC (30-65% CH₃CN in H₂O gradient with 0.1% TFA) to afford 16 mg of product. MS (ES) (M+H)⁺: 556 for C₂₁H₂₃Cl₂N₇O₅S; NMR: 1.91 (m, 1H), 2.21 (s, 3H), 2.35 (m, 1H), 3.32 (s, 3H), 3.48 (m, 1H), 3.91 (s, 3H), 4.14 (d, 1H), 4.92 (m, 1H), 5.10 (m, 1H), 6.03 (s, 1H), 7.41 (m, 1H), 7.69 (s, 2H), 12.10 (s, 1H). The product also contained some (<5%) of the corresponding (Z) isomer.

### Example 29

### 2-[(3E)-4-{[(3,4-Dichloro-5-methyl-1H-pyrrol-2-yl)carbonyl]amino}-3-(methoxyimino)piperidin-1-yl]-4-(1H-imidazol-2-yl)-1,3-thiazole-5-carboxylic acid

A mixture of 84 mg (0.15 mmol) methyl 2-[(3E)-4-{[(3,4-dichloro-5-methyl-1H-pyrrol-2-yl)carbonyl]amino}-3-(methoxyimino)piperidin-1-yl]-4-[1-(methoxymethyl)-1H-imidazol-2-yl]-1,3-thiazole-5-carboxylate (Example 37) and LiI (80 mg, 0.59 mmol) in 30 ml THF was heated at reflux overnight with a 20 ml Dean-Stark trap removing 20 ml of solvent. Additional (60 mg) LiI was added and the mixture was heated at reflux for 24 hours. The mixture was diluted in water and 1N HCl was added to bring the pH to about 7. The mixture was extracted twice with EtOAc, each extract being washed with brine. Drying (MgSO₄) and removal of solvent was followed by purification by reverse phase HPLC (20-75% CH₃CN in H₂O gradient with 0.1% TFA) to afford 12 mg of product. MS (ES) (M+H)⁺: 512 for C₁₉H₁₉Cl₂N₇O₄S; NMR: 1.92 (m, 1H), 2.24 (s, 3H), 2.39 (m, 1H), 3.61 (m, 1H), 3.87 (s, 3H), 3.82-4.01 (m, 1H), 4.25 (d, 1H), 4.91 (m, 1H), 5.01 (d, 1H), 7.94 (s, 2H), 7.74 (d, 1H), 12.15 (s, 1H). The product also contained some (<5%) of the corresponding (Z) isomer.

### Example 30

### Allyl 2-[(3E)-4-{[(3,4-dichloro-5-methyl-1H-pyrrol-2-yl)carbonyl]amino}-3-(methoxy imino)piperidin-1-yl]-4-{[(2-methoxyethyl)amino]carbonyl}-1,3-thiazole-5-carboxylate

A mixture of 100 mg (0.23 mmol) of 3,4-dichloro-N-[(3E)-3-(methoxy imino)piperidin-4-yl]-5-methyl-1H-pyrrole-2-carboxamide trifluoroacetate salt (Intermediate 21) 85 mg of allyl 2-chloro-4-{[(2-methoxyethyl)amino]carbonyl}-1,3-thiazole-5-carboxylate (Intermediate 29) and 50 mg (0.60 mmol) NaHCO₃ in 2 mL DMF was heated at 80° C. for 60 min. The mixture was poured into dilute HCl. Solids were filtered, washed with water and dried in vacuo. The solids were chromatographed on silica gel (100% DCM with gradient elution to 100% EtOAc) to afford 95 mg of product as a solid. MS (ES) (M+H)⁺: 587 for C₂₃H₂₈Cl₂N₆O₆S; NMR: 1.91 (m, 2H), 2.23 (s, 3H), 2.31 (m, 1H), 3.22 (s, 3H), 3.36 (m, 3H), 3.42 (m, 2H), 3.68 (m, 1H), 3.84 (m, 1H), 3.98 (s, 3H), 4.18 (d, 1H), 4.84-5.07 (m, 2 H), 5.23-5.43 (m, 2H), 5.83-5.60 (m, 1H), 7.76 (d, 1H), 8.51 (s, 1H), 12.11 (s, 1H). The product also contained some (<5%) of the corresponding (Z) isomer.

### Examples 31-37

The following Examples were prepared by the procedure described in Example 30 from 3,4-dichloro-N-[(3E)-3-(methoxyimino)piperidin-4-yl]-5-methyl-1H-pyrrole-2-carboxamide trifluoroacetate salt (Intermediate 21) and the reagent indicated. The products also contained some (<5%) of the corresponding (Z) isomer.

| Ex | Compound | Data | Reagent |
|---|---|---|---|
| 31 | Methyl 2-[(3E)-4-{[(3,4- | MS (ES) (M + H)⁺: 517 for | Intermediate |
| | dichloro-5-methyl-1H-pyrrol- | C₁₉H₂₂Cl₂N₆O₅S; | 30 |
| | 2-yl)carbonyl]amino}-3- | NMR: 1.87 (m, 1 H), 2.24 (s, 3 H), | |
| | (methoxyimino)piperidin-1- | 2.49 (m, 1 H), 2.71 (d, 2 H), | |
| | yl]-4-[(methylamino)carbonyl]- | 3.62 (m, 1 H), 3.71 (s, 3 H), 3.75 (m, 1 | |
| | 1,3-thiazole-5- | H), 3.92 (s, 3 H), 4.23 (d, 1 H), | |
| | carboxylate | 4.80-5.02 (m, 2 H), 7.75 (d, 1 H), | |
| | | 8.30 (m, 1 H), 12.28 (s, 1 H) | |
| 32 | 3,4-Dichloro-N-[(3E)-1-(5- | MS (ES) (M + H)⁺: 4432 for | 2-chloro-1,3- |
| | formyl-1,3-thiazol-2-yl)-3- | C₁₆H₁₇Cl₂N₅O₃S; | thiazole-5- |
| | (methoxyimino)piperidin-4- | NMR: 1.95 (m, 1 H), 2.24 (s, 3 H), | carbaldehyde |
| | yl]-5-methyl-1H-pyrrole-2- | 2.41 (m, 1 H), 3.51 (s, 3 H), 3.63 (m, | |
| | carboxamide | 1 H), 3.91 (s and m, 4 H), 4.22 (d, 1 | |
| | | H), 4.94 (m, 1 H), 5.16 (d, 1 H), | |
| | | 7.76 (d, 1 H), 8.23 (s, 1 H), 9.74 (s, 1 H), | |
| | | 12.10 (s, 1 H) | |
| 33 | Ethyl 2-[(3E)-4-{[(3,4- | MS (ES) (M + H)⁺: 575 for | Intermediate |
| | dichloro-5-methyl-1H-pyrrol- | C₂₂H₂₈Cl₂N₆O₆S; | 20 |
| | 2-yl)carbonyl]amino}-3- | NMR: 1.22 (s, 3 H), 1.91 (m, 1 H), | |
| | (methoxyimino)piperidin-1- | 2.29 (s, 3 H), 2.41 (m, 1 H), 3.31 (m, | |
| | yl]-4-{[(2-methoxyethyl)amino]carbonyl}- | 4 H), 3.58 (m, 1 H), 3.95 (s and m, 4 | |
| | 1,3- | H), 4.22 (m, 1 H), 4.94 (m, 2 H), | |
| | thiazole-5-carboxylate | 7.76 (d, 1 H), 8.57 (s, 1 H), 9.73 (s, | |
| | | 1 H), 12.13 (s, 1 H) | |
| 34 | Methyl 2-[(3E)-4-{[(3,4- | MS (ES) (M + H)⁺: 541 for | Intermediate |
| | dichloro-5-methyl-1H-pyrrol- | C₂₀H₂₂Cl₂N₈O₄S; NMR: 1.95 (m, 1 | 43 |
| | 2-yl)carbonyl]amino}-3- | H), 2.21 (s, 3 H), 3.63 (m, 1 H), | |
| | (methoxyimino)piperidin-1- | 3.71 (s, 6 H), 3.85 (s, 3 H), 4.22 (d, 1 H), | |
| | yl]-4-(1-methyl-1H-1,2,4- | 4.78-5.03 (m, 2 H), 7.71 (d, 1 H), | |
| | triazol-5-yl)-1,3-thiazole-5- | 8.01 (s, 1 H), 12.15 (s, 1 H) | |
| | carboxylate | | |
| 35 | Allyl 4-(aminocarbonyl)-2- | MS (ES) (M + H)⁺: 529 for | Intermediate |
| | [(3E)-4-{[(3,4-dichloro-5- | C₂₀H₂₂Cl₂N₆O₅S; NMR: 1.88 (m, 1 | 28 |
| | methyl-1H-pyrrol-2- | H), 2.21 (s, 3 H), 2.35 (m, 1 H), | |
| | yl)carbonyl]amino}-3- | 3.42 (m, 4 H), 3.61 (m, 1 H), 3.81 (m, 1 | |
| | (methoxyimino)piperidin-1- | H), 3.92 (s, 3 H), 4.15 (d, 1 H), | |
| | yl]-1,3-thiazole-5-carboxylate | 4.72 (d, 2 H), 4.91 (m, 1 H), 5.02 (d, 1 | |
| | | H), 5.22 (d, 1 H), 5.35 (d, 1 H), | |
| | | 5.93 (m, 1 H), 7.58-7.73 (m, 2 H), | |
| | | 7.91 (s, 1 H), 12.15 (s, 1 H) | |
| 36 | Methyl 2-[(3E)-4-{[(3,4- | MS (ES) (M + H)⁺: 584 for | Intermediate |
| | dichloro-5-methyl-1H-pyrrol- | C₂₃H₂₇Cl₂N₇O₅S; NMR: 1.88 (m, 1 | 44 |
| | 2-yl)carbonyl]amino}-3- | H), 2.21 (s, 3 H), 2.35 (m, 1 H), | |
| | (methoxyimino)piperidin-1- | 3.21 (s, 3 H), 3.33 (s, 3 H), 3.49 (t, 2 H), | |
| | yl]-4-[1-(2-methoxyethyl)- | 3.58 (m, 4 H), 3.69 (m, 1 H), 3.85 (s, | |
| | 1H-imidazol-2-yl]-1,3- | 3 H), 4.03 (t, 2 H), 4.15 (d, 1 H), | |
| | thiazole-5-carboxylate | 4.91 (m, 1 H), 5.03 (d, 1 H), 7.02 (s, | |
| | | 1 H), 7.33 (s, 1 H), 7.74 (d, 2 H), | |
| | | 12.15 (s, 1 H). | |
| 37 | Methyl 2-[(3E)-4-{[(3,4- | MS (ES) (M + H)⁺: 570 for | Intermediate |
| | dichloro-5-methyl-1H-pyrrol- | C₂₂H₂₅Cl₂N₇O₅S | 45 |
| | 2-yl)carbonyl]amino}-3- | | |
| | (methoxyimino)piperidin-1- | | |
| | yl]-4-[1-(methoxymethyl)- | | |
| | 1H-imidazol-2-yl]-1,3- | | |
| | thiazole-5-carboxylate | | |

### Example 38

### cis(±)Methyl 2-[4-{[(3,4-dichloro-5-methyl-1H-pyrrol-2-yl)carbonyl]amino}-3-(1H-imidazol-1-yl)piperidin-1-yl]-1,3-thiazole-5-carboxylate

A solution of cis(±)-3,4-dichloro-N-[3-(1H-imidazol-1-yl)piperidin-4-yl]-5-methyl-1H-pyrrole-2-carboxamide dihydrochloride (Intermediate 67, 0.40 g, 0.96 mmol), methyl 2-bromo-1,3-thiazole-5-carboxylate (0.214 g, 0.96 mmol), DIEA (0.48 ml, 2.9 mmol) and 1-methyl-2-pyrrolidinone (3 ml) were heated to 85° C. in a microwave reactor for 45 minutes. The resultant solution was cooled to room temperature and concentrated under reduced pressure. The crude residue was purified by flash chromatography (methanol/DCM gradient, 1-5%) to provide the title compound (0.270 g). MS (ESP) (M+H)⁺: 483 for C₁₉H₂₀Cl₂N₆O₃S; NMR: 1.98 (m, 2H), 2.17 (s, 3H), 3.60 (m, 1H), 3.76 (s, 3H), 3.96 (m, 2H), 4.21 (m, 1H), 4.57 (m, 1H), 4.84 (m, 1H), 6.89 (s, 1H), 6.96 (d, 1H), 7.12 (s, 1H), 7.58 (s, 1H), 7.90 (s, 1H), 12.03 (s, 1H).

### Examples 39-40

The following examples were prepared using the general method described for Example 38 from methyl 2-bromo-1,3-thiazole-5-carboxylate the starting material (SM) indicated.

| Ex | Compound | Data | SM |
|---|---|---|---|
| 39 | cis(±)Methyl 2-[4-{[(3,4- | MS (ESP) (M + H)⁺: 484 for | Intermediate |
| | dichloro-5-methyl-1H- | C₁₈H₁₉C₁₂N₇O₃S | 68 |
| | pyrrol-2-yl)carbonyl]amino}- | NMR: 1.93 (m, 1 H), 2.16 (s, 3 H), | |
| | 3-(1H-1,2,4- | 2.18 (m, 1 H), 3.58 (m, 1 H), 3.74 (s, 3 H), | |
| | triazol-1-yl)piperidin-1- | 3.98 (m, 2 H), 4.34 (m, 1 H), 4.70 (m, 1 | |
| | yl]-1,3-thiazole-5- | H), 5.03 (m, 1 H), 7.20 (d, 1 H), 7.82 (s, | |
| | carboxylate | 1 H), 7.97 (s, 1 H), 8.50 (s, 1 H), | |
| | | 12.06 (s, 1 H) | |
| 40 | cis(±)Methyl 2-(3-(3- | MS (ES) (M + H)⁺: 518 for | Intermediate |
| | chloro-1H-1,2,4-triazol-1- | C₁₈H₁₈Cl₃N₇O₃S | 69 |
| | yl)-4-{[(3,4-dichloro-5- | NMR: 1.94 (m, 1 H), 2.16 (m, 3 H), | |
| | methyl-1H-pyrrol-2-yl)carbonyl]amino}piperidin- | 3.32 (s, 2 H), 3.56 (t, 1 H), 3.73 (s, 3 H), | |
| | 1-yl)-1,3-thiazole-5- | 3.96 (m, 1 H), 4.33 (dd, 1 H), 4.66 (m, 1 H), | |
| | carboxylate | 4.97 (m, 1 H), 7.27 (d, 1 H), 7.82 (s, 1 | |
| | | H), 8.53 (s, 1 H), 12.03 (s, 1 H) | |

### Example 41

### cis(±)Ethyl 2-[4-{[(3,4-dichloro-5-methyl-1H-pyrrol-2-yl)carbonyl]amino}-3-(1H-imidazol-1-yl)piperidin-1-yl]-1,3-benzothiazole-7-carboxy late

A solution of cis(±)-3,4-dichloro-N-[3-(1H-imidazol-1-yl)piperidin-4-yl]-5-methyl-1H-pyrrole-2-carboxamide dihydrochloride (Intermediate 67, 0.25 g, 0.60 mmol), ethyl 2-bromo-1,3-benzothiazole-7-carboxylate (prepared as described in WO2006/087543, 0.17 g, 0.60 mmol), DIEA (0.30 ml, 1.8 mmol) and 1-methyl-2-pyrrolidinone (2 ml) were heated to 85° C. in a microwave reactor for 45 minutes. The resultant solution was cooled to room temperature and concentrated under reduced pressure. The crude residue was purified by flash chromatography (methanol/DCM gradient, 1-10%) to provide the title compound (0.120 g). MS (ESP) (M+H)⁺: 547 for C₂₄H₂₄Cl₂N₆O₃S; NMR: 1.36 (t, 3H), 2.00 (m, 2H), 2.18 (s, 3H), 3.65 (m, 1H), 3.98-4.20 (m, 2H), 4.30-4.43 (m, 3H), 4.59 (m, 1H), 4.88 (m, 1H), 6.88 (s, 1H), 6.96 (d, 1H), 7.17 (s, 1H), 7.46 (t, 1H), 7.61 (s, 1H), 7.75 (m, 2H), 12.04 (s, 1H).

### Examples 42-45

The following examples were prepared by sodium hydroxide hydrolysis as described in WO2006087543using the starting material (SM) indicated.

| Ex | Compound | Data | SM |
|---|---|---|---|
| 42 | cis(±)2-[4-{[(3,4-Dichloro- | MS (ESP) (M + H)⁺: 469 for C₁₈H₁₈Cl₂N₆O₃S | Example |
| | 5-methyl-1H-pyrrol-2- | NMR: 1.96 (m, 2 H), 2.18 (s, 3 H), 3.60 (m, | 38 |
| | yl)carbonyl]amino}-3-(1H- | 1 H), 3.98 (m, 2 H), 4.26 (m, 1 H), 4.61 (m, | |
| | imidazol-1-yl)piperidin-1- | 1 H), 4.88 (m, 1 H), 7.13 (s, 1 H), 7.20 (d, 1 | |
| | yl]-1,3-thiazole-5- | H), 7.30 (s, 1 H), 7.81 (s, 1 H), 8.05 (s, 1 H), | |
| | carboxylic acid | 12.13 (s, 1 H), 12.90 (br s, 1 H) | |
| 43 | cis(±)2-[4-{[(3,4-Dichloro- | MS (ESP) (M + H)⁺: 470 for C₁₇H₁₇Cl₂N₇O₃S | Example |
| | 5-methyl-1H-pyrrol-2- | NMR: 1.94 (m, 1 H), 2.16 (s, 3 H), 2.18 (m, | 39 |
| | yl)carbonyl]amino}-3-(1H- | 1 H), 3.56 (m, 1 H), 3.98 (m, 2 H), 4.35 (m, | |
| | 1,2,4-triazol-1- | 1 H), 4.69 (m, 1 H), 5.01 (m, 1 H), 7.25 (d, 1 | |
| | yl)piperidin-1-yl]-1,3- | H), 7.73 (s, 1 H), 7.98 (s, 1 H), 8.50 (s, 1 H), | |
| | thiazole-5-carboxylic acid | 12.06 (s, 1 H), 12.69 (br s, 1 H) | |
| 44 | cis(±)2-[4-{[(3,4-Dichloro- | MS (ESP) (M + H)⁺: 519 for C₂₂H₂₀Cl₂N₆O₃S | Example |
| | 5-methyl-1H-pyrrol-2- | NMR: 2.02 (m, 2 H), 2.19 (s, 3 H), 3.73 (m, | 41 |
| | yl)carbonyl]amino}-3-(1H- | 1 H), 4.09 (m, 2 H), 4.38 (m, 1 H), 4.66 (m, | |
| | imidazol-1-yl)piperidin-1- | 1 H), 4.94 (m, 1 H), 7.23 (s, 1 H), 7.37 (d, 1 | |
| | yl]-1,3-benzothiazole-7- | H), 7.44 (m, 2 H), 7.72 (m, 2 H), 8.30 (s, 1 | |
| | carboxylic acid | H), 12.21 (s, 1 H), 13.60 (br s, 1 H) | |
| 45 | cis(±)2-(3-(3-Chloro-1H- | MS (ES) (M + H)⁺: 504 for C₁₇H₁₆Cl₃N₇O₃S | Example |
| | 1,2,4-triazol-1-yl)-4-{[(3,4- | NMR: 1.92 (m, 1 H), 2.16 (m, 4 H), 3.54 (m, | 40 |
| | dichloro-5-methyl-1H- | 2 H), 3.92 (m, 2 H), 4.33 (dd, 1 H), 4.66 (m, | |
| | pyrrol-2-yl)carbonyl]amino}piperidin- | 1 H), 4.96 (m, 1 H), 7.31 (d, 1 H), 7.73 (s, 1 | |
| | 1-yl)-1,3- | H), 8.54 (s, 1 H), 12.06 (s, 1 H) | |
| | thiazole-5-carboxylic acid | | |

### Example 46

The following compound was prepared by HATU (1 eq) and methoxylamine hydrochloride (1 eq) in DMF and the starting material (SM) indicated by the procedure as described in WO2006/087543.

| Ex | Compound | Data | SM |
|---|---|---|---|
| 46 | cis(±)2-[4-{[(3,4-Dichloro-5- | MS (ESP) (M + H)⁺: 548 for | Example |
| | methyl-1H-pyrrol-2- | C₂₃H₂₃Cl₂N₇O₃S | 44 |
| | yl)carbonyl]amino}-3-(1H- | NMR: 1.98 (m, 2 H), 2.12 (s, 3 H), | |
| | imidazol-1-yl)piperidin-1-yl]- | 3.67 (s, 3 H), 3.72 (m, 1 H), 4.09 (m, 2 H), | |
| | N-methoxy-1,3- | 4.27 (m, 1 H), 4.65 (m, 1 H), 4.98 (m, 1 | |
| | benzothiazole-7-carboxamide | H), 7.34 (t, 1 H), 7.42 (d, 1 H), 7.50 (d, 1 | |
| | | H), 7.57-7.63 (m, 3 H), 9.06 (s, 1 H), | |
| | | 11.94 (s, 1 H), 12.05 (s, 1 H) | |

### Examples 47-48

The following compounds were prepared by HATU (1 eq) in DMF with the starting material(s) (SM) indicated by the procedure as described in WO2006/087543.

| Ex | Compound | Data | SM |
|---|---|---|---|
| 47 | cis(±)2-[4-{[(3,4- | MS (ESP) (M + H)⁺: 468 for | Example 42 |
| | Dichloro-5-methyl-1H- | C₁₈H₁₉Cl₂N₇O₂S | and |
| | pyrrol-2- | NMR: 1.93 (m, 2 H), 2.11 (s, 3 H), | ammonia in |
| | yl)carbonyl]amino}-3- | 3.54 (m, 1 H), 3.94 (m, 2 H), 4.15 (m, 1 H), | MeOH (3 |
| | (1H-imidazol-1- | 4.60 (m, 1 H), 4.92 (m, 1 H), 7.18 (br. s, 1 | eq) |
| | yl)piperidin-1-yl]-1,3- | H), 7.38 (d, 1 H), 7.59-7.75 (m, 3 H), | |
| | thiazole-5-carboxamide | 7.78 (s, 1 H), 9.03 (s, 1 H), 11.93 (s, 1 H) | |
| 48 | cis(±)Ethyl 2-[4-{[(3,4- | MS (ESP) (M + H)⁺: 613 for | Example 49 |
| | dichloro-5-methyl-1H- | C₂₄H₃₀Cl₂N₈O₅S | and (S)-(+)- |
| | pyrrol-2-yl)carbonyl]amino}- | NMR: 1.10 (d, 3 H), 1.21 (t, 3 H), | methoxy-2- |
| | 3-(1H-1,2,4- | 1.95 (m, 1 H), 2.17 (s, 3 H), 2.08-2.20 (m, 1 | propylamine |
| | triazol-1-yl)piperidin-1- | H), 3.18 (m, 1 H), 3.26 (s, 3 H), | |
| | yl]-4-({[(1S)-2-methoxy- | 3.30-3.45 (m, 2 H), 3.95-4.08 (m, 3 H), 4.16 (q, 2 | |
| | 1-methylethyl]amino}carbonyl)- | H), 4.26 (m, 1 H), 4.68 (m, 1 H), 5.01 (m, | |
| | 1,3-thiazole-5- | 1 H), 7.22 (d, 1 H), 8.02 (s, 1 H), 8.30 (d, | |
| | carboxylate | 1 H), 8.49 (s, 1 H), 12.05 (s, 1 H) | |

### Example 49

### cis(±)2-[4-{[(3,4-Dichloro-5-methyl-1H-pyrrol-2-yl)carbonyl]amino}-3-(1H-1,2,4-triazol-1-yl)piperidin-1-yl]-5-(ethoxycarbonyl)-1,3-thiazole-4-carboxylic acid

A solution of 2-chloro-5-(ethoxycarbonyl)-1,3-thiazole-4-carboxylic acid (WO2006087543, 0.235 g, 1 mmol), cis(±)3,4-dichloro-5-methyl-N-[3-(1H-1,2,4-triazol-1-yl)piperidin-4-yl]-1H-pyrrole-2-carboxamide dihydrochloride (Intermediate 68, 0.416 g, 1 mmol), DIEA (0.5 ml, 3 mmol) and 1-methyl-2-pyrrolidinone (3 ml) were heated to 70° C. with stirring for 3 h. The resultant solution was cooled to room temperature and concentrated under reduced pressure. The crude residue was purified by reversed-phase flash chromatography (water/acetonitrile gradient, 5-95% gradient) to provide the title compound (0.140 g). MS (ESP) (M−H)⁻: 540 for C₂₀H₂₁Cl₂N₇O₅S.

### Example 50

### cis(±)2-[4-{[(3,4-Dichloro-5-methyl-1H-pyrrol-2-yl)carbonyl]amino}-3-(1H-1,2,4-triazol-1-yl)piperidin-1-yl]-4-({[(1S)-2-methoxy-1-methylethyl]amino}carbonyl)-1,3-thiazole-5-carboxylic acid

A mixture of 1 eq of cis(±)ethyl 2-[4-{[(3,4-dichloro-5-methyl-1H-pyrrol-2-yl)carbonyl]amino}-3-(1H-1,2,4-triazol-1-yl)piperidin-1-yl]-4-({[(1S)-2-methoxy-1-methylethyl]amino}carbonyl)-1,3-thiazole-5-carboxylate (Example 48, 73 mg, 0.12 mmol) and barium hydroxide (61 mg, 0.36 mmol, 3 eq) in 3 ml MeOH and 2 ml water was stirred at room temperature for 8 h. The mixture was acidified to about pH 5 with 1N HCl and extracted 4 times with EtOAc. The EtOAc was concentrated and the residue was purified by reverse phase HPLC (20-40% CH₃CN gradient in water with 0.1% TFA) to afford 64 mg of product as a white solid. MS (ESP) (M+H)⁺: 585 for C₂₂H₂₆Cl₂N₈O₅S; NMR: 1.11 (d, 3H), 1.84 (m, 1 H), 2.10 (s, 3H), 2.08-2.15 (m, 1H), 3.19 (s, 3H), 3.20-3.47 (m, 3H), 3.93-4.20 (m, 3H), 4.39 (m, 1H), 4.63 (m, 1H), 4.98 (m, 1H), 7.20 (m, 1H), 7.94 (s, 1H), 8.49 (s, 1H), 8.87 (d, 1H), 12.00 (s, 1H), 16.30 (s, 1H).

### Example 51

### 2-((3S,6s,11R)-11-{[(3,4-Dichloro-5-methyl-1H-pyrrol-2-yl)carbonyl]amino}-3-ethoxy-1,5-dioxa-8-azaspiro[5.5]undec-8-yl)-1,3-thiazole-5-carboxylic acid and 2-((3R,6r,11R)-11-{[(3,4-Dichloro-5-methyl-1H-pyrrol-2-yl)carbonyl]amino}-3-ethoxy-1,5-dioxa-8-azaspiro[5.5]undec-8-yl)-1,3-thiazole-5-carboxylic acid (1:1)

Lithium hydroxide (0.19 mL, 0.39 mmol) was added to a suspension of methyl 2-((3R,6r,11R)-11-{[(3,4-dichloro-5-methyl-1H-pyrrol-2-yl)carbonyl]amino}-3-ethoxy-1,5-dioxa-8-azaspiro[5.5]undec-8-yl)-1,3-thiazole-5-carboxylate and methyl 2-43S,6s,11R)-11-{[(3,4-dichloro-5-methyl-1H-pyrrol-2-yl)carbonyl]amino}-3-ethoxy-1,5-dioxa-8-azaspiro[5.5]undec-8-yl)-1,3-thiazole-5-carboxylate (1:1) (0.068 g, 0.13 mmol, Example 58) in methanol (3 mL). After heating in the microwave at 100° C. for 30 minutes, the reaction mixture was cooled to room temperature and diluted with water. After acidifying with 1N HCl, the resulting precipitate was filtered, washed with water, and dried overnight to yield a white solid (0.053 g) MS (ES) (M+H)⁺: 519 for C₂₀H₂₄Cl₂N₄O₆S; NMR: 1.06-1.17 (m, 3H), 1.70 (s, 1H), 1.94 (s, 2H), 2.18 (s, 3H), 3.49 (d, J=4.90 Hz, 6H), 4.07 (s, 3H), 4.41 (s, 2H), 7.74 (s, 1H), 12.18 (s, 1H), 12.65 (s, 1H).

### Examples 52-57

The following examples were prepared by the procedure described in Example 51 from the starting materials (SM) indicated.

| Ex | Compound | Data | SM |
|---|---|---|---|
| 52 | 2-((rel-3R,6r,11R)-11- | MS (ES) (M + H)⁺: 505 for C₁₉H₂₂Cl₂N₄O₆S | Example |
| | {[(3,4-Dichloro-5-methyl- | NMR: 1.69 (s, 1 H), 1.89 (s, 1 H), | 59 |
| | 1H-pyrrol-2-yl)carbonyl]amino}- | 2.15-2.22 (m, 3 H), 3.14-3.26 (m, 1 H), | |
| | 3-methoxy-1,5- | 3.27-3.31 (m, 4 H), 3.51 (s, 1 H), 3.63-3.78 (m, | |
| | dioxa-8-azaspiro[5.5]undec- | 3 H), 4.06 (s, 2 H), 4.24-4.36 (m, 1 H), | |
| | 8-yl)-1,3-thiazole-5- | 4.94 (s, 1 H), 7.11 (d, 1 H), 7.77 (s, 1 H), | |
| | carboxylic acid | 12.17 (s, 1 H), 12.66 (s, 1 H) | |
| 53 | 2-((rel-3S,6s,11R)-11- | MS (ES) (M + H)⁺: 505 for C₁₉H₂₂Cl₂N₄O₆S | Example |
| | {[(3,4-Dichloro-5-methyl- | NMR: 1.66 (s, 1 H), 1.98 (s, 1 H), 2.18 (s, 3 | 60 |
| | 1H-pyrrol-2-yl)carbonyl]amino}- | H), 3.09-3.20 (m, 2 H), 3.23 (s, 3 H), | |
| | 3-methoxy-1,5- | 3.47 (s, 1 H), 3.72 (s, 1 H), 3.88 (s, 1 H), 3.99 (s, | |
| | dioxa-8-azaspiro[5.5]undec- | 1 H), 4.06-4.20 (m, 2 H), 5.07 (s, 1 H), | |
| | 8-yl)-1,3-thiazole-5- | 7.26 (d, 1 H), 7.76 (s, 1 H), 12.11 (s, 1 H), | |
| | carboxylic acid | 12.65 (s, 1 H) | |
| 54 | 2-[(3R,6r,11R)-11-{[(3,4- | MS (ES) (M + H)⁺: 505 for C₁₉H₂₂Cl₂N₄O₆S | Example |
| | Dichloro-5-methyl-1H- | NMR: 1.68 (s, 1 H), 1.88 (s, 1 H), 2.19 (s, 3 | 61 |
| | pyrrol-2-yl)carbonyl]amino}- | H), 3.08-3.20 (m, 1 H), 3.47 (s, 1 H), | |
| | 3-(hydroxymethyl)- | 3.60 (s, 1 H), 3.76 (s, 4 H), 3.87 (s, 1 H), 4.24 (d, | |
| | 1,5-dioxa-8-azaspiro[5.5]undec- | 2 H), 4.64 (s, 1 H), 5.01 (s, 1 H), 5.15 (s, 1 | |
| | 8-yl]-1,3-thiazole-5- | H), 7.04-7.18 (m, 1 H), 7.76 (d, 1 H), | |
| | carboxylic acid and 2- | 12.16 (s, 1 H), 12.66 (s, 1 H) | |
| | [(3S,6s,11R)-11-{[(3,4- | | |
| | dichloro-5-methyl-1H- | | |
| | pyrrol-2-yl)carbonyl]amino}- | | |
| | 3-(hydroxymethyl)- | | |
| | 1,5-dioxa-8-azaspiro[5.5]undec- | | |
| | 8-yl]-1,3-thiazole-5- | | |
| | carboxylic acid (1:1) | | |
| 55 | 2-(11-{[(3,4-Dichloro-5- | MS (ES) (M + H)⁺: 501 for C₂₀H₂₂Cl₂N₄O₅S | Example |
| | methyl-1H-pyrrol-2- | NMR: 0.36 (s, 2 H), 0.54 (s, 2 H), 1.75 (s, 1 | 62 |
| | yl)carbonyl]amino}-5,12- | H), 1.96 (s, 1 H), 2.19 (s, 3 H), 3.15 (s, 2 H), | |
| | dioxa-8-azadispiro[2.2.5.2]tridec- | 3.50 (s, 1 H), 3.73 (s, 1 H), 4.18 (s, 2 H), | |
| | 8-yl)-1,3-thiazole-5- | 4.39 (s, 2 H), 5.19 (s, 1 H), 7.30 (s, 1 H), | |
| | carboxylic acid | 7.77 (s, 1 H), 12.17 (s, 1 H) | |
| 56 | 2-(11-{[(3,4-Dichloro-5- | MS (ES) (M + H)⁺: 487 for C₁₉H₂₀Cl₂N₄O₅S | Example |
| | methyl-1H-pyrrol-2- | NMR: 1.71 (d, 1 H), 1.88 (s, 1 H), 2.18 (s, 3 | 63 |
| | yl)carbonyl]amino}-3- | H), 3.52 (s, 1 H), 3.73 (s, 1 H), 4.27 (s, 2 H), | |
| | methylene-1,5-dioxa-8- | 4.60 (s, 2 H), 4.96 (s, 4 H), 7.15 (d, 1 H), | |
| | azaspiro[5.5]undec-8-yl)- | 7.75-7.87 (m, 1 H), 12.17 (s, 1 H), 12.68 (s, | |
| | 1,3-thiazole-5-carboxylic | 1 H) | |
| | acid | | |
| 57 | 2-(11-{[(3,4-Dichloro-5- | MS (ES) (M + H)⁺: 503 for C₂₀H₂₄Cl₂N₄O₅S | Example |
| | methyl-1H-pyrrol-2- | NMR: 0.75 (s, 3 H), 1.06 (s, 3 H), 1.72 (s, 1 | 64 |
| | yl)carbonyl]amino}-3,3- | H), 1.84 (s, 1 H), 2.18 (s, 3 H), 3.11 (d, 1 H), | |
| | dimethyl-1,5-dioxa-8- | 3.45 (s, 3 H), 3.81 (t, 3 H), 4.32 (s, 1 H), | |
| | azaspiro[5.5]undec-8-yl)- | 5.07 (s, 1 H), 7.12 (d, 1 H), 7.76 (s, 1 H), | |
| | 1,3-thiazole-5-carboxylic | 12.17 (s, 1 H), 12.67 (s, 1 H) | |
| | acid | | |

### Example 58

### Methyl 2-((3R,6r,11R)-11-{[(3,4-dichloro-5-methyl-1H-pyrrol-2-yl)carbonyl]amino}-3-ethoxy-1,5-dioxa-8-azaspiro[5.5]undec-8-yl)-1,3-thiazole-5-carboxylate and Methyl 2-((3S,6s,11R)-11-{[(3,4-dichloro-5-methyl-1H-pyrrol-2-yl)carbonyl]amino}-3-ethoxy-1,5-dioxa-8-azaspiro[5.5]undec-8-0)-1,3-thiazole-5-carboxylate (1:1)

To a suspension of methyl 2-(4-{[(3,4-dichloro-5-methyl-1H-pyrrol-2-yl)carbonyl]amino}-3,3-dimethoxypiperidin-1-yl)-1,3-thiazole-5-carboxylate (0.15 g, 0.31 mmol, Example 10) in toluene was added 2-ethoxypropane-1,3-diol (0.3 mL) and p-toluenesulfonic acid (0.01 g). After heating at reflux overnight, the reaction mixture was cooled to room temperature. The crude reaction mixture was washed with sat. sodium bicarbonate (×2), dried with MgSO₄ and concentrated to an orange oil. Product was purified by silica gel flash column (gradient elution 25-100% EtOAc/DCM). Pure fractions were combined to give product as a yellow solid which is a mixture of diastereomers (0.068 g). MS (ES) (M+H)⁺: 533 for C₂₁H₂₆Cl₂N₄O₆S.

### Examples 59-64

The following Examples were prepared by the procedure described in Example 58 from the starting materials (SM) indicated.

| Ex | Compound | Data | SM |
|---|---|---|---|
| 59¹ | (diastereomer 1)Methyl 2- | MS (ES) (M + H)⁺: 519 for | Example 10 and |
| | ((rel-3R,6r,11R)-11- | C₂₀H₂₄Cl₂N₄O₆S | 2- |
| | {[(3,4-dichloro-5-methyl- | NMR: 1.69 (s, 1 H), 1.89 (s, 1 H), | methoxypropane- |
| | 1H-pyrrol-2-yl)carbonyl]amino}- | 2.15-2.22 (m, 3 H), 3.14-3.26 (m, 1 H), | 1,3-diol |
| | 3-methoxy-1,5- | 3.27-3.31 (m, 4 H), 3.51 (s, 1 H), | |
| | dioxa-8-azaspiro[5.5]undec- | 3.63-3.78 (m, 3 H), 4.06 (s, 2 H), | |
| | 8-yl)-1,3-thiazole- | 4.24-4.36 (m, 1 H), 4.94 (s, 1 H), 7.11 (d, 1 | |
| | 5-carboxylate | H), 7.77 (s, 1 H), 12.17 (s, 1 H), | |
| | | 12.66 (s, 1 H) | |
| 60¹ | (diastereomer 2)Methyl 2- | MS (ES) (M + H)⁺: 519 for | Example 10 and |
| | ((rel-3S,6s,11R)-11-{[(3,4- | C₂₀H₂₄Cl₂N₄O₆S | 2- |
| | dichloro-5-methyl-1H- | NMR: 1.64 (s, 1 H), 1.99 (s, 1 H), | methoxypropane- |
| | pyrrol-2-yl)carbonyl]amino}- | 2.18 (s, 3 H), 3.17 (d, 3 H), 3.23 (s, 3 H), | 1,3-diol |
| | 3-methoxy-1,5- | 3.72-3.77 (m, 2 H), 3.88 (s, 2 H), | |
| | dioxa-8-azaspiro[5.5]undec- | 3.93-4.04 (m, 1 H), 4.12 (d, 3 H), 5.06 (s, | |
| | 8-yl)-1,3-thiazole- | 1 H), 7.26 (d, 1 H), 7.85 (s, 1 H), | |
| | 5-carboxylate | 12.12 (s, 1 H) | |
| 61 | Methyl 2-[(3R,6r,11R)-11- | MS (ES) (M + H)⁺: 519 for | Example 10 and |
| | {[(3,4-dichloro-5-methyl- | C₂₀H₂₄Cl₂N₄O₆S | 2-(hydroxymethyl)propane- |
| | 1H-pyrrol-2-yl)carbonyl]amino}- | NMR: 1.67 (d, 1 H), 1.87 (s, 1 H), | 1,3-diol |
| | 3- | 2.19 (s, 3 H), 3.10-3.22 (m, 1 H), 3.27 (d, | |
| | (hydroxymethyl)-1,5- | 1 H), 3.49 (s, 2 H), 3.53-3.67 (m, 2 | |
| | dioxa-8-azaspiro[5.5]undec- | H), 3.75 (s, 4 H), 3.77-3.89 (m, 3 H), | |
| | 8-yl]-1,3-thiazole- | 4.22 (s, 2 H), 7.03-7.18 (m, 1 H), | |
| | 5-carboxylate and methyl | 7.82-7.87 (m, 1 H), 12.17 (d, 1 H) | |
| | 2-[(3S,6s,11R)-11-{[(3,4- | | |
| | dichloro-5-methyl-1H- | | |
| | pyrrol-2-yl)carbonyl]amino}- | | |
| | 3- | | |
| | (hydroxymethyl)-1,5- | | |
| | dioxa-8-azaspiro[5.5]undec- | | |
| | 8-yl]-1,3-thiazole- | | |
| | 5-carboxylate (1:1) | | |
| 62 | Methyl 2-(11-{[(3,4- | MS (ES) (M + H)⁺: 515 for | Example 10 and |
| | dichloro-5-methyl-1H- | C₂₁H₂₄Cl₂N₄O₅S | cyclopropane- |
| | pyrrol-2-yl)carbonyl]amino}- | | 1,1- |
| | 5,12-dioxa-8- | | diyldimethanol |
| | azadispiro[2.2.5.2]tridec- | | |
| | 8-yl)-1,3-thiazole-5- | | |
| | carboxylate | | |
| 63 | Methyl 2-(11-{[(3,4- | MS (ES) (M + H)⁺: 501 for | Example 10 and |
| | dichloro-5-methyl-1H- | C₂₀H₂₂Cl₂N₄O₅S | 2- |
| | pyrrol-2-yl)carbonyl]amino}- | | methylenepropane- |
| | 3-methylene-1,5- | | 1,3-diol |
| | dioxa-8-azaspiro[5.5]undec- | | |
| | 8-yl)-1,3-thiazole- | | |
| | 5-carboxylate | | |
| 64 | Methyl 2-(11-{[(3,4- | MS (ES) (M + H)⁺: 517 for | Example 10 and |
| | dichloro-5-methyl-1H- | C₂₁H₂₆Cl₂N₄O₅S | 2,2- |
| | pyrrol-2-yl)carbonyl]amino}- | NMR: 0.75 (s, 6 H), 1.03-1.09 (m, 2 | dimethylpropane- |
| | 3,3-dimethyl-1,5- | H), 1.71 (s, 1 H), 1.85 (s, 1 H), 2.18 (s, | 1,3-diol |
| | dioxa-8-azaspiro[5.5]undec- | 3 H), 3.47 (s, 2 H), 3.75 (s, 3 H), | |
| | 8-yl)-1,3-thiazole- | 3.77-3.86 (m, 2 H), 4.33 (t, 2 H), 5.06 (s, 1 | |
| | 5-carboxylate | H), 7.86 (s, 1 H), 12.17 (s, 1 H) | |

¹In instances where diastereomers were separated, separation was accomplished by silica gel flash column chromatography (gradient elution 25-100% EtOAc/DCM). The first diastereomer recovered was assigned diastereomer 1 and the second compound recovered was assigned diastereomer 2.

### Example 65

### cis(±)Methyl 2-(3-azido-4-{[(3,4-dichloro-5-methyl-1H-pyrrol-2-yl)carbonyl]amino}piperidin-1-yl)-1,3-thiazole-5-carboxylate

cis(±)N-(3-Azidopiperidin-4-yl)-3,4-dichloro-5-methyl-1H-pyrrole-2-carboxamide hydrochloride (Intermediate 73, 724 mg, 22 mmol) was dissolved in DMF (10 ml) and DIEA (1.48 g; 1.9 ml; 11.5 mmol; 5 eq.). Methyl 2-bromo-1,3-thiazole-5-carboxylate (536 mg; 2.4 mmol; 1.05 eq.) was added and the reaction was heated to 80° C. The displacement was monitored by LC/MS. The reaction was diluted with EtOAc and washed with H₂O (×2), brine and dried over Na₂SO₄. The crude was purified by flash column chromatography (70% EtOAc/hexanes) to give 733 mg of the title compound in 69% yield. LC/MS (ES) (M+H)⁺: 458 for C₁₆H₁₇Cl₂N₇O₃S; NMR: 1.82 (m, 2H), 2.19 (s, 3H), 3.35 (m, 1H), 3.64 (d, 1H), 3.75 (s, 3H), 3.95 (d, 1H), 4.24 (m, 3H), 7.23 (d, 1H), 7.86 (s, 1H), 12.18 (s, 1H).

### Example 66

### cis(±)-2-(3-Azido-4-{[(3,4-dichloro-5-methyl-1H-pyrrol-2-yl)carbonyl]amino}piperidin-1-yl)-1,3-thiazole-5-carboxylic acid

cis(±)Methyl 2-(3-azido-4-{[(3,4-dichloro-5-methyl-1H-pyrrol-2-yl)carbonyl]amino}piperidin-1-yl)-1,3-thiazole-5-carboxylate (Example 65, 100 mg) was saponified with 1 eq 1N NaOH according to the procedure described in WO2006/087543. LC/MS (ES) (M+H)⁺: 444 for C₁₅H₁₅Cl₂N₇O₃S; NMR: 1.82 (m, 2H), 2.21 (s, 3H), 3.38 (m, 1H), 3.64 (d, 1H), 3.92 (d, 1H), 4.24 (m, 3H), 7.23 (d, 1H), 7.79 (s, 1H), 12.14 (s, 1H), 12.71 (s, 1H).

### Example 67

### cis(±)Methyl 2-(3-amino-4-{[(3,4-dichloro-5-methyl-1H-pyrrol-2-yl)carbonyl]amino}piperidin-1-yl)-1,3-thiazole-5-carboxylate

cis(±)Methyl 2-(3-azido-4-{[(3,4-dichloro-5-methyl-1H-pyrrol-2-yl)carbonyl]amino}piperidin-1-yl)-1,3-thiazole-5-carboxylate (Example 65, 3 g; 6.5 mol) was dissolved in THF (40 ml) and H₂O (10 ml). Resin-bound triphenylphosphine (8.66 g; 13.1 mmol; 2 eq.) was added and the slurry was stirred at 80° C. for 12 hours. The resin was filtered off and the filtrate was concentrated to a solid. No further purification. LC/MS (ES⁺) (M+H)⁺: 432, 434 for C₁₆H₁₉Cl₂N₅O₃S.

### Example 68

### cis(±)2-(3-Amino-4-{[(3,4-dichloro-5-methyl-1H-pyrrol-2-yl)carbonyl]amino}piperidin-1-yl)-1,3-thiazole-5-carboxylic acid

cis(±)Methyl 2-(3-amino-4-{[(3,4-dichloro-5-methyl-1H-pyrrol-2-yl)carbonyl]amino}piperidin-1-yl)-1,3-thiazole-5-carboxylate (Example 67, 100 mg) was saponified with 1 eq 1N NaOH in accordance with the procedure described in WO2006/087543. LC/MS (ES) (M+H)⁺: 418 for C₁₅H₁₇Cl₂N₅O₃S.

### Example 69

### Methyl 2-((3S,4R)-3-azido-4-{[(3,4-dichloro-5-methyl-1H-pyrrol-2-yl)carbonyl]amino}piperidin-1-yl)-1,3-thiazole-5-carboxylate

N-[(3S,4R)-3-Azidopiperidin-4-yl]-3,4-dichloro-5-methyl-1H-pyrrole-2-carboxamide hydrobromide (Intermediate 92, 69 mg; 0.22 mmol) was dissolved in NMP (2 ml) and DIEA (84 mg; 1100; 0.65 mmol; 3 equiv.). Methyl 2-bromo-1,3-thiazole-5-carboxylate (53 mg; 0.24 mmol; 1.1 equiv.) was added in as single portion and the reaction was heated to 70° C. Monitored by LC/MS. Dilute the reaction with EtOAc, wash with water (×2) then brine. Dried the organic layer over Na₂SO₄, filtered ands concentrated. Purified by flash column chromatography. LC/MS (ES) (M+H)⁺: 458, 460 for C₁₆H₁₇Cl₂N₇O₃S.

### Examples 70-71

The following compounds were prepared in accordance to procedure described above for Example 69 with starting material listed.

| Ex | Compound | Data | SM |
|---|---|---|---|
| 70 | Methyl 2-[(3S,4R)-4-{[(3,4-dichloro-5- | LC/MS (ES⁺) (M + H)⁺: | Intermediate |
| | methyl-1H-pyrrol-2-yl)carbonyl]amino}-3- | 484 for | 91 |
| | (1H-1,2,3-triazol-1-yl)-1-piperidinyl]-1,3- | C₁₈H₁₉Cl₂N₇O₃S | |
| | thiazole-5-carboxylate | | |
| 71 | Methyl 2-[(3S,4R)-3-(4-chloro-1H-1,2,3- | LC/MS (ES⁺) (M + H)⁺: | Intermediate |
| | triazol-1-yl)-4-[(3,4-dichloro-5-methyl-1H- | 518 for | 94 |
| | pyrrole-2-carbonyl)amino]-1-piperidinyl]- | C₁₈H₁₈Cl₃N₇O₃S | |
| | 1,3-thiazole-5-carboxylate | | |

### Example 72

### 2-[(3S,4R)-4-{[(3,4-Dichloro-5-methyl-1H-pyrrol-2-yl)carbonyl]amino}-3-(1H-1,2,3-triazol-1-yl)piperidin-1-yl]-5-(ethoxycarbonyl)-1,3-thiazole-4-carboxylic acid

3,4-Dichloro-5-methyl-N-[(3S,4R)-3-(1H-1,2,3-triazol-1-yl)piperidin-4-yl]-1H-pyrrole-2-carboxamide hydrobromide (Intermediate 91, 0.74 mmol) was dissolved in NMP (3 ml) and DIEA (0.3 ml; 234 mg; 1.8 mmol; 2.5 equiv.). 2-Chloro-5-(ethoxycarbonyl)-1,3-thiazole-4-carboxylic acid (175 mg; 0.74 mmol) was added in as single portion and the reaction was heated to 70° C. Monitored by LC/MS. The reaction was acidified with 2N HCl and the product was precipitated from solution by the addition of water. The solids were filtered and dried under vacuum to yield 157 mg of the title compound. LC/MS (ES⁺) (M+H)⁺: 542, 544 for C₂₀H₂₁Cl₂N₇O₅S.

### Examples 73-81

The following examples were synthesized as described for Example 72 using the starting material listed.

| Ex | Compound | Data | SM |
|---|---|---|---|
| 73 | 2-[(3S,4R)-3-Azido-4-[(3,4-dichloro-5- | LC/MS (ES⁺) (M + H)⁺: | Intermediate |
| | methyl-1H-pyrrole-2-carbonyl)amino]-1- | 516, 518 for | 92 |
| | piperidinyl]-5-ethoxycarbonyl-1,3-thiazole-4- | C₁₈H₁₉Cl₂N₇O₅S | |
| | carboxylic acid | | |
| 74 | 2-[(3S,4R)-3-(4-Chloro-1H-1,2,3-triazol-1- | LC/MS (ES⁺) (M + H)⁺: | Intermediate |
| | yl)-4-[(3,4-dichloro-5-methyl-1H-pyrrole-2- | 576, 578 for | 94 |
| | carbonyl)amino]-1-piperidinyl]-5- | C₂₀H₂₀Cl₃N₇O₅S | |
| | ethoxycarbonyl-1,3-thiazole-4-carboxylic | | |
| | acid | | |
| 75 | 2-[(3S,4R)-4-[(3,4-Dichloro-5-methyl-1H- | LC/MS (ES⁺) (M + H)⁺: | Intermediate |
| | pyrrole-2-carbonyl)amino]-3-(4-methyl-1H- | 556, 558 for | 93 |
| | 1,2,3-triazol-1-yl)-1-piperidinyl]-5- | C₂₁H₂₃Cl₂N₇O₅S | |
| | ethoxycarbonyl-1,3-thiazole-4-carboxylic | | |
| | acid | | |
| 76 | 2-[(3S,4R)-3-(4-Bromo-1H-1,2,3-triazol-1- | LC/MS (ES⁺) (M + H)⁺: | Intermediate |
| | yl)-4-[(3,4-dichloro-5-methyl-1H-pyrrole-2- | 620, 622, 624 for | 95 |
| | carbonyl)amino]-1-piperidinyl]-5- | C₂₀H₂₀BrCl₂N₇O₅S | |
| | ethoxycarbonyl-1,3-thiazole-4-carboxylic | | |
| | acid | | |
| 77 | 2-[(3S,4R)-3-(4-Cyano-1H-1,2,3-triazol-1- | LC/MS (ES⁺) (M + H)⁺: | Intermediate |
| | yl)-4-[(3,4-dichloro-5-methyl-1H-pyrrole-2- | 567, 569 for | 99 |
| | carbonyl)amino]-1-piperidinyl]-5- | C₂₁H₂₀Cl₂N₈O₅S | |
| | ethoxycarbonyl-1,3-thiazole-4-carboxylic | | |
| | acid | | |
| 78 | 2-[(3S,4R)-4-[(3,4-Dichloro-5-methyl-1H- | LC/MS (ES⁺) (M + H)⁺: | Intermediate |
| | pyrrole-2-carbonyl)amino]-3-[4- | 572, 574 for | 96 |
| | (hydroxymethyl)-1H-1,2,3-triazol-1-yl]-1- | C₂₁H₂₃Cl₂N₇O₆S | |
| | piperidinyl]-5-ethoxycarbonyl-1,3-thiazole-4- | | |
| | carboxylic acid | | |
| 79 | 2-[(3S,4R)-4-[(3,4-Dichloro-5-methyl-1H- | LC/MS (ES⁺) (M + H)⁺: | Intermediate |
| | pyrrole-2-carbonyl)amino]-3-[4- | 586, 588 for | 98 |
| | (methoxymethyl)-1H-1,2,3-triazol-1-yl]-1- | C₂₂H₂₅Cl₂N₇O₆S | |
| | piperidinyl]-5-ethoxycarbonyl-1,3-thiazole-4- | | |
| | carboxylic acid | | |
| 80 | 2-[(3S,4R)-3-[4-(Cyanomethyl)-1H-1,2,3- | LC/MS (ES⁺) (M + H)⁺: | Intermediate |
| | triazol-1-yl]-4-[(3,4-dichloro-5-methyl-1H- | 581, 583 for | 100 |
| | pyrrole-2-carbonyl)amino]-1-piperidinyl]-5- | C₂₂H₂₂Cl₂N₈O₅S | |
| | ethoxycarbonyl-1,3-thiazole-4-carboxylic | | |
| | acid | | |
| 81 | 2-[(3S,4R)-4-[(3,4-Dichloro-5-methyl-1H- | LC/MS (ES⁺) (M + H)⁺: | Intermediate |
| | pyrrole-2-carbonyl)amino]-3-[4- | 574, 576 for | 97 |
| | (fluoromethyl)-1H-1,2,3-triazol-1-yl]-1- | C₂₁H₂₂Cl₂FN₇O₅S | |
| | piperidinyl]-5-ethoxycarbonyl-1,3-thiazole-4- | | |
| | carboxylic acid | | |

### Example 82

### 2-[(3S,4R)-4-{[(3,4-Dichloro-5-methyl-1H-pyrrol-2-yl)carbonyl]amino}-3-(1H-1,2,3-triazol-1-yl)piperidin-1-yl]-1,3-benzothiazole-7-carboxamide

2-[(3S,4R)-4-{[(3,4-Dichloro-5-methyl-1H-pyrrol-2-yl)carbonyl]amino}-3-(1H-1,2,3-triazol-1-yl)piperidin-1-yl]-1,3-benzothiazole-7-carboxylic acid (Example 124, 57 mg) was dissolved in anhydrous NMP (2 mL) under an argon atmosphere, followed by the addition of HATU (44 mg) and DIEA (30 μL) and the reaction was stirred at room temperature for 40 mins. Ammonia (2.0 M in methanol) (0.42 mL) was added, and the reaction was stirred overnight at room temperature. The reaction mixture was then added slowly via pipette to rapidly stirring dilute aqueous NH₄Cl (50 mL), cooled to 0° C., filtered and rinsed with deionized water. The crude product was purified by trituration with CH₃CN to yield the title compound as a beige solid (19 mg, 33.3%). MS (ES) (M+H)⁺: 519 for C₂₁H₂₀Cl₂N₈O₂S; NMR: 2.01 (m, 1H), 2.12 (m, 1H), 2.15 (s, 3H), 3.64 (m, 1H), 4.09 (dd, 1H), 4.13 (m, 1 H), 4.42 (dd, 1H), 4.75 (m, 1H), 5.25 (q, 1H), 7.13 (d, 1H), 7.35 (t, 1H), 7.56 (d, 1H), 7.65 (broad s, 1H), 7.69 (s, 1H), 7.71 (d, 1H), 8.11 (d, 1H), 8.23 (broad s, 1H), 12.02 (s, 1H).

### Example 83

### Ethyl 2-[(3S,4R)-4-{[(3,4-dichloro-5-methyl-1H-pyrrol-2-yl)carbonyl]amino}-3-(1H-1,2,3-triazol-1-yl)piperidin-1-yl]-4-{[2-methoxyethyl)amino]carbonyl}-1,3-thiazole-5-carboxylate

2-[(3S,4R)-4-{[(3,4-Dichloro-5-methyl-1H-pyrrol-2-yl)carbonyl]amino}-3-(1H-1,2,3-triazol-1-yl)piperidin-1-yl]-5-(ethoxycarbonyl)-1,3-thiazole-4-carboxylic acid (Example 72, 60 mg; 0.11 mmol) was added to a pre-mixed solution of HATU (65 mg; 0.17 mmol; 1.5 equiv.) and DIEA (0.1 ml; 78 mg; 0.6 mmol; 5.5 equiv.) in NMP (0.5 ml). The solution was stirred for 30 minutes. Added (2-methoxyethyl)amine (25 mg; 0.33 mmol) in a single portion. Monitored by LC/MS. Diluted with EtOAc and then washed with water. Dried the organic layer over Na₂SO₄, filtered and concentrated. LC/MS (ES) (M+H)⁺: 599, 601 for C₂₃H₂₈Cl₂N₈O₅S.

### Examples 84-99

The following examples were synthesized from Example 83 and the starting material described.

| Ex | Compound | Data | SM |
|---|---|---|---|
| 84 | Ethyl 2-[(3S,4R)-3-azido-4-[(3,4-dichloro-5- | LC/MS (ES⁺) (M + H)⁺: | Example |
| | methyl-1H-pyrrole-2-carbonyl)amino]-1- | 573, 575 for | 73 |
| | piperidinyl]-4-(2-methoxyethylcarbamoyl)-1,3- | C₂₁H₂₆Cl₂N₈O₅S | |
| | thiazole-5-carboxylate | | |
| 85 | Ethyl 2-[(3S,4R)-3-(4-chloro-1H-1,2,3-triazol- | LC/MS (ES⁺) (M + H)⁺: | Example |
| | 1-yl)-4-[(3,4-dichloro-5-methyl-1H-pyrrole-2- | 633, 635 for | 74 |
| | carbonyl)amino]-1-piperidinyl]-4-(2- | C₂₃H₂₇Cl₃N₈O₅S | |
| | methoxyethylcarbamoyl)-1,3-thiazole-5- | | |
| | carboxylate | | |
| 86 | Ethyl 2-[(3S,4R)-3-(4-chloro-1H-1,2,3-triazol- | LC/MS (ES⁺) (M + H)⁺: | Example |
| | 1-yl)-4-[(3,4-dichloro-5-methyl-1H-pyrrole-2- | 678, 680 for | 74 |
| | carbonyl)amino]-1-piperidinyl]-4-(1,3- | C₂₅H₃₁Cl₃N₈O₆S | |
| | dimethoxypropan-2-ylcarbamoyl)-1,3-thiazole- | | |
| | 5-carboxylate | | |
| 87 | Ethyl 2-[(3S,4R)-3-(4-chloro-1H-1,2,3-triazol- | LC/MS (ES⁺) (M + H)⁺: | Example |
| | 1-yl)-4-[(3,4-dichloro-5-methyl-1H-pyrrole-2- | 615, 617 for | 74 |
| | carbonyl)amino]-1-piperidinyl]-4- | C₂₃H₂₅Cl₃N₈O₄S | |
| | (cyclopropylcarbamoyl)-1,3-thiazole-5- | | |
| | carboxylate | | |
| 88 | Ethyl 2-[(3S,4R)-4-[(3,4-dichloro-5-methyl- | LC/MS (ES⁺) (M + H)⁺: | Example |
| | 1H-pyrrole-2-carbonyl)amino]-3-(4-methyl- | 613, 615 for | 75 |
| | 1H-1,2,3-triazol-1-yl)-1-piperidinyl]-4-(2- | C₂₄H₃₀Cl₂N₈O₅S | |
| | methoxyethylcarbamoyl)-1,3-thiazole-5- | | |
| | carboxylate | | |
| 89 | Ethyl 2-[(3S,4R)-4-[(3,4-dichloro-5-methyl- | LC/MS (ES⁺) (M + H)⁺: | Example |
| | 1H-pyrrole-2-carbonyl)amino]-3-(4-methyl- | 657, 659 for | 75 |
| | 1H-1,2,3-triazol-1-yl)-1-piperidinyl]-4-(1,3- | C₂₆H₃₄Cl₂N₈O₆S | |
| | dimethoxypropan-2-ylcarbamoyl)-1,3-thiazole- | | |
| | 5-carboxylate | | |
| 90 | Ethyl 2-[(3S,4R)-4-[(3,4-dichloro-5-methyl- | LC/MS (ES⁺) (M + H)⁺: | Example |
| | 1H-pyrrole-2-carbonyl)amino]-3-(4-methyl- | 701, 703 for | 75 |
| | 1H-1,2,3-triazol-1-yl)-1-piperidinyl]-[[1,3- | C₂₈H₃₈Cl₂N₈O₇S | |
| | dimethoxy-2-(methoxymethyl)propan-2- | | |
| | yl]carbamoyl]-1,3-thiazole-5-carboxylate | | |
| 91 | Ethyl 2-[(3S,4R)-3-(4-bromo-1H-1,2,3-triazol- | LC/MS (ES⁺) (M + H)⁺: | Example |
| | 1-yl)-4-[(3,4-dichloro-5-methyl-1H-pyrrole-2- | 721, 723, 725 for | 76 |
| | carbonyl)amino]-1-piperidinyl]-4-(1,3- | C₂₅H₃₁BrCl₂N₈O₆S | |
| | dimethoxypropan-2-ylcarbamoyl)-1,3-thiazole- | | |
| | 5-carboxylate | | |
| 92 | Ethyl 2-[(3S,4R)-3-(4-cyano-1H-1,2,3-triazol- | LC/MS (ES⁺) (M + H)⁺: | Example |
| | 1-yl)-4-[(3,4-dichloro-5-methyl-1H-pyrrole-2- | 624 for | 77 |
| | carbonyl)amino]-1-piperidinyl]-4-(2- | C₂₄H₂₇Cl₂N₉O₅S. ¹H | |
| | methoxyethylcarbamoyl)-1,3-thiazole-5- | NMR: 1.20 (t, 3 H), | |
| | carboxylate | 1.97 (m, 1 H), 2.12 (m, 1 H), | |
| | | 2.15 (s, 3 H), 3.43 (m, 4 | |
| | | H), 3.64 (m, 1 H), | |
| | | 4.01 (m, 2 H), 4.12 (q, 2 H), | |
| | | 4.39 (m, 1 H), 4.78 (m, | |
| | | 1 H), 5.34 (m, 1 H), | |
| | | 7.27 (d, 1 H), 8.44 (t, 1 H), | |
| | | 9.05 (s, 1 H), 11.93 (s, 1 | |
| | | H) | |
| 93 | Ethyl 2-[(3S,4R)-3-[4-(cyanomethyl)-1H-1,2,3- | LC/MS (ES⁺) (M + H)⁺: | Example |
| | triazol-1-yl]-4-[(3,4-dichloro-5-methyl-1H- | 682, 684 for | 80 |
| | pyrrole-2-carbonyl)amino]-1-piperidinyl]-4- | C₂₇H₃₃Cl₂N₉O₆S | |
| | (1,3-dimethoxypropan-2-ylcarbamoyl)-1,3- | | |
| | thiazole-5-carboxylate | | |
| 94 | Ethyl 2-[(3S,4R)-4-[(3,4-dichloro-5-methyl- | LC/MS (ES⁺) (M + H)⁺: | Example |
| | 1H-pyrrole-2-carbonyl)amino]-3-[4- | 629, 631 for | 78 |
| | (hydroxymethyl)-1H-1,2,3-triazol-1-yl]-1- | C₂₄H₃₀Cl₂N₈O₆S | |
| | piperidinyl]-4-(2-methoxyethylcarbamoyl)-1,3- | | |
| | thiazole-5-carboxylate | | |
| 95 | Ethyl 2-[(3S,4R)-4-[(3,4-dichloro-5-methyl- | LC/MS (ES⁺) (M + H)⁺: | Example |
| | 1H-pyrrole-2-carbonyl)amino]-3-[4- | 673, 675 for | 78 |
| | (hydroxymethyl)-1H-1,2,3-triazol-1-yl]-1- | C₂₆H₃₄Cl₂N₈O₇S | |
| | piperidinyl]-4-(1,3-dimethoxypropan-2- | | |
| | ylcarbamoyl)-1,3-thiazole-5-carboxylate | | |
| 96 | Ethyl 2-[(3S,4R)-4-[(3,4-dichloro-5-methyl- | LC/MS (ES⁺) (M + H)⁺: | Example |
| | 1H-pyrrole-2-carbonyl)amino]-3-[4- | 643, 645 for | 79 |
| | (methoxymethyl)-1H-1,2,3-triazol-1-yl]-1- | C₂₅H₃₂Cl₂N₈O₆S | |
| | piperidinyl]-4-(2-methoxyethylcarbamoyl)-1,3- | | |
| | thiazole-5-carboxylate | | |
| 97 | Ethyl 2-[(3S,4R)-4-[(3,4-dichloro-5-methyl- | LC/MS (ES⁺) (M + H)⁺: | Example |
| | 1H-pyrrole-2-carbonyl)amino]-3-[4- | 687, 689 for | 79 |
| | (methoxymethyl)-1H-1,2,3-triazol-1-yl]-1- | C₂₇H₃₆Cl₂N₈O₇S | |
| | piperidinyl]-4-(1,3-dimethoxypropan-2- | | |
| | ylcarbamoyl)-1,3-thiazole-5-carboxylate | | |
| 98 | Ethyl 2-[(3S,4R)-4-[(3,4-dichloro-5-methyl- | LC/MS (ES⁺) (M + H)⁺: | Example |
| | 1H-pyrrole-2-carbonyl)amino]-3-[4- | 675, 677 for | 81 |
| | (fluoromethyl)-1H-1,2,3-triazol-1-yl]-1- | C₂₆H₃₃Cl₂FN₈O₆S | |
| | piperidinyl]-4-(1,3-dimethoxypropan-2- | | |
| | ylcarbamoyl)-1,3-thiazole-5-carboxylate | | |
| 99 | Ethyl 2-[(3S,4R)-4-{[(3,4-dichloro-5-methyl- | LC/MS (ES⁺) (M − H)⁻: | Example |
| | 1H-pyrrol-2-yl)carbonyl]amino}-3-(1H-1,2,3- | 643, 645 for | 72 |
| | triazol-1-yl)piperidin-1-yl]-4-({[2-methoxy-1- | C₂₅H₃₂Cl₂N₈O₆S. | |
| | (methoxymethyl)ethyl]amino}carbonyl)-1,3- | | |
| | thiazole-5-carboxylate | | |

### Example 100

### 2-[(3S,4R)-4-{[3,4-Dichloro-5-methyl-1H-pyrrol-2-yl)carbonyl]amino}-3-(1H-1,2,3-triazol-1-yl)piperidin-1-yl]-4-{[(2-methoxyethyl)amino]carbonyl}-1,3-thiazole-5-carboxylic acid

Ethyl 2-[(3S,4R)-4-{[(3,4-dichloro-5-methyl-1H-pyrrol-2-yl)carbonyl]amino}-3-(1H-1,2,3-triazol-1-yl)piperidin-1-yl]-4-{[(2-methoxyethyl)amino]carbonyl}-1,3-thiazole-5-carboxylate (Example 83, 0.11 mmol) was dissolved in aqueous methanol. Ba(OH)₂ was added in a single portion. The reaction was stirred for 30 minutes and monitored by LC/MS. Acidified the reaction to pH=1 with 2N HCl. The product was precipitated from solution by the addition of water. The solids were collected and dried in vacuum to give 36 mg of the title compound. LC/MS (ES⁺) (M+H)⁺: 571 for C₂₁H₂₄Cl₂N₈O₅S; NMR: 2.00 (m, 1H), 2.12 (m, 1 H), 2.17 (s, 3H), 3.26 (s, 3H), 3.49 (m, 5H), 4.03 (m, 2H), 4.48 (m, 1H), 4.78 (m, 1H), 5.23 (m, 1H), 7.18 (d, 1H), 7.74 (s, 1H), 8.16 (s. 1H), 9.32 (m, 1H), 12.04 (s, 1H).

### Examples 101-118

The following compounds were prepared as described for Example 100 from the starting material described.

| Ex | Compound | Data | SM |
|---|---|---|---|
| 101 | 2-[(3S,4R)-4-{[(3,4-Dichloro-5- | LC/MS (ES⁺) (M + H)⁺: 470, 472 for | Example 70 |
| | methyl-1H-pyrrol-2- | C₁₇H₁₇Cl₂N₇O₃S | |
| | yl)carbonyl]amino}-3-(1H-1,2,3- | NMR: 1.92 (m, 1 H), 2.11 (m, 1 H), | |
| | triazol-1-yl)piperidin-1-yl]-1,3- | 2.16 (s, 3 H), 3.58 (m, 1 H), | |
| | thiazole-5-carboxylic acid | 4.06 (m, 2 H), 4.33 (m, 1 H), 4.74 (m, 1 | |
| | | H), 5.29 (m, 1 H), 7.12 (d, 1 H), | |
| | | 7.71 (s, 2 H), 8.16 (s, 1 H), | |
| | | 12.00 (s, 1 H), 12.74 (s, 1 H) | |
| 102 | 2-((3S,4R)-3-Azido-4-{[(3,4- | LC/MS (ES⁺) (M + H)⁺: 443 for | Example 69 |
| | dichloro-5-methyl-1H-pyrrol-2- | C₁₅H₁₅Cl₂N₇O₃S | |
| | yl)carbonyl]amino}piperidin-1- | NMR: 1.81 (m, 2 H), 2.23 (s, 3 H), | |
| | yl)-1,3-thiazole-5-carboxylic acid | 3.31 (m, 1 H), 3.52 (d, 1 H), | |
| | | 3.95 (d, 1 H), 4.24 (m, 3 H), 7.23 (d, 1 | |
| | | H), 7.74 (s, 2 H), 12.02 (s, 1 H), | |
| | | 12.74 (s, 1 H) | |
| 103 | 2-((3S,4R)-3-Azido-4-{[(3,4- | LC/MS (ES⁺) (M + H)⁺: 545, 547 for | Example 84 |
| | dichloro-5-methyl-1H-pyrrol-2- | C₁₉H₂₂Cl₂N₈O₅S | |
| | yl)carbonyl]amino}piperidin-1- | | |
| | yl)-4-{[(2-methoxyethyl)amino]carbonyl}- | | |
| | 1,3-thiazole-5- | | |
| | carboxylic acid | | |
| 104 | 2-[(3S,4R)-4-{[(3,4-Dichloro-5- | LC/MS (ES⁺) (M + H)⁺: 585, 587 for | Example 88 |
| | methyl-1H-pyrrol-2- | C₂₂H₂₆Cl₂N₈O₅S | |
| | yl)carbonyl]amino}-3-(4-methyl- | NMR: 1.92 (m, 1 H), 2.04 (m, 1 H), | |
| | 1H-1,2,3-triazol-1-yl)piperidin-1- | 2.11 (s, 3 H), 2.12 (s, 3 H), 3.19 (s, | |
| | yl]-4-{[(2-methoxyethyl)amino]carbonyl}- | 3 H), 3.46 (m, 4 H), 3.56 (m, 1 H), | |
| | 1,3-thiazole-5- | 3.96 (m, 2 H), 4.41 (m, 1 H), | |
| | carboxylic acid | 4.61 (m, 1 H), 5.16 (m, 1 H), 7.24 (d, 1 | |
| | | H), 7.98 (s, 1 H), 9.33 (s, 1 H), | |
| | | 12.02 (s, 1 H) | |
| 105 | 2-[(3S,4R)-4-{[(3,4-Dichloro-5- | LC/MS (ES⁺) (M + H)⁺: 629, 631 for | Example 89 |
| | methyl-1H-pyrrol-2- | C₂₄H₃₀ClyN₈O₆S | |
| | yl)carbonyl]amino}-3-(4-methyl- | NMR: 1.90 (m, 1 H), 2.08 (m, 1 H), | |
| | 1H-1,2,3-triazol-1-yl)piperidin-1- | 2.11 (s, 3 H), 2.12 (s, 3 H), 3.19 (m, | |
| | yl]-4-({[2-methoxy-1- | 6 H), 3.48 (m, 5 H), 3.97 (m, 2 H), | |
| | (methoxymethyl)ethyl]amino}carbonyl)- | 4.23 (m, 2 H), 4.63 (m, 1 H), | |
| | 1,3-thiazole-5- | 5.08 (m, 1 H), 7.07 (d, 1 H), 7.86 (d, 1 | |
| | carboxylic acid | H), 8.82 (s, 1 H), 12.09 (s, 1 H) | |
| 106 | 2-[(3S,4R)-4-{[(3,4-Dichloro-5- | LC/MS (ES⁺) (M + H)⁺: 673, 675 for | Example 90 |
| | methyl-1H-pyrrol-2- | C₂₆H₃₄Cl₂N₈O₇S | |
| | yl)carbonyl]amino}-3-(4-methyl- | NMR: 1.96 (m, 1 H), 2.11 (s, 3 H), | |
| | 1H-1,2,3-triazol-1-yl)piperidin-1- | 2.12 (s, 3 H), 2.18 (m, 1 H), 3.21 (s, | |
| | yl]-4-({[2-methoxy-1,1- | 9 H), 3.55 (s, 6 H), 3.55 (m, 1 H), | |
| | bis(methoxymethyl)ethyl]amino}carbonyl)- | 3.92 (m, 2 H), 4.28 (m, 1 H), | |
| | 1,3-thiazole-5- | 4.63 (m, 1 H), 5.10 (m, 1 H), 7.04 (d, 1 | |
| | carboxylic acid | H), 7.87 (s, 1 H), 8.22 (s, 1 H), | |
| | | 12.09 (s, 1 H) | |
| 107 | 2-((3S,4R)-3-(4-Chloro-1H-1,2,3- | LC/MS (ES⁺) (M + H)⁺: 504, 506, | Example 71 |
| | triazol-1-yl)-4-{[(3,4-dichloro-5- | 508 for C₁₇H₁₆Cl₃N₇O₃S | |
| | methyl-1H-pyrrol-2- | NMR: 1.98 (m, 1 H), 2.16 (m, 1 H), | |
| | yl)carbonyl]amino}piperidin-1- | 2.16 (s, 3 H), 3.41 (m, 1 H), | |
| | yl)-1,3-thiazole-5-carboxylic acid | 3.96 (m, 2 H), 4.34 (m, 1 H), 4.78 (m, 1 | |
| | | H), 5.24 (m, 1 H), 7.17 (d, 1 H), | |
| | | 7.76 (s, 1 H), 8.38 (s, 1 H), | |
| | | 12.02 (s, 1 H), 12.73 (s, 1 H) | |
| 108 | 2-((3S,4R)-3-(4-Chloro-1H-1,2,3- | LC/MS (ES⁺) (M + H)⁺: 605, 607, | Example 85 |
| | triazol-1-yl)-4-{[(3,4-dichloro-5- | 609 for C₂₁H₂₃Cl₃N₈O₅S | |
| | methyl-1H-pyrrol-2- | NMR: 1.92 (m, 1 H), 2.10 (s, 3 H), | |
| | yl)carbonyl]amino}piperidin-1- | 2.18 (m, 1 H), 3.19 (s, 3 H), | |
| | yl)-4-{[(2-methoxyethyl)amino]carbonyl}- | 3.43 (m, 4 H), 3.54 (m, 1 H), 3.79 (m, 1 | |
| | 1,3-thiazole-5- | H), 5.16 (m, 1 H), 7.14 (d, 1 H), | |
| | carboxylic acid | 8.37 (s, 1 H), 9.38 (s, 1 H), | |
| | | 12.02 (s, 1 H) | |
| 109 | 2-((3S,4R)-3-(4-Chloro-1H-1,2,3- | LC/MS (ES⁺) (M + H)⁺: 649, 651, | Example 86 |
| | triazol-1-yl)-4-{[(3,4-dichloro-5- | 653 for C₂₃H₂₇Cl₃N₈O₆S | |
| | methyl-1H-pyrrol-2- | NMR: 1.93 (m, 1 H), 2.10 (s, 3 H), | |
| | yl)carbonyl]amino}piperidin-1- | 2.19 (m, 1 H), 3.19 (s, 6 H), | |
| | yl)-4-({[2-methoxy-1- | 3.42 (m, 4 H), 4.08 (m, 2 H), 4.25 (m, 1 | |
| | (methoxymethyl)ethyl]amino}carbonyl)- | H), 4.41 (m, 1 H), 4.63 (m, 1 H), | |
| | 1,3-thiazole-5-carboxylic | 5.16 (m, 1 H), 7.12 (d, 1 H), | |
| | acid | 8.37 (s, 1 H), 8.92 (s, 1 H), 12.01 (s, 1 | |
| | | H) | |
| 110 | 2-((3S,4R)-3-(4-Chloro-1H-1,2,3- | LC/MS (ES⁺) (M + H)⁺: 587, 589, | Example 87 |
| | triazol-1-yl)-4-{[(3,4-dichloro-5- | 591 for C₂₁H₂₁Cl₃N₈O₄S | |
| | methyl-1H-pyrrol-2- | NMR: 0.67 (m, 4 H), 1.91 (m, 1 H), | |
| | yl)carbonyl]amino}piperidin-1- | 2.11 (s, 3 H), 2.18 (m, 1 H), | |
| | yl)-4-[(cyclopropylamino)carbonyl]- | 2.83 (m, 1 H), 3.57 (m, 1 H), 3.93 (m, 2 | |
| | 1,3-thiazole-5- | H), 4.46 (m, 1 H), 4.65 (m, 1 H), | |
| | carboxylic acid | 5.12 (m, 1 H), 7.14 (d, 1 H), | |
| | | 8.36 (s, 1 H), 9.18 (m, 1 H), 12.01 (s, 1 | |
| | | H) | |
| 111 | 2-((3S,4R)-3-[4-(Cyanomethyl)- | LC/MS (ES⁺) (M + H)⁺: 654, 656 for | Example 93 |
| | 1H-1,2,3-triazol-1-yl]-4-{[(3,4- | C₂₅H₂₉Cl₂N₉O₆S | |
| | dichloro-5-methyl-1H-pyrrol-2- | NMR: 1.92 (m, 1 H), 2.11 (s, 3 H), | |
| | yl)carbonyl]amino}piperidin-1- | 2.19 (m, 1 H), 3.29 (s, 6 H), | |
| | yl)-4-({[2-methoxy-1- | 3.41 (m, 5 H), 4.01 (m, 2 H), 4.07 (s, 2 | |
| | (methoxymethyl)ethyl]amino}carbonyl)- | H), 4.32 (m, 1 H), 4.46 (m, 1 H), | |
| | 1,3-thiazole-5- | 5.24 (m, 1 H), 7.15 (d, 1 H), | |
| | carboxylic acid | 8.15 (s, 1 H), 8.95 (d, 1 H), 8.95 (d, 1 | |
| | | H), 12.01 (s, 1 H) | |
| 112 | 2-{(3S,4R)-4-{[(3,4-Dichloro-5- | LC/MS (ES⁺) (M + H)⁺: 647, 649 for | Example 98 |
| | methyl-1H-pyrrol-2- | C₂₄H₂₉Cl₂FN₈O₆S | |
| | yl)carbonyl]amino}-3-[4- | NMR: 1.96 (m, 1 H), 2.11 (m, 1 H), | |
| | (fluoromethyl)-1H-1,2,3-triazol- | 2.19 (s, 3 H), 3.18 (s, 6 H), 3.42 (m, | |
| | 1-yl]piperidin-1-yl}-4-({[2- | 5 H), 4.08 (m, 2 H), 4.26 (m, 1 H), | |
| | methoxy-1-(methoxymethyl)ethyl]amino}carbonyl)- | 4.43 (m, 1 H), 4.63 (m, 1 H), | |
| | 1,3- | 5.27 (m, 1 H), 5.28 (m, 1 H), 5.48 (m, 1 | |
| | thiazole-5-carboxylic acid | H), 7.16 (d, 1 H), 8.38 (s, 1 H), | |
| | | 8.97 (d, 1 H), 12.01 (s, 1 H) | |
| 113 | 2-((3S,4R)-3-(4-Bromo-1H-1,2,3- | LC/MS (ES⁺) (M + H)⁺: 694 for | Example 91 |
| | triazol-1-yl)-4-{[(3,4-dichloro-5- | C₂₃H₂₇BrCl₂N₈O₆S | |
| | methyl-1H-pyrrol-2- | NMR: 1.98 (m, 1 H), 2.11 (m, 1 H), | |
| | yl)carbonyl]amino}piperidin-1- | 2.19 (s, 3 H), 3.22 (s, 6 H), 3.47 (m, | |
| | yl)-4-({[2-methoxy-1- | 5 H), 4.08 (m, 2 H), 4.37 (m, 1 H), | |
| | (methoxymethyl)ethyl]amino}carbonyl)- | 4.47 (m, 1 H), 4.73 (m, 1 H), | |
| | 1,3-thiazole-5- | 5.26 (m, 1 H), 7.21 (d, 1 H), 8.42 (s, 1 | |
| | carboxylic acid | H), 8.93 (d, 1 H), 12.03 (s, 1 H) | |
| 114 | 2-{(3S,4R)-4-{[(3,4-Dichloro-5- | LC/MS (ES⁺) (M + H)⁺: 645, 647 for | Example 95 |
| | methyl-1H-pyrrol-2- | C₂₄H₃₀Cl₂N₈O₇S | |
| | yl)carbonyl]amino}-3-[4- | NMR: 1.99 (m, 1 H), 2.16 (m, 1 H), | |
| | (hydroxymethyl)-1H-1,2,3- | 2.16 (s, 3 H), 3.22 (s, 6 H), 3.49 (m, | |
| | triazol-1-yl]piperidin-1-yl}-4- | 5 H), 4.16 (m, 2 H), 4.34 (m, 1 H), | |
| | ({[2-methoxy-1- | 4.51 (m, 1 H), 4.54 (s, 2 H), | |
| | (methoxymethyl)ethyl]amino}carbonyl)- | 4.78 (m, 1 H), 5.24 (m, 1 H), 7.15 (d, 1 | |
| | 1,3-thiazole-5- | H), 8.08 (s, 1 H), 8.99 (d, 1 H), | |
| | carboxylic acid | 12.03 (s, 1 H) | |
| 115 | 2-{(3S,4R)-4-{[(3,4-Dichloro-5- | LC/MS (ES⁺) (M + H)⁺: 601, 603 for | Example 94 |
| | methyl-1H-pyrrol-2- | C₂₂H₂₆Cl₂N₈O₆S | |
| | yl)carbonyl]amino}-3-[4- | NMR: 1.99 (m, 1 H), 2.07 (m, 1 H), | |
| | (hydroxymethyl)-1H-1,2,3- | 2.16 (s, 3 H), 3.21 (s, 3 H), 3.47 (m, | |
| | triazol-1-yl]piperidin-1-yl}-4- | 4 H), 3.63 (m, 1 H), 4.06 (m, 2 H), | |
| | {[(2-methoxyethyl)amino]carbonyl}- | 4.47 (m, 1 H), 4.54 (s, 2 H), | |
| | 1,3-thiazole-5- | 4.72 (m, 1 H), 5.27 (m, 1 H), 7.20 (d, 1 | |
| | carboxylic acid | H), 8.09 (s, 1 H), 9.38 (m, 1 H), | |
| | | 12.02 (s, 1 H) | |
| 116 | 2-{(3S,4R)-4-{[(3,4-Dichloro-5- | LC/MS (ES⁺) (M + H)⁺: 659, 661 for | Example 97 |
| | methyl-1H-pyrrol-2- | C₂₅H₃₂Cl₂N₈O₇S | |
| | yl)carbonyl]amino}-3-[4- | NMR: 1.93-2.03 (m, 1 H), 2.15 (s, | |
| | (methoxymethyl)-1H-1,2,3- | 3 H), 3.17 (s, 3 H), 3.24 (d, 6 H), | |
| | triazol-1-yl]piperidin-1-yl}-4- | 3.39-3.55 (m, 5 H), 4.08 (d, 1 H), | |
| | ({[2-methoxy-1- | 4.28-4.37 (m, 1 H), 4.40 (s, 2 H), | |
| | (methoxymethyl)ethyl]amino}carbonyl)- | 4.66-4.79 (m, 1 H), 5.22 (q, 1 H), | |
| | 1,3-thiazole-5- | 7.15 (d, 1 H), 8.14 (s, 1 H), 8.96 (d, | |
| | carboxylic acid | 1 H), 12.03 (s, 1 H) | |
| 117 | 2-{(3S,4R)-4-{[(3,4-Dichloro-5- | LC/MS (ES⁺) (M + H)⁺: 615, 617 for | Example 96 |
| | methyl-1H-pyrrol-2- | C₂₃H₂₈Cl₂N₈O₆S | |
| | yl)carbonyl]amino}-3-[4- | NMR: 1.93-2.04 (m, 1 H), 2.07 (s, | |
| | (methoxymethyl)-1H-1,2,3- | 1 H), 2.15 (s, 3 H), 3.17 (s, 3 H), | |
| | triazol-1-yl]piperidin-1-yl}-4- | 3.24 (s, 3 H), 3.43-3.49 (m, 4 H), | |
| | {[(2-methoxyethyl)amino]carbonyl}- | 3.52-3.63 (m, 1 H), 4.08 (dd, 1 H), | |
| | 1,3-thiazole-5- | 4.39 (s, 2 H), 5.21 (q, 1 H), 7.19 (d, | |
| | carboxylic acid | 1 H), 8.15 (s, 1 H), 9.37 (t, 1 H), | |
| | | 12.03 (s, 1 H) | |
| 118 | 2-[(3S,4R)-4-{[(3,4-Dichloro-5- | LC/MS (ES⁺) (M + H)⁺: 615, 617 for | Example 99 |
| | methyl-1H-pyrrol-2- | C₂₃H₂₈Cl₂N₈O₆S. | |
| | yl)carbonyl]amino}-3-(1H-1,2,3- | NMR: 2.01 (m, 1 H), 2.16 (s, 3 H), | |
| | triazol-1-yl)piperidin-1-yl]-4- | 2.23 (m, 1 H), 3.24 (s, 6 H), | |
| | ({[2-methoxy-1- | 3.43 (m, 5 H), 4.02 (m, 2 H), 4.36 (m, 1 | |
| | (methoxymethyl)ethyl]amino}carbonyl)- | H), 4.52 (m, 1 H), 4.71 (m, 1 H), | |
| | 1,3-thiazole-5- | 5.21 (m, 1 H), 7.16 (d, 1 H), | |
| | carboxylic acid | 7.75 (s, 1 H), 8.12 (s, 1 H), 8.91 (d, 1 H), | |
| | | 12.03 (s, 1 H) | |

### Example 119

### cis(±)2-[4-{[(3,4-Dichloro-5-methyl-1H-pyrrol-2-yl)carbonyl]amino}-3-(1H-1,2,3-triazol-1-yl)piperidin-1-yl]-1,3-thiazole-5-carboxylic acid

cis(±)Methyl 2-[4-{[(3,4-dichloro-5-methyl-1H-pyrrol-2-yl)carbonyl]amino}-3-(1H-1,2,3-triazol-1-yl)piperidin-1-yl]-1,3-thiazole-5-carboxylate (Example 126, 18 mg) was dissolved in a mixture of methanol (3 mL) and THF (1 mL), to which was added barium hydroxide (21 mg) as a suspension in water (2 mL). The reaction was stirred overnight at room temperature. The reaction mixture was concentrated in vacuo to remove the THF and methanol, cooled to 0° C., neutralized with 2N HCl, filtered and rinsed with deionized water, yielding the title compound (11 mg, 61.1%). MS (ES) (M+H)⁺: 470, 472 for C₁₇H₁₇Cl₂N₇O₃S; NMR: 1.97 (m, 1H), 2.14 (m, 1H), 2.15 (s, 3H), 3.57 (m, 1H), 3.95 (m, 1H), 4.00 (dd, 1H), 4.34 (dd, 1H), 4.73 (m, 1H), 5.22 (q, 1H), 7.10 (d, 1H), 7.71 (s, 1H), 7.72 (d, 1H), 8.09 (d, 1H), 12.01 (s, 1H), 12.68 (s, 1H).

### Example 120

### 2-[(3S,4R)-4-{[(3,4-Dichloro-5-methyl-1H-pyrrol-2-yl)carbonyl]amino}-3-(1H-1,2,3-triazol-1-yl)piperidin-1-yl]-N-methoxy-1,3-thiazole-5-carboxamide

2-[(3S,4R)-4-{[(3,4-Dichloro-5-methyl-1H-pyrrol-2-yl)carbonyl]amino}-3-(1H-1,2,3-triazol-1-yl)piperidin-1-yl]-1,3-thiazole-5-carboxylic acid (Example 101, 46 mg) was dissolved in anhydrous NMP (2 mL) under an argon atmosphere, followed by the addition of HATU (41 mg) and DIEA (54 μL) and the reaction was stirred at room temperature for forty minutes. Methoxylamine hydrochloride (9 mg) was added, and the reaction was stirred overnight at room temperature. The reaction mixture was then added slowly via pipette to rapidly stirring water (40 mL), cooled to 0° C., filtered and rinsed with deionized water. The crude product was purified by supercritical fluid chromatography (SFC), yielding the title compound as a beige solid (6 mg, 11.8%). MS (ES) (M+H)⁺: 499, 501 for C₁₈H₂₀Cl₂N₈O₃S; NMR: 1.97 (m, 1H), 2.11 (m, 1H), 2.15 (s, 3H), 3.55 (m, 1H), 3.64 (s, 3H), 3.92 (m, 1H), 3.98 (dd, 1H), 4.32 (dd, 1H), 4.72 (m, 1H), 5.21 (q, 1H), 7.11 (d, 1H), 7.67 (s, 1H), 7.72 (s, 1H), 8.08 (s, 1H), 11.35 (broad s, 1H), 12.01 (s, 1H).

### Example 121

### cis(±)Methyl 2-[4-{[(3,4-dichloro-5-methyl-1H-pyrrol-2-yl)carbonyl]amino}-3-(4-methyl-1H-1,2,3-triazol-1-yl)piperidin-1-yl]-1,3-thiazole-5-carboxylate

cis(±)Methyl 2-(3-amino-4-{[(3,4-dichloro-5-methyl-1H-pyrrol-2-yl)carbonyl]amino}piperidin-1-yl)-1,3-thiazole-5-carboxylate (Example 67, 125 mg) was dissolved in anhydrous THF (5 mL) under an argon atmosphere. N′-[2,2-dichloro-1-methylethylidene]-4-methylbenzenesulfonohydrazide (130 mg) (prepared according to procedure in Bulletin of the Chemical Society of Japan (1986), 59(1), 179-83) was added, followed by DIEA (0.18 mL), and the reaction was stirred overnight at room temperature. The reaction was diluted with EtOAc (750 mL) and washed with saturated aqueous NaHCO₃ (75 mL), brine (50 mL), dried over anhydrous MgSO₄ and concentrated in vacuo. The crude product was then purified by silica gel chromatography (0.5-5% CH₃OH in DCM), and then recrystallized from CH₃CN to yield the title compound as a light yellow solid (32 mg, 22.2%). MS (ES) (M+H)⁺: 498, 500 for C₁₉H₂₁Cl₂N₇O₃S; NMR: 1.95 (m, 1H), 2.09 (m, 1H), 2.15 (s, 3H), 2.18 (s, 3H), 3.56 (m, 1H), 3.73 (s, 3H), 3.97 (dd, 2H), 4.34 (dd, 1H), 4.69 (m, 1H), 5.12 (q, 1H), 7.09 (d, 1H), 7.80 (broad s, 1H), 7.82 (s, 1H), 12.02 (s, 1H).

### Example 122

### cis(±)₂-[4-{[(3,4-Dichloro-5-methyl-1H-pyrrol-2-yl)carbonyl]amino}-3-(4-methyl-1H-1,2,3-triazol-1-yl)piperidin-1-yl]-1,3-thiazole-5-carboxylic acid

The title compound was prepared in a manner analogous to (Example 119) starting from cis(±)methyl 2-[4-{[(3,4-dichloro-5-methyl-1H-pyrrol-2-yl)carbonyl]amino}-3-(4-methyl-1H-1,2,3-triazol-1-yl)piperidin-1-yl]-1,3-thiazole-5-carboxylate (Example 121). MS (ES) (M+H)⁺: 484, 486 for C₁₈H₁₉Cl₂N₇O₃S; NMR: 1.95 (m, 1H), 2.09 (m, 1H), 2.15 (s, 3 H), 2.18 (s, 3H), 3.54 (m, 1H), 3.95 (dd, 2H), 4.32 (dd, 1H), 4.68 (m, 1H), 5.12 (q, 1H), 7.11 (d, 1H), 7.73 (s, 1H), 7.81 (s, 1H), 12.02 (s, 1H), 12.69 (s, 1H).

### Example 123

### Ethyl 2-[(3S,4R)-4-{[(3,4-dichloro-5-methyl-1H-pyrrol-2-yl)carbonyl]amino}-3-(1H-1,2,3-triazol-1-yl)piperidin-1-yl]-1,3-benzothiazole-7-carboxy late

3,4-Dichloro-5-methyl-N-[(3S,4R)-3-(1H-1,2,3-triazol-1-yl)piperidin-4-yl]-1H-pyrrole-2-carboxamide hydrobromide (Intermediate 91), 135 mg) was placed in a microwave vial, followed by ethyl 2-bromobenzothiazole-7-carboxylate (WO 2006/087543 A1), 153 mg), anhydrous NMP (2 mL), and DIEA (0.25 mL). The reaction was heated at 125° C. for 40 minutes on the microwave, then diluted with EtOAc (100 mL) and washed with saturated aqueous NaHCO₃ (75 mL), brine (50 mL), dried over anhydrous MgSO₄ and concentrated in vacuo. The crude product was then purified by silica gel chromatography (0.5-5% CH₃OH in DCM), yielding the title compound as a beige solid (0.12 g, 61.5%). MS (ES) (M+H)⁺: 548, 550 for C₂₃H₂₃Cl₂N₇O₃S; NMR: 1.34 (t, 3H), 2.02 (m, 1H), 2.15 (s, 3H), 2.19 (m, 1H), 3.66 (m, 1H), 4.11 (dd, 1H), 4.16 (m, 1H), 4.37 (q, 2H), 4.42 (m, 1H), 4.76 (m, 1H), 5.26 (q, 1H), 7.11 (d, 1H), 7.42 (t, 1H), 7.67 (dd, 1H), 7.71 (d, 1H), 7.71 (dd, 1H), 8.12 (d, 1H), 12.03 (s, 1H).

### Example 124

### 2-[(3S,4R)-4-{[(3,4-Dichloro-5-methyl-1H-pyrrol-2-yl)carbonyl]amino}-3-(1H-1,2,3-triazol-1-yl)piperidin-1-yl]-1,3-benzothiazole-7-carboxylic acid

Ethyl 2-[(3S,4R)-4-{[(3,4-dichloro-5-methyl-1H-pyrrol-2-yl)carbonyl]amino}-3-(1H-1,2,3-triazol-1-yl)piperidin-1-yl]-1,3-benzothiazole-7-carboxylate (Example 123, 0.1 g) was dissolved in anhydrous CH₃OH (4 mL), followed by the addition of barium (II) hydroxide (27 mg) as a suspension in water (2.5 mL) and the reaction was stirred at room temperature for eighteen hours. The reaction was then cooled to 0° C. and neutralized with the dropwise addition of 2N HCl, filtered and rinsed with deionized water. The crude product was then recrystallized from methanol, yielding the title compound as an off-white powder (70 mg, 73.7%). MS (ES) (M+H)⁺: 520, 522 for C₂₁H₁₉Cl₂N₇O₃S; NMR: 2.02 (m, 1H), 2.14 (m, 1H), 2.15 (s, 3H), 3.66 (m, 1H), 4.11 (dd, 1H), 4.15 (m, 1H), 4.43 (m, 1H), 4.76 (m, 1H), 5.26 (q, 1H), 7.12 (d, 1H), 7.39 (t, 1H), 7.65 (dd, 1H), 7.68 (dd, 1H), 7.71 (d, 1H), 8.11 (d, 1H), 12.02 (s, 1H), 13.50 (broad s, 1H).

### Example 125

### 2-[(3S,4R)-4-{[(3,4-Dichloro-5-methyl-1H-pyrrol-2-yl)carbonyl]amino}-3-(1H-1,2,3-triazol-1-yl)piperidin-1-yl]-N-methoxy-1,3-benzothiazole-7-carboxamide

2-[(3S,4R)-4-{[(3,4-Dichloro-5-methyl-1H-pyrrol-2-yl)carbonyl]amino}-3-(1H-1,2,3-triazol-1-yl)piperidin-1-yl]-1,3-benzothiazole-7-carboxylic acid (Example 124, 34 mg) was dissolved in anhydrous DMF (3 mL) under an argon atmosphere, followed by the addition of HATU (30 mg) and DIEA (28 μL) and the reaction was stirred at room temperature for forty minutes. Methoxylamine hydrochloride (7 mg) was added, and the reaction was stirred overnight at room temperature. The reaction mixture was then diluted with EtOAc (125 mL) and washed with saturated aqueous NH₄Cl (75 mL), saturated aqueous NaHCO₃ (60 mL), brine (40 mL), dried over anhydrous MgSO₄ and concentrate in vacuo. The crude product was purified by silica gel column using 0.5-5% CH₃OH in DCM, yielding the title compound as a yellow solid (15 mg, 41.7%). MS (ES) (M+H)⁺: 549, 551 for C₂₂H₂₁Cl₂N₈O₃S; NMR: 2.02 (m, 1H), 2.14 (m, 1H), 2.15 (s, 3H), 3.65 (m, 1H), 3.72 (s, 3H), 4.06-4.16 (m, 2H), 4.42 (dd, 1H), 4.75 (m, 1H), 5.25 (q, 1H), 7.12 (d, 1H), 7.35 (t, 1H), 7.50 (d, 1H), 7.57 (d, 1H), 7.71 (d, 1H), 8.11 (d, 1H), 12.03 (s, 1H).

### Example 126

### cis(±)Methyl 2-[4-{[(3,4-dichloro-5-methyl-1H-pyrrol-2-yl)carbonyl]amino}-3-(1H-1,2,3-triazol-1-yl)piperidin-1-yl]-1,3-thiazole-5-carboxylate

cis(±)Methyl 2-(3-azido-4-{[(3,4-dichloro-5-methyl-1H-pyrrol-2-yl)carbonyl]amino}piperidin-1-yl)-1,3-thiazole-5-carboxylate (Example 65, 66 mg) was placed under an argon atmosphere, followed by the addition of bicyclo[2.2.1]hepta-2,5-diene, and the reaction was placed in an oil bath preheated to 120° C., and stirred at this temperature for two hours. The mixture was diluted with hexanes (35 mL), filtered and washed with hexanes. The resultant crude product was purified by reverse-phase chromatography, then recrystallized from methanol, yielding the title compound (9 mg, 13%). MS (ES) (M+H)⁺: 484, 486 for C₁₈H₁₉Cl₂N₇O₃S; NMR: 1.98 (m, 1H), 2.15 (s, 3H), 2.18 (m, 1H), 3.59 (m, 1 H), 3.73 (s, 3H), 3.96-4.04 (m, 2H), 4.35 (dd, 1H), 4.74 (m, 1H), 5.22 (q, 1H), 7.09 (d, 1H), 7.72 (d, 1H), 7.81 (s, 1H), 8.08 (d, 1H), 12.02 (s, 1H).

### Preparation of Starting Materials

### Intermediate 1

### 4-Ethyl 1-methyl 3-oxopiperidine-1,4-dicarboxylate

To a solution of 1-benzyl-4-(ethoxycarbonyl)-3-oxopiperidinium chloride (25.38 g, 85 mmol) in a 1:1 mixture of absolute ethanol and water (300 mL) was added 10% palladium on activated carbon (50% wet with water) (4.0 g). The flask was transferred to a parr shaker where it was evacuated and backfilled with nitrogen several times before evacuating and backfilling with hydrogen. The reaction was run on the parr shaker for two days with positive hydrogen pressure. The crude reaction mixture was bubbled with nitrogen for 20 minutes, then diatomaceous earth was added and the reaction mixture was filtered through diatomaceous earth. After washing through several times with ethanol, solvent was removed under reduced pressure. The residue was taken up in water and cooled to 0° C. A cold solution of potassium carbonate (35.2 g, 255 mmol) in water (10 mL) was added followed by the dropwise addition of methyl chloroformate (16.8 mL, 217 mmol). After stirring at 0° C. for 30 minutes the reaction was warmed to room temperature and stirred for one hour. The reaction mixture was extracted with ether (×3) and the organic extracts were dried with MgSO₄ and concentrated to a red oil. Kugelrohr distillation afforded product was a colourless oil which solidified to colourless crystals after several days in the refrigerator (15 g, 77%) MS (ES) (M+H)⁺: 230 for C₁₀H₁₅NO₅; NMR: 1.22 (t, 3H), 2.23 (t, 2H), 3.45 (t, 2H), 3.61 (s, 4H), 4.00 (s, 2H), 4.19 (q, 2H), 11.92 (s, 1H).

### Intermediate 2

### 10-Ethyl 7-methyl 1,4-dioxa-7-azaspiro[4.5]decane-7,10-dicarboxylate

To a solution of 4-ethyl 1-methyl 3-oxopiperidine-1,4-dicarboxylate (4.0 g, 17 mmol, Intermediate 1) in benzene (300 mL) was added ethylene glycol (50 mL) and p-toluenesulfonic acid (0.35 g). The reaction was heated to reflux with azeotropic removal of water for 5 days. After cooling to room temperature, the reaction mixture was washed with saturated aqueous sodium carbonate and brine, then dried with MgSO₄ and concentrated to a colourless oil which was then purified by silica gel flash column (gradient elution 0-50% EtOAc in DCM). Pure fractions were combined to yield product as a colourless oil (3.8 g, 83%). NMR: 1.25-1.32 (m, 3H), 1.63 (s, 1H), 1.92-2.07 (m, 2H), 2.80 (dd, 1H), 3.16-3.31 (m, 2H), 3.71 (s, 3H), 3.82 (dt, 2H), 3.94-4.08 (m, 3H), 4.19 (q, 2H).

### Intermediate 3

The following Intermediate was prepared by the procedure described in Intermediate 2 from the starting materials (SM) indicated.

| Int | Compound | Data | SM |
|---|---|---|---|
| 3 | 11-Ethyl 8-methyl 1,5-dioxa-8- | NMR: 1.18 (t, 3 H), 1.50 (s, 1 | Intermediate 1 |
| | azaspiro[5.5]undecane-8,11- | H), 1.61-1.75 (m, 3 H), 2.89 (s, | and 1,3- |
| | dicarboxylate | 1 H), 3.46 (s, 2 H), 3.60 (s, 4 H), | propanediol |
| | | 3.78-3.94 (m, 4 H), | |
| | | 3.99-4.12 (m, 2 H) | |

### Intermediate 4

### 4-Ethyl 1-methyl 3,3-dimethoxypiperidine-1,4-dicarboxylate

A solution of 3 g (13 mmol) of 4-ethyl 1-methyl 3-oxopiperidine-1,4-dicarboxylate (Intermediate 1), trimethylorthoformate and 200 mg p-toluenesulfonic acid in 300 mL MeOH was heated at reflux overnight. The solution was diluted with aqueous Na₂CO₃ and extracted 2 times with EtOAc, which was washed with brine. Drying (MgSO₄) and removal of solvent gave 3.4 g of product as an oil. NMR (CDCl₃): 1.34 (t, 3H), 1.74 (m, 1H), 1.93 (m, 1H), 3.09 (m, 1H), 3.25 (2s, 6H), 3.32-3.44 (m, 2H), 3.76 (s, 3H), 3.97 (m, 1H), 4.02-4.38 (m, 3H).

### Intermediate 5

### 7-(Methoxycarbonyl)-1,4-dioxa-7-azaspiro[4.5]decane-10-carboxylic acid

To a suspension of 10-ethyl 7-methyl 1,4-dioxa-7-azaspiro[4.5]decane-7,10-dicarboxylate (2.1 g, 10.6 mmol, Intermediate 2) in methanol (50 mL) was added barium hydroxide (3.6 g, 21.2 mmol) and water (10 mL). After stirring at room temperature over the weekend, the solution was acidified with 1N HCl to pH 3 and then saturated with sodium chloride. The aqueous layer was partitioned with EtOAc (×3) and the organic portion was dried with MgSO₄ and concentrated to a colourless oil (2.4 g, 93%). NMR: 1.71-1.81 (m, 2H), 2.72 (t, 1H), 3.11 (s, 2H), 3.33 (s, 1H), 3.58 (s, 3H), 3.64 (d, 2H), 3.81-3.96 (m, 3H), 12.24 (s, 1H).

### Intermediates 6-7

The following Intermediates were prepared by the procedure described in Intermediate from the starting materials (SM) indicated.

| Int | Compound | Data | SM |
|---|---|---|---|
| 6 | 8-(Methoxycarbonyl)-1,5-dioxa- | NMR: 1.51 (s, 1 H), 1.68 (d, 3 H), | Intermediate 3 |
| | 8-azaspiro[5.5]undecane-11- | 2.83 (s, 1 H), 3.44 (s, 2 H), | |
| | carboxylic acid | 3.57-3.62 (m, 3 H), 3.69 (s, 2 H), 3.86 (s, | |
| | | 4 H) | |
| 7 | 3,3-Dimethoxy-1- | NMR (CDCl₃): 1.83-2.06 (m, 2 H), | Intermediate 4 |
| | (methoxycarbonyl)piperidine-4- | 3.04 (m, 1 H), 3.25 (s, 3 H), 3.37 (s, | |
| | carboxylic acid | 3 H), 3.32-3.52 (m, 2 H), 3.73 (s, 3 | |
| | | H), 3.82-4.09 (m, 2 H) | |

### Intermediate 8

### Methyl 10-{[(benzyloxy)carbonyl]amino}-1,4-dioxa-7-azaspiro[4.5]decane-7-carboxylate

Ethyl chloroformate (0.74 mL, 7.8 mmol) and triethylamine (1.2 mL, 8.5 mmol) were added to a cold solution of 7-(methoxycarbonyl)-1,4-dioxa-7-azaspiro[4.5]clecane-10-carboxylic acid (1.75 g, 7.1 mmol, Intermediate 5) in dry acetone (50 mL) at 0° C. After stirring at 0° C. for one hour, a solution of sodium azide (1.2 g, 18 mmol) in water (20 mL) was added. The reaction was then stirred for an additional 1.5 hours at 0° C. and then overnight at room temperature. Acetone was removed under reduced pressure and the residue was extracted with toluene (×3), which was, in turn, dried with MgSO₄ and concentrated by heating to reflux with a dean stark trap. Once the volume was ˜100 mL, benzyl alcohol (1.1 mL, 10.6 mmol) was added and the reaction was heated to reflux overnight. After cooling to room temperature, solvent was removed and the residue was diluted with DCM and then washed with 1N HCl (×3), dried with MgSO₄ and concentrated to a yellow oil. Purification by silica gel flash column (gradient elution 0-30% EtOAc in DCM) followed by concentrating pure fractions yielded product as a white solid (1.08 g, 43%). NMR: 1.91 (s, 1H), 2.72 (s, 2H), 2.86 (s, 1 H), 3.69 (s, 3H), 3.93-4.08 (m, 4H), 4.13 (s, 1H), 4.89 (s, 1H), 5.11 (s, 2H), 7.30-7.39 (m, 5H).

### Intermediate 9-10

The following Examples were prepared by the procedure described in Intermediate 8 from the starting materials (SM) indicated.

| Int | Compound | Data | SM |
|---|---|---|---|
| 9 | Methyl 11-{[(benzyloxy)carbonyl]amino}- | NMR: 1.45 (s, 2 H), 1.62 (s, 1 H), | Intermediate 6 |
| | 1,5-dioxa- | 1.77 (s, 1 H), 3.13 (s, 2 H), 3.33 (s, 3 H), | |
| | 8-azaspiro[5.5]undecane-8- | 3.59 (d, 3 H), 3.79 (s, 2 H), 3.90 (s, 3 H), | |
| | carboxylate | 4.99-5.07 (m, 2 H), 7.31-7.38 (m, 5 H) | |
| 10 | Methyl 4-{[(benzyloxy)carbonyl]amino}- | NMR (CDCl₃): 1.89 (m, 2 H), 3.27 (s, 3 | Intermediate 7 |
| | 3,3- | H), 3.33 (m, 3 H), 3.38-3.52 (m, 4 H), | |
| | dimethoxypiperidine-1- | 3.75 (s, 3 H), 3.91 (m, 1 H), 5.16 (s, 2 | |
| | carboxylate | H), 7.45 (s, 5 H) | |

### Intermediate 11

### Methyl 10-amino-1,4-dioxa-7-azaspiro[4.5]decane-7-carboxylate

To a solution of methyl 10-{[(benzyloxy)carbonyl]amino}-1,4-dioxa-7-azaspiro[4.5]decane-7-carboxylate (1.08 g, 3 mmol, Intermediate 8) in absolute ethanol (100 mL) was added 10% activated palladium on carbon (50% wet with water)(0.30 g). The flask was evacuated and backfilled with nitrogen several times then evacuated and equipped with a balloon filled with hydrogen gas. After stirring at room temperature under hydrogen atmosphere, the balloon was removed and the reaction mixture was bubbled with nitrogen gas for 20 minutes. The crude reaction mixture was filtered through diatomaceous earth and solvent was evaporated to obtain a white solid (0.72 g). NMR: 1.27-1.42 (m, 1H), 1.68 (ddd, 1H), 1.77 (s, 1H), 2.77 (dd, 2H), 2.99 (s, 1H), 3.54-3.59 (m, 3H), 3.69 (s, 2H), 3.71 (d, 1H), 3.81 (d, 1H), 3.84-4.00 (m, 4H).

### Intermediates 12-13

The following Intermediates were prepared by the procedure described in Intermediate 11 from the starting materials (SM) indicated.

| Int | Compound | Data | SM |
|---|---|---|---|
| 12 | Methyl 11-amino-1,5-dioxa-8- | NMR: 1.25-1.80 (m, 4 H), 2.73 (s, | Intermediate 9 |
| | azaspiro[5.5]undecane-8- | 1 H), 3.04-3.19 (m, 3 H), 3.33 (s, 2 | |
| | carboxylate | H), 3.54-3.66 (m, 4 H), 3.79 (s, 2 | |
| | | H), 3.94 (s, 2 H) | |
| 13 | Methyl 4-amino-3,3- | NMR (CDCl₃): 1.33-1.58 (m, 2 H), | Intermediate |
| | dimethoxypiperidine-1- | 1.53-1.75 (m, 1 H), 3.09-3.22 (m, 8 | 10 |
| | carboxylate | H), 3.64 (s, 3 H), 3.67-4.00 (m, 2 H) | |

### Intermediate 14

### Methyl 10-{[(3,4-dichloro-5-methyl-1H-pyrrol-2-yl)carbonyl]amino}-1,4-dioxa-7-azaspiro[4.5]decane-7-carboxylate

A solution of 3,4-dichloro-5-methyl-1H-pyrrole-2-carboxylic acid (0.45 g, 2.3 mmol, Motekaitis, R. J.; Heinert, D. H.; Martell, Arthur E. J. Org. Chem. 35(8), 2504 (1970)), methyl 10-amino-1,4-dioxa-7-azaspiro[4.5]decane-7-carboxylate (0.50 g, 2.3 mmol, Intermediate 11), HOBt (0.31 g, 2.3 mmol) and NMM (0.99 mL, 8.1 mmol) in DCM (100 mL) was stirred at room temperature for one hour, after which EDC (0.79 g, 4.1 mmol) was added. After stirring at room temperature for 12 hours, the crude reaction mixture was washed with saturated aqueous sodium bicarbonate (×2), 1N HCl (×2), water (×2), and brine (×1). The organic portion was then dried with MgSO₄ and concentrated to a tan solid. Trituration with ether yielded a white solid (0.59 g, 66%). MS (ES) (M+H)⁺: 392 for C₁₅H₁₉Cl₂N₃O₅; NMR: 1.53 (dd, J=12.43, 4.14 Hz, 1H), 1.79-1.91 (m, 1H), 2.15-2.20 (m, 3H), 2.85 (s, 1H), 2.95 (s, 1H), 3.57-3.61 (m, 3H), 3.73-3.82 (m, 1H), 3.84-3.98 (m, 3H), 3.99-4.08 (m, 1H), 4.16 (s, 1H), 4.22-4.33 (m, 1H), 6.91 (d, J=8.85 Hz, 1H), 12.16 (s, 1H).

### Intermediates 15-16

The following Intermediates were prepared by the procedure described in Intermediate 14 from the starting materials (SM) indicated.

| Int | Compound | Data | SM |
|---|---|---|---|
| 15 | Methyl 11-{[(3,4- | MS (ES) (M + H)⁺: 406 for C₁₆H₂₁Cl₂N₃O₅ | Intermediate |
| | dichloro-5-methyl-1H- | NMR: 1.47 (s, 1 H), 1.69 (s, 1 H), 1.80 (s, 2 | 12 |
| | pyrrol-2-yl)carbonyl]amino}- | H), 2.13-2.21 (m, 3 H), 2.75 (s, 1 H), | |
| | 1,5-dioxa-8- | 2.96 (s, 1 H), 3.55-3.66 (m, 3 H), 3.79 (s, 3 H), | |
| | azaspiro[5.5]undecane-8- | 3.90-4.06 (m, 4 H), 7.16 (d, 1 H), | |
| | carboxylate | 12.15 (s, 1 H) | |
| 16 | Methyl 4-{[(3,4- | MS (ES) (M − H)⁻: 392 for C₁₅H₂₁Cl₂N₃O₅; | Intermediate |
| | dichloro-5-methyl-1H- | NMR: 1.76 (m, 2 H), 2.24 (s, 3 H), 3.15 (s, | 13 |
| | pyrrol-2-yl)carbonyl]amino}- | 3 H), 3.28 (s, 3 H), 3.31 (m, 2 H), 3.52 (m, | |
| | 3,3-dimethoxypiperidine- | 2 H), 3.55 (m, 1 H), 3.63 (s, 3 H), 4.28 (m, | |
| | 1-carboxylate | 1 H), 7.15 (d, 1 H), 12.28 (s, 1 H) | |

### Intermediate 17

### 3,4-Dichloro-N-1,4-dioxa-7-azaspiro[4.5]dec-10-yl-5-methyl-1H-pyrrole-2-carboxamide

Barium hydroxide (0.48 g, 2.8 mmol) was added to a suspension of methyl 10-{[(3,4-dichloro-5-methyl-1H-pyrrol-2-yl)carbonyl]amino}-1,4-dioxa-7-azaspiro[4.5]decane-7-carboxylate (0.55 g, 1.4 mmol, Intermediate 14) in methanol (8 mL) and water (2 mL). After heating in the microwave at 130° C. for two hours the reaction was cooled to room temperature and diluted with methanol. The suspension was filtered to remove insoluble barium salts. The filtrate was concentrated and the resulting residue was partitioned with EtOAc and water. The organic portion was dried with MgSO₄ and concentrated (0.40 g, 87%). MS (ES) (M+H)⁺: 334 for C₁₃H₁₇Cl₂N₃O₃; NMR: 1.48 (td, 1H), 1.81 (dd, 1H), 2.18 (s, 3H), 2.39 (d, 1H), 2.83 (d, 2H), 3.34 (s, 1H), 3.71-3.78 (m, 1H), 3.86-3.97 (m, 2H), 4.08-4.17 (m, 2H), 6.91 (d, 1H), 12.15 (s, 1H).

### Intermediates 18-19

The following Intermediates were prepared by the procedure described in Intermediate 17 from the starting materials (SM) indicated.

| Int | Compound | Data | SM |
|---|---|---|---|
| 18 | 3,4-Dichloro-N-1,5- | MS (ES) (M + H)⁺: 348 for C₁₄H₁₉Cl₂N₃O₃ | Intermediate |
| | dioxa-8- | NMR: 1.38-1.53 (m, 2 H), 1.70-1.85 (m, 2 | 15 |
| | azaspiro[5.5]undec-11- | H), 2.14-2.20 (m, 3 H), 2.23 (d, 1 H), | |
| | yl-5-methyl-1H-pyrrole- | 2.82 (d, 1 H), 3.27-3.42 (m, 2 H), 3.69 (s, 1 H), | |
| | 2-carboxamide | 3.84-3.98 (m, 3 H), 7.20 (d, 1 H) | |
| 19 | 3,4-Dichloro-N-(3,3- | MS (ES) (M − H)⁻: 392 for C₁₃H₁₉Cl₂N₃O₅; | Intermediate |
| | dimethoxypiperidin-4- | NMR: 1.67 (m, 1 H), 1.73 (m, 1 H), 2.27 (s, | 16 |
| | yl)-5-methyl-1H- | 3 H), 2.68 (m, 2 H), 2.76 (m, 2 H), 4.29 (m, | |
| | pyrrole-2-carboxamide | 1 H), 7.07 (d, 1 H), 12.22 (s, 1 H) | |

### Intermediate 20

### Ethyl 2-chloro-4-{[(2-methoxyethyl)amino]carbonyl}-1,3-thiazole-5-carboxylate

2,6-Lutidine (0.95 mL, 8.2 mmol) was added to a cold solution of ethyl 2-chloro-4-(chlorocarbonyl)-1,3-thiazole-5-carboxylate (2.08 g, 8.2 mmol) in anhydrous DCM (5 mL) followed by the dropwise addition of methoxyethylamine (0.71 mL, 8.2 mmol). The reaction was stirred at 0° C. for one hour and then overnight at room temperature. After removing solvent, the residue was partitioned with EtOAc and water, after which the organic portion was dried with MgSO₄ and concentrated to a brown oil (2.04 g, 85%). MS (ES) (M+H)⁺: 293 for C₁₀H₁₃ClN₂O₄S; NMR: 1.26 (t, 3H), 3.26 (s, 3H), 3.32-3.46 (m, 4H), 4.27 (q, 2H), 8.71 (s, 1H).

### Intermediate 21

### 3,4-Dichloro-N-[(3E)-3-(methoxyimino)piperidin-4-yl]-5-methyl-1H-pyrrole-2-carboxamide trifluoroacetate salt

A solution of 2.65 gm (6.3 mmol) of tert-butyl (3L)-4-{[(3,4-dichloro-5-methyl-1H-pyrrol-2-yl)carbonyl]amino}-3-(methoxyimino)piperidine-1-carboxylate (Intermediate 22) in 30 mL TFA and 30 ml dichloroethane was stirred at room temperature for 30 min. Solvent was removed to give a solid that was triturated with Et₂O and dried in vacuo to afford 2.5 gm of product. MS (ES) (M+H)⁺: 341 for C₁₂H₁₆Cl₂N₄O₂; NMR: 1.88 (m, 1H), 2.26 (s, 3H), 2.43 (m, 1H), 3.12-3.69 (m, 1H), 3.33 (m, 1H), 3.78 (d, 1H), 3.94 (s, 3H), 4.45 (d, 1H), 4.96 (m, 1H), 7.74 (d, 1H), 8.91 (s, broad, 2H), 12.23 (s, 1H). The product also contained some (<5%) of the corresponding (Z) isomer.

### Intermediate 22

### tert-Butyl (3E)-4-{[(3,4-dichloro-5-methyl-1H-pyrrol-2-yl)carbonyl]amino}-3-(methoxyimino)piperidine-1-carboxy late

A solution of 2.3 g (8.5 mmol) 3,4-dichloro-5-methyl-1H-pyrrole-2-carboxylic acid (Motekaitis, R. J.; Heinert, D. H.; Martell, Arthur E. J. Org. Chem. 35(8), 2504 (1970)), 2.3 g (8.5 mmol) tert-butyl (3E)-4-amino-3-(methoxyimino)piperidine-1-carboxylate (Intermediate 23), 1.13 g (8.5 mmol) HOBt, 3.2 g (16.2 mmol) EDC and 1.85 mL (16.2 mmol) NMM in 20 mL DCM was stirred at room temperature overnight. The mixture was diluted with EtOAc and washed with 1N HCl, water, aqueous Na₂CO₃, water and brine. Drying (MgSO₄) and removal of solvent gave a solid that was triturated with methanol to give 2.4 g of product as a white solid. MS (ES) (M+Na)⁺: 441 for C₁₇H₂₄Cl₂N₄O₄; NMR: 1.48 (s, 9H), 1.64 (m, 1H), 2.24 (s, 3H), 2.25 (m, 1H), 3.22 (m, 1H), 3.63-3.71 (d of m, 1H), 3.82 (s and m, 4H), 4.73 (m, 1H), 5.04 (d, 1H), 7.61 (d, 1H), 12.17 (s, 1H). The product also contained some (<5%) of the corresponding (Z) isomer.

### Intermediate 23

### tert-Butyl (3E)-4-amino-3-(methoxyimino)piperidine-1-carboxy late

A solution of 4.6 g (19 mmol) of tert-butyl (3E)-3-(methoxyimino)-4-oxopiperidine-1-carboxylate (Intermediate 24), 14.6 g (190 mmol) ammonium acetate and 1.2 g (19 mmol) NaBH₃CN in 50 ml MeOH was stirred at room temperature overnight. The mixture was acidified with 1N HCl, and methanol was removed to afford an aqueous residue that was extracted with EtOAc. The EtOAc was washed twice more with 1N HCl, and the combined aqueous washings were made basic with solid Na₂CO₃ before being extracted 3 times with DCM. The DCM extracts were washed with brine, dried (MgSO₄) and concentrated to give 2.3 g of product as an oil. NMR (CDCl₃): 1.56 (s, 9H), 1.64 (m, 3H), 2.23 (s, 3H), 2.69 (m, 1H), 3.28 (m, 1H), 3.67 (m, 1H), 3.97 (s, 3H), 4.74 (d, 1H), 5.01 (d, 1H), 7.61 (d, 1H), 12.18 (s, 1H). The product also contained some (<5%) of the corresponding (Z) isomer.

### Intermediate 24

### tert-Butyl (3E)-3-(methoxy imino)-4-oxopiperidine-1-carboxy late

A solution of 5.85 g (20.3 mmol) of tert-butyl (3E)-4,4-dimethoxy-3-(methoxyimino)piperidine-1-carboxylate (Intermediate 25) and 10 drops methanesulfonic acid in 40 mL acetone was heated at reflux for 40 min. The solution was treated with aqueous NaHCO₃ and extracted twice with EtOAc. The EtOAc washed with brine, dried (MgSO₄) and concentrated to give 4.6 g of product as an oil. NMR (CDCl₃): 1.59 (s, 9H), 2.79 (t, 2H), 3.82 (t, 2H), 4.18 (s, 3H), 4.53 (s, 2H). The product also contained some (<5%) of the corresponding (Z) isomer.

### Intermediate 25

### tert-Butyl (3E)-4,4-dimethoxy-3-(methoxyimino)piperidine-1-carboxylate

A solution of 5.0 g (19 mmol) of tert-butyl 4,4-dimethoxy-3-oxopiperidine-1-carboxylate (Intermediate 26), 2.4 g (29 mmol) methoxylamine hydrochloride and 2.8 g sodium acetate in 50 mL MeOH was heated at reflux for 2 hours. The solution was diluted with water and extracted 3 times with EtOAc. The EtOAc was washed with brine, dried (MgSO₄) and concentrated to give 5.85 g of product as an oil. NMR (CDCl₃): 1.45 (s, 9H), 3.25 (s, 6H), 3.67 (m, 2H), 4.02 (s, 3H), 4.32 (s, 2H). The product also contained some (<5%) of the corresponding (Z) isomer.

### Intermediate 26

### tert-Butyl 4,4-dimethoxy-3-oxopiperidine-1-carboxylate

Dry DMSO (3.2 mL, 45 mmol) was added dropwise to a solution of 11 mL (22 mmol) of 2N oxalyl chloride in DCM diluted with 30 mL DCM and cooled in a dry-ice acetone bath. After 5 min stirring, a solution of 5.0 g (19 mmol) of tert-butyl 3-hydroxy-4,4-dimethoxypiperidine-1-carboxylate (Intermediate 27) in 25 mL DCM was added dropwise. After stirring 20 min, 13 mL Et₃N was added, and the mixture was warmed to room temperature. The mixture was diluted with DCM and washed with water. The water layer was extracted 3 times more with DCM, and the combined organic extracts were washed with brine, dried (MgSO₄) and concentrated. The residue was taken up in Et₂O and insoluble solids were filtered and rinsed well with additional Et₂O. The filtrated was concentrated to give 5.0 g of product as an oil. NMR (CDCl₃): 1.45 (s, 9H), 2.11 (t, 2H), 3.33 (s, 6H), 3.78 (m, 2H), 4.12 (s, 2H).

### Intermediate 27

### tert-Butyl 3hydroxy-4,4-dimethoxypiperidine-1-carboxylate

A solution of 21.3 g (380 mmol) KOH in 100 mL dry MeOH was cooled in an ice water bath before 17.6 g (88 mmol) of tert-butyl 4-oxopiperidine-1-carboxylate was added portionwise. The mixture was stirred 20 min before 42.9 g (133 mmol) of iodobenzene diacetate was added portionwise and the mixture was allowed to warm to warm to room temperature with stirring overnight. Solvent was removed, and the residue was partitioned between water and EtOAc. The aqueous layer was saturated with NaCl and the EtOAc was separated. The aqueous layer was extracted 3 times more with EtOAc, and the combined EtOAc layers were dried (MgSO₄) and concentrated. Chromatography on silica gel (100% DCM with gradient elution to 100% EtOAc) afforded 21.3 g of product as a white solid. NMR (CDCl₃): 1.58 (s, 9H), 1.73-1.92 (m, 2H), 2.00 (s, broad, 1H), 2.91 (m, 1H), 2.05-2.34 (m, 2H), 3.25 (2s, 6H), 3.75 (m, 1H), 3.82-4.16 (m, 2H).

### Intermediate 28

### Allyl 4-(aminocarbonyl)-2-chloro-1,3-thiazole-5-carboxylate

A solution of 794 mg (2.2 mmol) allyl 2-chloro-4-{[(1-methyl-1-phenylethyl)amino]carbonyl}-1,3-thiazole-5-carboxylate (Intermediate 32) in 20 ml TFA was stirred at room temperature overnight. The mixture was diluted with 1,2-dichloroethane and solvent was removed. The residue was chromatographed on silica gel (100% DCM with gradient elution to 10% MeOH in DCM) to afford 350 mg of product. NMR: 4.84 (d, 2H), 5.33 (d, 1H), 5.42 (d, 1H), 5.88-6.13 (m, 1H), 7.91 (s, 1H), 8.12 (s, 1H).

### Intermediate 29

### Allyl 2-chloro-4-{[(2-methoxyethyl)amino]carbonyl}-1,3-thiazole-5-carboxylate

A solution of 0.5 g (1.6 mmol) of 5-[(allyloxy)carbonyl]-2-chloro-1,3-thiazole-4-carboxylic acid (Intermediate 33) in 20 mL SOCl₂ was heated at reflux for 20 min. Solvent was removed and the residue was diluted with DCM and solvent was again removed and the residual oil was placed in vacuo. The residue was dissolved in DCM and cooled in an ice water bath before 190 μL (1.6 mmol) 2,6-lutidine and 140 μL (1.6 mmol) 2-methoxyethylamine were added sequentially. The solution was warmed to room temperature and stirred overnight before being diluted with EtOAc and washed twice with aqueous Na₂CO₃, once with 1N HCl and once with brine. Drying (MgSO₄) and removal of solvent gave an oil that was chromatographed on silica gel (100% DCM with gradient elution to 40% EtOAc in DCM) to afford 245 mg of product as an oil. MS (ES) (M+Na)⁺: 329 for C₁₁H₁₃ClN₂O₄S; NMR: 3.48 (s, 3H), 3.55 (m, 2H), 3.77 (m, 2H), 4.86 (d, 2H), 5.33-5.52 (m, 2H), 5.88-6.16 (m, 1H), 7.81 (s, broad, 1H).

### Intermediates 30-32

The following Intermediates were prepared by the procedure described in Intermediate 29 using the reagents indicated.

| Int. | Compound | Data | Reagents |
|---|---|---|---|
| 30 | Methyl 2-chloro-4- | MS (ES) (M + H)⁺: 235 for | Intermediate |
| | [(methylamino)carbonyl]-1,3- | C₇H₇ClN₂O₃S; | 34 |
| | thiazole-5-carboxylate | NMR: 2.75 (d, 3 H), 3.84 (s, 3 H), | |
| | | 8.67 (m, 1 H) | |
| 31 | Isopropyl 2-chloro-4- | MS (ES) (M + H)⁺: 285 for | Intermediate |
| | [(methylamino)carbonyl]-1,3- | C₉H₁₁ClN₂O₃S; | 35 |
| | thiazole-5-carboxylate | NMR: 1.25 (d, 6 H), 2.75 (d, 3 H), | |
| | | 5.10 (septet, 1 H), 8.68 (m, 1 H) | |
| 32 | Allyl 2-chloro-4-{[(1-methyl-1- | MS (ES) (M + H)⁺: 365 for | Intermediate |
| | phenylethyl)amino]carbonyl}- | C₁₇H₁₇ClN₂O₃S | 33 |
| | 1,3-thiazole-5-carboxylate | | |

### Intermediate 33

### 5-[(Allyloxy)carbonyl]-2-chloro-1,3-thiazole-4-carboxylic acid

A solution of 3 mL H₂SO₄ and 12 mL water were cooled in an ice water bath, and 1.9 g (19 mmol) CrO₃ was added portionwise. The solution was added dropwise to a solution of 2.23 g (9.5 mmol) of allyl-2-chloro-4-(hydroxymethyl)-1,3-thiazole-5-carboxylate (Intermediate 36), and the mixture was stirred 3.5 hours with warming to room temperature. Isopropanol (2 mL) was added and stirring was continued for 10 min. The solution was diluted with water, saturated with NaCl and extracted 3 times with EtOAc. The EtOAc was washed with brine, dried (MgSO₄) and concentrated to give 2.2 g of an oil that slowly solidified. NMR: 5.43 (m, 2H), 6.01 (m, 1H), 14.21 (s, broad, 1H).

### Intermediates 34-35

The following Intermediates were prepared by the procedure described in Intermediate 33 using the reagents indicated.

| Int. | Compound | Data | Reagents |
|---|---|---|---|
| 34 | 2-Chloro-5-(methoxycarbonyl)- | MS (ES) (M − H)⁻: 220 for | Intermediate |
| | 1,3-thiazole-4-carboxylic acid | C₆H₄ClNO₄S; | 38 |
| | | NMR: 3.82 (s, 3 H), 14.14 (s, 1 H) | |
| 35 | 2-Chloro-5- | NMR: 1.32 (d, 6 H), 5.14 (m, 1 | Intermediate |
| | (isopropoxycarbonyl)-1,3- | H), 14.10 (s, 1 H) | 37 |
| | thiazole-4-carboxylic acid | | |

### Intermediate 36

### Allyl 2-chloro-4-(hydroxymethyl)-1,3-thiazole-5-carboxy late

t-Butylnitrite (4.2 mL, 31.5 mmol) was added slowly to a mixture of 4.5 g (21 mmol) of allyl 2-amino-4-(hydroxymethyl)-1,3-thiazole-5-carboxylate (Intermediate 40) and 4.2 g (31.5 mmol) CuCl₂ in 60 mL CH₃CN at room temperature. After stirring for 60 min, aqueous NaHSO₃ was added, and stirring was continued for 10 min. The mixture was partitioned between EtOAc and aqueous serine. The EtOAc was separated and washed with brine. The aqueous layer was extracted again with EtOAc, which was washed with brine. Drying (MgSO₄) of the combined EtOAc layers and removal of solvent gave an oil that was taken up in MeOH. Insoluble material was filtered off and rinsed with additional MeOH. The filtrate was concentrated and chromatographed on silica gel (100% DCM with gradient elution to 20% EtOAc in DCM) to afford 2.2 g of product as an oil. NMR (CDCl₃): 3.52 (t, 1H), 4.88 (d, 2H), 4.95 (d, 2H), 5.34-5.41 (m, 2H), 6.01 (m, 1H).

### Intermediate 37

### Isopropyl 2-chloro-4-(hydroxymethyl)-1,3-thiazole-5-carboxylate

Using the procedure of Intermediate 36, isopropy 2-amino-4-(hydroxymethyl)-1,3-thiazole-5-carboxylate (Intermediate 42) was converted to the title compound. NMR: 1.31 (d, 6H), 4.75 (d, 2H), 5.11 (septet, 1H), 5.43 (t, 1H).

### Intermediate 38

### Methyl 2-chloro-4-(hydroxymethyl)-1,3-thiazole-5-carboxylate

A mixture of 47 g (160 mmol) of methyl 2-amino-4-({[tert-butyl(dimethyl)silyl]oxy}methyl)-1,3-thiazole-5-carboxylate (Intermediate 39) and 32.8 g (230 mmol) CuCl₂ in 600 mL CH₃CN was cooled in an ice water bath before adding 29.9 mL (230 mmol) t-butylnitrite dropwise. After warming to room temperature and stirring 2 hours, solvent was removed and the residue was partitioned between 1N HCl and EtOAc with vigorous stirring overnight. The EtOAc was separated and washed with brine. The combined aqueous layers were saturated with NaCl and extracted 3 times with EtOAc, each extract being washed with additional brine. The combined EtOAc layers were dried (MgSO₄) and concentrated to afford an oil that slowly solidified in vacuo affording 31.6 g of product. NMR: 3.82 (s, 3H), 4.76 (s, 2H), 5.44 (s, 1H).

### Intermediate 39

### Methy 2-amino-4-({[tert-butyl(dimethyl)silyl]oxy}methyl)-1,3-thiazole-5-carboxylate

t-Butyldimethylsilyl chloride (26 g, 0.17 mol) was added to a solution of 30 g (0.16 mol) of methy 2-amino-4-(hydroxymethyl)-1,3-thiazole-5-carboxylate (Intermediate 41) and 21.4 g (0.31 mol) imidazole in 200 mL DMF. After stirring 2 hours, solvent was removed and the residue was taken up in 500 mL water. Solids were collected by filtration, washed well with additional water and dried in vacuo affording 47 g of product. MS (ES) (M+H)⁺: 303 for C₁₂H₂₂N₂O₃SSi; NMR: 0.03 (s, 6H), 0.92 (s, 9H), 3.74 (s, 3H), 4.86 (s, 2H), 7.83 (s, 2H).

### Intermediate 40

### Allyl 2-amino-4-(hydroxymethyl)-1,3-thiazole-5-carboxylate

A solution of 10 g (74 mmol) of 3-chlorofuran-2,4(3H,5H)-dione and 8.5 g (110 mmol) thiourea in 50 mL allyl alcohol was heated at 80° C. overnight. Solvent was removed and the residue was dissolved in water. The solution was treated with aqueous Na₂CO₃ precipitating solids. The solids were filtered, washed with water and dried in vacuo to afford 4.6 g of product as a white solid. NMR: 4.69 (d, 2H), 4.65 (d, 2H), 4.92 (t, 1H), 5.24-5.42 (m, 2H), 5.81-6.17 (m, 1H), 7.89 (s, 2H).

### Intermediates 41-42

The following Intermediates were prepared by the procedure described in Intermediate using the solvent indicated.

| Int. | Compound | Data | Solvent |
|---|---|---|---|
| 41 | Methyl 2-amino-4- | NMR: 3.73 (s, 3 H), 4.62 (d, 2 H), | methanol |
| | (hydroxymethyl)-1,3-thiazole- | 4.93 (t, 1 H), 7.57 (s, 2 H) | |
| | 5-carboxylate | | |
| 42 | Isopropyl 2-amino-4- | NMR: 1.24 (d, 6 H), 4.55 (d, 2 H), | isopropanol |
| | (hydroxymethyl)-1,3-thiazole- | 4.98 (t, 1 H), 5.0 (m, 1 H), 7.73 (s, 2 H) | |
| | 5-carboxylate | | |

### Intermediate 43

### Methyl 2-chloro-4-(1-methyl-1H-1,2,4-triazol-5-0)-1,3-thiazole-5-carboxylate

A solution of 3.9 g (56.5 mmol) NaNO₂ in 50 ml water was added dropwise to a solution of 4.64 g (19.6 mmol) of methyl 2-amino-4-(1-methyl-1H-1,2,4-triazol-5-yl)-1,3-thiazole-5-carboxylate (Intermediate 46) in 50 ml AcOH and 50 ml conc. HCl cooled in an ice water bath. After stirring 1 hour, 1.7 g urea dissolved in water was added dropwise. After stirring 1 hour, solvent was removed and the residue was portioned between EtOAc and aqueous NaHCO₃. The EtOAc was separated and washed with brine. Drying (MgSO₄) and removal of solvent gave 4.3 g of product as a solid. MS (ES) (M+H)⁺: 259 for C₈H₇ClNO₂S; NMR: 3.86 (s, 3H), 3.93 (s, 3H), 8.03 (s, 1H).

### Intermediate 44

The following Intermediate was prepared by the procedure described in Intermediate 43 using the reagent indicated.

| Int | Compound | Data | Reagent |
|---|---|---|---|
| 44 | Methyl 2-chloro-4-[1-(2- | MS (ES) (M + H)⁺: | Intermediate |
| | methoxyethyl)-1H-imidazol-2- | 302 for | 47 |
| | yl]-1,3-thiazole-5-carboxylate | C₁₁H₁₂ClN₃O₃S; | |
| | | NMR: 3.28 (s, 3 H), | |
| | | 3.61 (m, 2 H), | |
| | | 3.81 (s, 3 H), | |
| | | 4.20 (m, 2 H), | |
| | | 7.24 (s, 2 H). | |

### Intermediate 45

### Methyl 2-chloro-4-[1-(methoxymethyl)-1H-imidazol-2-yl]-1,3-thiazole-5-carboxylate

t-Butylnitrite (7.4 ml, 55.8 mmol) was added dropwise to a mixture of 10 g (37.3 mmol) methyl 2-amino-4-[1-(methoxymethyl)-1H-imidazol-2-yl]-1,3-thiazole-5-carboxylate (Intermediate 48) and 7.5 g CuCl₂ in 300 ml CH₃CN cooled in an ice water bath. After stirring 2 hours, solvent was removed and the residue was taken up in water and basified with aqueous Na₂CO₃. Serine (6 g) was added and the mixture was filtered through diatomaceous earth, rinsing through with water and MeOH. The MeOH was removed and the aqueous residue was saturated with NaCl and extracted 4 times with EtOAc. The EtOAc extracts were washed with brine, dried (MgSO₄) and concentrated to afford an oil that was chromatographed on silica gel (50% DCM in EtOAc followed by gradient elution to 100% EtOAc) to give product as a solid: NMR: 3.19 (s, 3H), 3.78 (s, 3H), 5.35 (s, 2H), 7.21 (s, 2 H).

### Intermediate 46

### Methyl 2-amino-4-(1-methyl-1H-1,2,4-triazol-5-yl)-1,3-thiazole-5-carboxylate

N-iodosuccinimide (6.6 g, 29 mmol) was added to a mixture of 5.33 g (29 mmol) methyl 3-(1-methyl-1H-1,2,4-triazol-5-yl)-3-oxopropanoate (Intermediate 49) and 5 g Amberlyst-15 resin in 50 ml EtOAc followed by stirring for 1 hour at room temperature. The resin was filtered off and rinsed with EtOAc. Solvent was removed from the filtrate and the residue was taken up in ethyl ether. Insoluble material was filtered off and rinsed with additional ether. Solvent was removed from the filtrate and the residue was dissolved in 60 ml MeOH before adding 3.44 g (45 mmol) thiourea. The mixture was heated at reflux for 1 hour. Solids precipitated on cooling. The solids were collected, washed with MeOH and dried in vacuo to give 2.1 g of product. Solvent was removed from the filtrate and the residue was taken up in water. Insoluble material was collected by filtration and rinsed well with water. The solids were dried in vacuo affording 2.1 g of additional product: MS (ES) (M+H)⁺: 240 for C₈H₉N₅O₂S; NMR: 3.62 (s, 3H), 3.67 (s, 3H), 7.03 (s, 1H), 8.13 (s, 2H).

### Intermediates 47-48

The following Intermediates were synthesized by an analogous method to Intermediate 46 from the starting materials (SM) given in the table below.

| Int | Compound | Data | SM |
|---|---|---|---|
| 47 | Methyl 2-amino-4-[1-(2- | MS (ES) (M + H)⁺: 283 for | Intermediate |
| | methoxyethyl)-1H-imidazol-2- | C₁₁H₁₄N₄O₃S; NMR: 3.18 (s, 3 H), | 50 |
| | yl]-1,3-thiazole-5-carboxylate | 3.62 (m, 2 H), 3.71 (s, 3 H), 4.28 (m, | |
| | | 2 H), 7.92 (s, 2 H), 8.41 (s, 2 H). | |
| 48 | Methyl 2-amino-4-[1- | MS (ES) (M + H)⁺: 269 for | Intermediate |
| | (methoxymethyl)-1H-imidazol- | C₁₀H₁₂N₄O₃S; NMR: 3.18 (s, 3 H), | 51 |
| | 2-yl]-1,3-thiazole-5- | 3.68 (s, 3 H), 5.52 (s, 2 H), 7.91 (s, 2 | |
| | carboxylate | H), 8.02 (s, 1 H), 8.41 (s, 1 H). | |

### Intermediate 49

### Methyl 3-(1-methyl-1H-1,2,4-triazol-5-yl)-3-oxopropanoate

NaH (7.84 g, 196 mmol of a 60% dispersion in oil) was added portionwise to a solution of 6.18 g (34.5 mmol) of 1-(1-methyl-1H-1,2,4-triazol-5-yl)ethanone (Ohta, S.; Kawasaki, I.; Fukuno, A.; Yamashita, M.; Tada, T.; Kawabata, T. Chem. Pharm. Bull. (1993), 41(7), 1226-31) in 100 ml dimethylcarbonate. The mixture was heated to 90° C. for 2 hour forming a thick slurry. After cooling to room temperature, the mixture was slowly transferred to 1N HCl over ice. The pH of the mixture was brought to about 7 with NaHCO₃ before being saturated with NaCl and extracted 4 times with EtOAc. The EtOAc was dried (MgSO₄) and concentrated to give an oil that was chromatographed on silica gel (100% DCM followed by gradient elution to 50% EtOAc in DCM). Product (5.3 g) was obtained as an oil. NMR: 3.77 (s, 3H), 4.10 (s, 2H), 4.22 (s, 3H), 7.88 (s, 1H).

### Intermediates 50-51

The following Intermediates were synthesized by an analogous method to Intermediate 49 from the starting materials (SM) given in the table below.

| Int | Compound | Data | SM |
|---|---|---|---|
| 50 | Methyl 3-[1-(2- | MS (ES) (M + H)⁺: 227 for C₁₀H₁₄N₂O_{4;} | Intermediate |
| | methoxyethyl)-1H- | NMR: 3.19 (s, 3 H), 3.62 (m, 5 H), | 52 |
| | imidazol-2-yl]-3- | 4.13 (s, 2 H), 4.48 (m, 2 H), 7.17 (s, 1 H), 7.58 (s, 1 H) | |
| | oxopropanoate | | |
| 51 | Methyl 3-[1-(2- | MS (ES) (M + H)⁺: 213 for C₁₀H₁₄N₂O_{4;} | Intermediate |
| | methoxymethyl)-1H- | NMR: 3.41 (s, 3 H), 3.75 (s, 3 H), | 53 |
| | imidazol-2-yl]-3- | 4.21 (s, 2 H), 5.75 (s, 2 H), 7.23 (s, 1 H), 7.34 (s, 1 H) | |
| | oxopropanoate | | |

### Intermediate 52

### 1-[1-(2-Methoxyethyl)-1H-imidazol-2-yl]ethanone

A solution of 28 ml (70 mmol) of 2.5 M n-butyllithium in hexanes was added slowly to a solution of 8.2 g (65 mmol) 1-(2-methoxyethyl)-1H-imidazole (WO 2003055876 A1) in 100 ml THF cooled in a dry ice-acetone bath. After stirring 1 hour, 7.5 ml (70 mmol) of N-methoxy-N-methylacetamide was added quickly, and the solution stirred for 40 min before being quenched with aqueous NH₄Cl. After warming to room temperature, the mixture was diluted with water and extracted 3 times with EtOAc, which was washed with brine, dried (MgSO₄) and concentrated to give 10.4 g of an oil that was used in subsequent steps without further purification. MS (ES) (M+H)⁺: 169 for C₈H₁₂CN₂O₂; NMR (CDCl₃): 2.71 (s, 3H), 3.31 (s, 3H), 3.72 (m, 2H), 4.62 (m, 2H), 7.14 (s, 1H), 7.22 (s, 1H).

### Intermediate 53

The following Intermediate was synthesized by an analogous method to Intermediate 52 from the starting materials (SM) given in the table below

| Int | Compound | Data | SM |
|---|---|---|---|
| 53 | 1-[1-(Methoxymethyl)- | NMR: 2.72 (s, 3 H), | 1-(methoxymethyl)-1H-imidazole |
| | 1H-imidazol-2- | 3.35 (s, 3 H), 5.75 (s, 2 | (Manoharn, T. S.; Brown, R. S. J. |
| | yl]ethanone | H), 7.21 (s, 1 H), | Org. Chem. (1989), 54(6), 1439-42). |
| | | 7.32 (s, 1 H). | |

### Intermediate 54

### Isopropyl 2-fluoroisonicotinate

A mixture of 2-fluoroisonicotinic acid and 16 ml trisopropylorthoformate in 100 ml toluene was heated at reflux for 2 h. The mixture as stirred with 1N HCl for 30 min before being partitioned between EtOAc and water. The EtOAc was separated, washed with water and brine, dried (MgSO₄) and concentrated. Chromatographed on silica gel (100% hexanes with gradient elution to 100% DCM to give product as an oil. NMR (CDCl₃): 1.43 (d, 6H), 5.39 (septet, 3H), 7.52 (m, 1H), 7.78 (m, 1H), 8.35 (d, 1H).

### Intermediate 55

### tert-Butyl 3-bromo-4-oxopiperidine-1-carboxylate

Chlorotrimethylsilane (5.6 ml, 44 mmol) was added slowly to a solution of tert-butyl 4-oxopiperidine-1-carboxylate (8 g, 40 mmol), triethylamine (12.3 ml, 88 mmol) and DMF (40 ml) at room temperature. The resultant solution was heated to 75° C. and stirred overnight under nitrogen. The reaction mixture was cooled to room temperature and then in an ice bath. Cold hexane (250 ml) was added slowly to the reaction mixture followed by cold (saturated) aqueous sodium bicarbonate (50 ml). The organic phase was separated and washed with brine, dried over sodium sulfate, filtered and concentrated under reduced pressure. The crude silyl enolether was dissolved in THF (15 ml) and cooled to 0° C. N-Bromosuccinimide (7.1 g, 40 mmol) was dissolved in THF (120 ml) and was added slowly (45 min.) to the reaction mixture. The resultant mixture was allowed to slowly warm to room temperature and stirred overnight. The reaction mixture was concentrated under reduced pressure. The crude residue was purified by flash chromatography (hexanes/EtOAc, 5:1) to provide the title compound as a white solid (11 g). MS (ESP): 222.1 (M-t-Bu) for C₁₀H₁₆BrNO₃; NMR: 1.25 (s, 9H), 2.30 (m, 1H), 2.55 (m, 1H), 3.42-3.80 (m, 3H), 3.93 (m, 1H), 4.60 (m, 1H).

### Intermediate 56

### tert-Butyl 3-(1H-imidazol-1-yl)-4-oxopiperidine-1-carboxylate

A solution of tert-butyl 3-bromo-4-oxopiperidine-1-carboxylate (Intermediate 55, 0.50 g, 1.8 mmol), imidazole (0.14 g, 2 mmol), potassium carbonate (0.25 g, 1.8 mmol) and DMF (3 ml) was warmed to 50° C. and stirred for 4 h. The reaction mixture was cooled to room temperature and diluted with EtOAc (50 ml). The resultant mixture was filtered and concentrated under reduced pressure. The crude residue was purified by flash chromatography (methanol/DCM gradient, 0-10%) to provide the title compound as a white solid (0.27 g). MS (ESP) (M−H)⁻: 264.2 for C₁₃H₁₉N₃O₃; NMR (CDCl₃): 1.41 (s, 9H), 2.50 (m, 2H), 2.55 (m, 1H), 3.14-3.33 (m, 2H), 4.50 (m, 1H), 4.79 (dd, 1H), 6.80 (s, 1H), 6.98 (s, 1H), 7.39 (s, 1 H).

### Intermediates 57-58

The following intermediates were prepared using the general method described for Intermediate 56 from tert-butyl 3-bromo-4-oxopiperidine-1-carboxylate (Intermediate 55) and the starting material (SM) indicated.

| Int | Compound | Data | SM |
|---|---|---|---|
| 57 | tert-Butyl 4-oxo-3- | MS (ESP) (M − H)⁻: 265 for C₁₂H₁₈N₄O₃ | 1,2,4-triazole |
| | (1H-1,2,4-triazol-1- | NMR (CDCl₃): 1.52 (s, 9 H), 2.67 (m, 2 H), | |
| | yl)piperidine-1- | 3.31 (m, 1 H), 3.67 (t, 1 H), 4.43 (m, 1 H), | |
| | carboxylate | 4.71 (m, 1 H), 5.05 (dd, 1 H), 8.01 (s, 1 H), | |
| | | 8.24 (s, 1 H) | |
| 58 | tert-Butyl 3-(3-chloro- | MS (ES) (M + H)⁺: 301 for C₁₂H₁₇ClN₄O₃ | 3-chloro- |
| | 1H-1,2,4-triazol-1-yl)- | NMR (CDCl₃): 1.49 (bs, 9 H), 2.64 (m, 2 H), | 1,2,4-triazole |
| | 4-oxopiperidine-1- | 3.26 (m, 1 H), 3.60 (t, 1 H), 4.40 (bs, 1 H), | |
| | carboxylate | 4.70 (m, 1 H), 4.93 (m, 1 H), 8.09 (s, 1 H) | |

### Intermediate 59

### cis(±)tert-Butyl 4-(benzylamino)-3-(1H-imidazol-1-yl)piperidine-1-carboxylate

Sodium triacetoxyborohydride (1.44 g, 6.80 mmol) was added in portions (one third of the total amount every hour over 2 h) to a stirred mixture of tent-butyl 3-(1H-imidazol-1-yl)-4-oxopiperidine-1-carboxylate (Intermediate 56, 1.2 g, 4.52 mmol), benzylamine (0.50 g, 4.75 mmol), and THF (10 ml) at room temperature. The resultant mixture was stirred for 18 h. The reaction was quenched by slow addition of water (5 ml) and the mixture was diluted with EtOAc (150 ml) and aqueous sodium bicarbonate (saturated solution). The organic layer was separated, washed with brine, dried over sodium sulfate, filtered and concentrated under reduced pressure. The crude residue was purified by flash chromatography (methanol/DCM gradient, 2-5%) to provide the title compound (0.89 g). MS (ESP) (M+H)⁺: 357 for C₂₀H₂₈N₄O₂; NMR (CDCl₃): 1.46 (s, 9H), 1.70 (m, 1H), 1.85 (m, 1H), 3.05 (m, 1H), 3.28 (m, 1H), 3.50 (m, 1H), 3.72 (ABq, 2H), 3.87 (m, 1H), 4.13 (m, 1H), 4.32 (m, 1H), 5.40 (br s, 1H), 7.10 (m, 2H), 7.20-7.36 (m, 5H), 7.76 (s, 1H).

### Intermediate 60

The following intermediate was prepared using the general method described for Intermediate 59 from benzylamine and the starting material (SM) indicated.

| Int | Compound | Data | SM |
|---|---|---|---|
| 60 | cis(±)tert-Butyl 4- | MS (ESP) (M + H)⁺: 358 for C₁₉H₂₇N₅O₂ | Intermediate |
| | (benzylamino)-3- | NMR (CDCl₃): 1.43 (s, 9 H), 1.78 (m, 3 H), | 57 |
| | (1H-1,2,4-triazol-1- | 3.14 (m, 1 H), 3.27 (m, 1 H), 3.55 (br d, 1 H), | |
| | yl)piperidine-1- | 3.76 (s, 2 H), 3.85 (m, 1 H), 4.24 (dd, 1 H), | |
| | carboxylate | 4.61 (m, 1 H), 7.21-7.33 (m, 5 H), 7.95 (s, 1 | |
| | | H), 8.24 (s, 1 H) | |

### Intermediate 61

### cis(±)tert-Butyl 4-amino-3-(1H-imidazol-1-yl)piperidine-1-carboxy late

A solution of cis(±)tert-butyl 4-(benzylamino)-3-(1H-imidazol-1-yl)piperidine-1-carboxylate (Intermediate 59, 0.89 g, 2.5 mmol), ammonium formate (0.63 g, 10 mmol), palladium on carbon (10%, 1.5 g, cat.) and methanol (12 ml) was warmed to 45° C. and stirred for 3 h (evolution of carbon dioxide). The mixture was warmed to 50° C. and stirred overnight. The reaction mixture was cooled to room temperature and filtered through diatomaceous earth to remove the palladium catalyst and the diatomaceous earth pad was washed with methanol (50 ml). The filtrate was concentrated under reduced pressure to provide the crude amine (0.71 g). The amine was used in the next step without further purification. MS (ESP) (M+H)⁺: 267 for C₁₃H₂₂N₄O_{2l.}

### Intermediate 62

The following intermediate was prepared using the general method described for Intermediate 61 from the starting material (SM) indicated.

| Int | Compound | Data | SM |
|---|---|---|---|
| 62 | cis(±)tert-Butyl 4-amino-3- | MS (ESP) (M + H)⁺: | Intermediate |
| | (1H-1,2,4-triazol-1- | 268 for | 60 |
| | yl)piperidine-1- | C₁₂H₂₁N₅O₂ | |
| | carboxylate dihydrochloride | | |

### Intermediate 63

### cis(±)tert-Butyl 4-amino-3-(3-chloro-1H-1,2,4-triazol-1-yl)piperidine-1-carboxylate

To a stirred solution of tert-butyl 3-(3-chloro-1H-1,2,4-triazol-1-yl)-4-oxopiperidine-1-carboxylate (Intermediate 58, 1.28 g, 4.2 mmol) and ammonium trifluoroacetate (5.5 g, 42 mmol) in THF (10 mL), at room temperature under N₂, was added sodium triacetoxyborohydride (1.35 g, 6.4 mmol) as a solid, in two portions over twenty minutes. The mixture was stirred under these conditions for approx. 2 hours. Complete conversion was indicated after this time by thin layer chromatography (1:1 EtOAc:hexane). The reaction mixture was poured into an aqueous solution of sodium bicarbonate (approx. 20 mL) and diluted with 35 mL EtOAc. The mixture was shaken, and the organic layer was separated. The aqueous layer was back-extracted twice with 45-mL portions of 3.5:1 THF:EtOAc. The organic layers were combined, dried over sodium sulfate, filtered, and concentrated under reduced pressure. The resulting off-white solid was taken forward without purification. MS (ES) (M+H)⁺: 302 for C₁₂H₂₀ClN₅O₂.

### Intermediate 64

### cis(±)tert-Butyl-4-{[(3,4-dichloro-5-methyl-1H-pyrrol-2-yl)carbonyl]amino}-3-(1H-imidazol-1-yl)piperidine-1-carboxylate

To a stirred solution of 3,4-dichloro-5-methyl-1H-pyrrole-2-carboxylic acid (described previously in WO 2005026149 A1, 0.434 g, 2.24 mmol) in DMF (5 mL) was added triethylamine (0.69 ml, 4.90 mmol) followed by the addition of HATU (0.851 g, 2.24 mmol) at room temperature. The resultant solution was stirred for 0.5 h and cis(±)tert-butyl 4-amino-3-(1H-imidazol-1-yl)piperidine-1-carboxylate (Intermediate 61, 0.700 g, 2.24 mmol) was added to the mixture. The reaction was allowed to stir overnight. The reaction mixture was concentrated and the residue was partitioned between water and EtOAc. The layers were separated and the aqueous layer was extracted with EtOAc two more times. The organic extracts were combined and washed with saturated NaHCO₃ solution followed by water and brine. It was dried over sodium sulfate and concentrated under reduced pressure. The crude residue was purified by flash chromatography (methanol/DCM gradient, 2-10%) to provide the title compound (0.70 g). MS (ESP) (M+H)⁺: 442 for C₁₉H₂₅Cl₂N₅O₃; NMR: 1.40 (br. s, 9H), 1.80 (m, 2H), 2.17 (s, 3H), 3.10 (m, 1H), 3.66-3.90 (m, 2H), 3.96 (m, 1H), 4.50 (m, 1H), 4.70 (m, 1H), 6.97 (d, 1H), 7.13 (s, 1H), 7.24 (s, 1H), 7.97 (s, 1H), 12.01 (s, 1H).

### Intermediates 65-66

The following intermediates were prepared using the general method described for Intermediate 64 from 3,4-dichloro-5-methyl-1H-pyrrole-2-carboxylic acid and the starting material (SM) indicated.

| Int | Compound | Data | SM |
|---|---|---|---|
| 65 | cis(±)tert-Butyl 4- | MS (ESP) (M + H)⁺: 443 for C₁₈H₂₄Cl₂N₆O₃ | Intermediate |
| | {[(3,4-dichloro-5- | NMR: 1.16-1.40 (br s, 9 H), 1.74 (m, 1 H), | 62 |
| | methyl-1H-pyrrol-2- | 2.00-2.20 (m, 1 H), 2.15 (s, 3 H), 3.09 (m, 1 | |
| | yl)carbonyl]amino}-3- | H), 3.55 (d, 1 H), 3.95 (m, 1 H), 4.16 (m, 1 | |
| | (1H-1,2,4-triazol-1- | H), 4.59 (m, 1 H), 4.82 (br s, 1 H), 7.16 (d, 1 | |
| | yl)piperidine-1- | H), 8.02 (s, 1 H), 8.51 (s, 1 H), 12.08 (s, 1 | |
| | carboxylate | H) | |
| 66 | cis(±)tert-Butyl 3-(3- | MS (ES) (M + H)⁺: 476 for C₁₉H₂₄Cl₃N₅O₃ | Intermediate |
| | chloro-1H-1,2,4- | NMR: 1.25 (br s, 9 H), 1.72 (m, 2 H), | 63 |
| | triazol-1-yl)-4-{[(3,4- | 2.14 (m, 2 H), 3.07 (br s, 2 H), 3.54 (m, 1 H), | |
| | dichloro-5-methyl- | 4.02 (m, 1 H), 4.14 (m, 2 H), 4.55 (m, 1 H), | |
| | 1H-pyrrol-2- | 4.80 (m, 1 H), 7.11 (br s, 1 H), 8.53 (s, 1 H), | |
| | yl)carbonyl]amino}piperidine- | 12.05 (s, 1 H) | |
| | 1- | | |
| | carboxylate | | |

### Intermediate 67

### cis(±)3,4-Dichloro-N-[3-(1H-imidazol-1-yl)piperidin-4-yl]-5-methyl-1H-pyrrole-2-carboxamide dihydrochloride

A solution of 4 M hydrochloric acid in dioxane (4 ml, 16 mmol) was added to a solution of cis(±)tert-butyl-4-{[(3,4-dichloro-5-methyl-1H-pyrrol-2-yl)carbonyl]amino}-3-(1H-imidazol-1-yl)piperidine-1-carboxylate (Intermediate 64, 0.697 g, 1.58 mmol) in dioxane (1 ml) at room temperature. The mixture was stirred for 4 h and then concentrated under reduced pressure to yield the crude amine salt (600 mg). The amine was used in the next step without further purification. MS (ESP) (M+H)⁺: 342 for C₁₄H₇Cl₂N₅O.

### Intermediates 68-69

The following intermediates were prepared using the general method described for Intermediate 67 from the starting material (SM) indicated.

| Int | Compound | Data | SM |
|---|---|---|---|
| 68 | cis(±)3,4-Dichloro-5-methyl-N-[3- | MS (ESP) (M + H)⁺: 343 for | Intermediate |
| | (1H-1,2,4-triazol-1-yl)piperidin-4- | C₁₃H₁₆Cl₂N₆O | 65 |
| | yl]-1H-pyrrole-2-carboxamide | | |
| | dihydrochloride | | |
| 69 | cis(±)3,4-Dichloro-N-[3-(3-chloro- | MS (ES) (M + H)⁺: 377 for | Intermediate |
| | 1H-1,2,4-triazol-1-yl)piperidine-4- | C₁₃H₁₅Cl₃N₆O | 66 |
| | yl]-5-methyl-1H-pyrrole-2- | NMR: 2.05 (br s, 2 H), 2.17 (s, | |
| | carboxamide dihydrochloride | 3 H), 3.22 (m, 1 H), 3.47 (m, 1 | |
| | | H), 3.62 (m, 1 H), 3.91 (m, 1 | |
| | | H), 4.71 (br s, 1 H), 5.07 (m, 1 | |
| | | H), 7.33 (br s, 2 H), 7.88 (d, 1 | |
| | | H), 8.62 (s, 1 H), 8.85 (br s, 1 | |
| | | H), 9.71 (br s, 1 H), 12.52 (s, 1 | |
| | | H) | |

### Intermediate 70

trans(±)tert-Butyl-4-[(diphenylmethylene)amino]-3-hydroxypiperidine-1-carboxylate

trans(±)tert-Butyl-4-amino-3-hydroxypiperidine-1-carboxylate (Tetrahedron Lett, 2002, 4289, 11.9 g; 55 mmol) and benzophenone imine (10 g; 59 mmol; 1.05 eq.) were dissolved in anhydrous toluene and heated to reflux for 18 hrs. Monitored the reaction by TLC (30% EtOAc/hexanes with 0.1% triethylamine). The crude reaction was concentrated and purified by flash column chromatography. Isolation gave 18.4 g of the title compound in an 86% yield. LC/MS (ES⁺) (M+H)⁺: 381 for C₂₃H₂₈N₂O₃.

### Intermediate 71

### cis(±)tert-Butyl-3-azido-4-[(diphenylmethylene)amino]piperidine-1-carboxylate

In a flame-dried flask triphenylphosphine (3.86 g; 14.7 mmol; 2 eq.) was dissolved in anhydrous THF (15 ml) and cooled to 0° C. DIAD (2.97 g; 14.7 mmol; 2 eq.) was slowly added dropwise. Upon addition a white precipitate formed. A THF solution containing trans(±)tert-butyl-4-[(diphenylmethylene)amino]-3-hydroxypiperidine-1-carboxylate (Intermediate 70, 2.8 g; 7.36 mmol) was added (amount of THF added was such that the final concentration of alcohol was ca. 0.5-1M). The resultant reaction slurry was stirred at 0° C. for 30 minutes. (PhO)₂PON₃ (4.05 g; 14.7 mmol; 2 eq.) was then added and the reaction was allowed to warm to RT and stirred for 12 hrs. Monitored by LC/MS. The reaction was concentrated and purified by flash column chromatography (0-30% EtOAc/hexanes with 0.1% triethylamine). Isolation gave 2.13 g of the title compound in 71% yield. LC/MS (ES) (M+H)⁺: 406 for C₂₃H₂₇N₅O₂.

### Intermediate 72

### cis(±)tert-Butyl-3-azido-4-{[(3,4-dichloro-5-methyl-1H-pyrrol-2-yl)carbonyl]amino}piperidine-1-carboxylate

cis(±)tert-Butyl-3-azido-4-[(diphenylmethylene)amino]piperidine-1-carboxylate (Intermediate 71, 1.36 g; 3.3 mmol) was dissolved in 10 ml of aqueous THF (5% H₂O). Pyridinium p-toluene sulphonate (850 mg; 3.4 mmol; 1.03 eq.) was added in a single portion. The initial cloudy solution became clear within minutes. Upon completion (as determined by LC/MS analysis) the reaction was concentrated and azeotropically dried with acetonitrile. No further purification.

Crude cis(±)tert-butyl-4-amino-3-azidopiperidine-1-carboxylate (3.3 mmol) was dissolved in anhydrous DCM (10 ml) and DIEA (1.27 g; 1.6 ml; 9.9 mmol; 3 eq.). The solution was cooled to 0° C. and 3,4-dichloro-5-methyl-1H-pyrrole-2-carbonyl chloride (736 mg; 3.5 mmol; 1.05 eq.) was added. The reaction was complete within 30 minutes. Dilute with DCM and wash with H₂O (×2), brine and dried over Na₂SO₄. Filter and concentrate. Purify by flash column chromatography (0-60% EtOAc/hexanes). Isolation gave 967 mg in 69% yield over the two-step sequence. LC/MS (ES⁻)[(M−H)⁻]: 415, 417 for C₁₆H₂₂Cl₂N₆O₃.

### Intermediate 73

### cis(±)N-(3-Azidopiperidin-4-yl)-3,4-dichloro-5-methyl-1H-pyrrole-2-carboxamide hydrochloride

cis(±)tert-Butyl-3-azido-4-{[(3,4-dichloro-5-methyl-1H-pyrrol-2-yl)carbonyl]amino}piperidine-1-carboxylate (Intermediate 72, 967 mg; 2.3 mmol) was dissolved in 4N HCl in dioxanes (20 ml) and methanol (10 ml). The solution was stirred for 2 hours and monitored by LC/MS. Upon completion the solvent was removed and the crude reaction mixture was azeotroped with methanol to remove excess HCl. No further purification. LC/MS (ES⁺) (M+H)⁺: 317, 319 for C₁₁H₁₄Cl₂N₆O.

### Intermediate 74 and Intermediate 75

### Benzyl (3R,4R)-4-amino-3-hydroxypiperidine-1-carboxylate and Benzyl (3S,4S)-4-amino-3-hydroxypiperidine-1-carboxylate

trans(±)Benzyl-4-azido-3-hydroxypiperidine-1-carboxylate (Tetrahedron Lett., 2002, 4289-4293, 23 grams; 83.2 mmol) and triphenyl phosphine (24 grams; 91.5 mmol; 1.1 equiv.) were combined and dissolved in anhydrous THF (100 ml). The reaction solution was stirred at room temperature for 2 hours. A white precipitate formed upon stirring. Monitored the progress of the reaction by TLC—loss of starting material (1:1 EtOAc/Hex). The THF was removed by rotary evaporation. The crude reaction mixture was dissolved in methanol (100 ml) and 0.5 N NaOH (100 ml). The suspension was stirred at room temperature overnight. Monitored the hydrolysis by TLC (90:10:1 CHCl₃/MeOH/NH₄O H). The methanol was removed by rotary evaporation. Diluted the crude reaction mixture with water and extracted with chloroform. The organic layer was dried over Na₂SO₄, filtered and concentrated. Purified by flash column chromatography. Isolation gave 15.5 grams of the title compound in a 75% yield. LC/MS (ES⁺) (M+H)⁺: 251 for C₁₃H₁₈N₂O₃.

The enantiomers were separated by chiral column chromatography. Column: AD 250×4.6 mm, 10 microns. Eluent: 50% hexanes, 50% 1:1 ethanol-methanol, 0.1% diethyl amine. Flow: 1 ml/min. Separation gave 6.5 grams of benzyl (3R,4R)-4-amino-3-hydroxypiperidine-1-carboxylate and 6.8 grams of benzyl (3S,45)-4-amino-3-hydroxypiperidine-1-carboxylate. LC/MS (ES) (M+H)⁺: 251 for C₁₃H₁₈N₂O₃; NMR (d₄-MeOD): 1.35 (m, 1H), 1.82 (m, 1H), 2.64 (m, 2H), 2.83 (m, 1H), 3.11 (m, 1H), 4.00 (m, 2H), 5.12 (s, 2H), 7.34 (m, 5H).

### Intermediate 76

### Benzyl(3R,4R)-4-[(diphenylmethylene)amino]-3-hydroxypiperidine-1-carboxylate

Benzyl (3R,4R)-4-amino-3-hydroxypiperidine-1-carboxylate (Intermediate 74, 6.5 grams; 25.9 mmol) and benzophenone imine 5.1 grams; 27.3 mmol; 1.05 equiv.) were combined and dissolved in anhydrous toluene (50 ml). Heated to reflux for 12 hours. Monitored the reaction by TLC (30% EtOAc/Hex). Concentrated the reaction to an oil and purified by flash column chromatography (10%-40% EtOAc/hex with 0.1% DIEA). Isolation gave 10 grams of the title compound as a white foam. LC/MS (ES) (M+H)⁺: 415 for C₂₆H₂₆N₂O₃; NMR (CDCl₃): 1.54 (m, 1H), 1.73 (m, 1H), 2.26 (m, 1H), 2.85 (m, 2H), 3.35 (m, 1H), 3.86 (m, 1H), 4.03 (m, 1H), 4.24 (m, 1H), 5.12 (s, 2H), 7.18-7.68 (m, 15H).

### Intermediate 77

### Benzyl (3S,4R)-3-azido-4-[(diphenylmethylene)amino]piperidine-1-carboxylate

Triphenyl phosphine (3.5 grams; 13.5 mmol; 2 equiv.) was dissolved in anhydrous THF (25 ml). The solution was cooled to 0° C. A THF solution containing diisopropyldiazodicarboxylate (2.7 grams; 13.5 mmol; 2 equiv.) was added dropwise. A white precipitate formed upon addition. After stirring for 30 minutes at 0° C., benzyl (3R,4R)-4-[(diphenylmethylene)amino]-3-hydroxypiperidine-1-carboxylate (Intermediate 76, 2.8 grams; 6.7 mmol) was added in a single portion. Stirred for an additional 2 hours. Diphenylphosphoryl azide (3.7 grams; 13.5 mmol; 2 equiv.) was then added dropwise. The resultant slurry was warmed to room temperature and stirred for 12 hours. Monitored the reaction by TLC (30% EtOAc/Hex) and LC/MS. Concentrated the reaction to an oil and purified by flash column chromatography. Isolation gave 1.28 grams of the title compound in a 50% yield. LC/MS (ES⁺) (M+H)⁺: 440 for C₂₆H₂₅N₅O₂; NMR (CDCl₃): 1.52 (m, 1H), 1.91 (m, 1H), 3.34 (m, 1H), 3.54 (m, 2H), 3.85 (m, 2H), 4.06 (m, 1H), 5.17 (s, 2H), 7.25-7.72 (m, 15H).

### Intermediate 78

### Benzyl (3S,4R)-4-amino-3-azidopiperidine-1-carboxylate

Benzyl (3S,4R)-3-azido-4-[(diphenylmethylene)amino]piperidine-1-carboxylate (Intermediate 77, 1.28 grams; 2.9 mmol) and PPTS (804 mg; 3.2 mmol; 1.1 equiv.) were combined in THF and water. The reaction was stirred at room temperature for 12 hours. Monitored by TLC (30% EtOAc/Hex)—loss of starting material and formation of benzophenone. The reaction was concentrated and azeotropically dried with acetonitrile. No further purification. LC/MS (ES) (M+H)⁺: 276 for C₁₃H₁₇N₅O₂.

### Intermediate 79

### Benzyl (3S,4R)-3-azido-4-{[(3,4-dichloro-5-methyl-1H-pyrrol-2-yl)carbonyl]amino}piperidine-1-carboxylate

3,4-Dichloro-5-methyl-1H-pyrrole-2-carboxylic acid (622 mg; 3.2 mmol; 1.1 equiv.) and HATU (1.44 grams; 3.8 mmol; 1.3 equiv.) were dissolved in DMF (10 ml) and DIEA (1.1 grams; 8.7 mmol; 3 equiv.). The solution was stirred for 30 minutes. The crude benzyl (3S,4R)-4-amino-3-azidopiperidine-1-carboxylate (Intermediate 78, 2.9 mmol) was then added in a single portion. Monitored the reaction by LC/MS and TLC (50% EtOAc/Hex)-ninhydrin stain. Diluted the reaction with EtOAc and washed with water (×2), then brine. Dried over Na₂SO₄, filtered and concentrated to an oil. The compound was purified by flash column chromatography (20%-50% EtOAc/Hex). Isolation gave 914 mg of the title compound in a 70% yield. LC/MS (ES) (M+H)⁺: 451, 453 for C₁₉H₂₀Cl₂N₆O₃; NMR: 1.64 (m, 2H), 2.23 (s, 3H), 2.95 (m, 1H), 3.23 (m, 1H), 4.00 (m, 4H), 5.14 (s, 2H), 7.18 (d, 1H), 7.31 (m, 5H), 12.12 (s, 1H).

### Intermediate 80

### Benzyl (3S,4R)-4-{[(3,4-dichloro-5-methyl-1H-pyrrol-2-yl)carbonyl]amino}-3-(1H-1,2,3-triazol-1-yl)piperidine-1-carboxy late

Benzyl (3S,4R)-3-azido-4-{[(3,4-dichloro-5-methyl-1H-pyrrol-2yl)carbonyl]amino}piperidine-1-carboxylate (Intermediate 79, 300 mg; 0.66 mmol) was dissolved in NMP (1 ml) and 1,4 dioxane (1 ml). Bicyclo[2.2.1]hepta-2,5-diene (2 ml) was then added and the resultant reaction solution was heated in the microwave at 120° C. for 30 minutes. Monitored by LC/MS. Diluted with EtOAc and washed with water (×2). Dried the organic layer over Na₂SO₄, filtered and concentrated the reaction mixture to an oil. The crude mixture was clean by LC/MS and NMR. No further purification. LC/MS (ES) (M+H)⁺: 477, 479 for C₂₁H₂₂Cl₂N₆O₃.

### Intermediate 81

### Benzyl (3S,4R)-3-(4-chloro-1H-1,2,3-triazol-1-yl)-4-{[(3,4-dichloro-5-methyl-1H-pyrrol-2-yl)carbonyl]amino}piperidine-1-carboxylate

Benzyl (3S,4R)-3-azido-4-{[(3,4-dichloro-5-methyl-1H-pyrrol-2yl)carbonyl]amino}piperidine-1-carboxylate (Intermediate 79, 300 mg; 0.66 mmol) was dissolved in 1,4 dioxane (3 ml). 1-Chloroethylenesulfonyl chloride (500 mg; 3.1 mmol; 4.7 equiv.) was then added and the resultant reaction solution was heated to 90° C. for 12 hours. Monitored by LC/MS. The dioxane was removed by rotary evaporation and the resultant reaction mixture was taken up in EtOAc. Washed with water (×2). Dried the organic layer over Na₂SO₄, filtered and concentrated to a solid. Purified by flash column chromatography (50%-100% EtOAc/Hex). LC/MS (ES⁺) (M+H)⁺: 511, 513 for C₂₁H₂₁Cl₃N₆O₃; NMR: 1.82 (m, 1H), 2.16 (s, 3H), 2.19 (m, 1H), 3.27 (m, 1H), 3.76 (m, 1H), 4.07 (m, 1H), 4.18 (m, 1H), 4.65 (m, 1H), 4.93 (m, 3H), 7.19 (d, 1H), 7.34 (m, 5H), 8.34 (s, 1H), 12.02 (s, 1H).

### Intermediate 82

### Benzyl (3S,4R)-3-(4-bromo-1H-1,2,3-triazol-1-yl)-4-{[(3,4-dichloro-5-methyl-1H-pyrrol-2-yl)carbonyl]amino}piperidine-1-carboxylate

Benzyl(3S,4R)-3-azido-4-{[(3,4-dichloro-5-methyl-1H-pyrrol-2yl)carbonyl]amino}piperidine-1-carboxylate (Intermediate 79, 200 mg; 0.44 mmol) was dissolved in 1,4 dioxane (4 ml). 1-Bromoethylenesulfonyl chloride (364 mg; 1.8 mmol; 4 equiv.) was then added and the resultant reaction solution was heated to 90° C. for 12 hours. Monitored by LC/MS. The dioxane was removed by rotary evaporation and the resultant reaction mixture was taken up in EtOAc. Washed with 10% NaHCO₃. Dried the organic layer over Na₂SO₄, filtered and concentrated to a solid. Purified by flash column chromatography (10%-100% EtOAc/Hex). Isolation gave 200 mg's of the title compound in 81% yield. LC/MS (ES⁺) (M+H)⁺: 555, 557, 559 for C₂₁H₂₁BrCl₂N₆O₃.

### Intermediate 83

### Benzyl (3S,4R)-3-(4-cyano-1H-1,2,3-triazol-1-yl)-4-{[(3,4-dichloro-5-methyl-1H-pyrrol-2yl)carbonyl]amino}piperidine-1-carboxylate

Benzyl (3S,4R)-3-azido-4-{[(3,4-dichloro-5-methyl-1H-pyrrol-2yl)carbonyl]amino}piperidine-1-carboxylate (Intermediate 79, 300 mg; 0.66 mmol) was dissolved in chloroacrylonitrile (3 ml) and acetonitrile (3 ml). The resultant reaction solution was heated to 90° C. for 48 hours. Monitored by LC/MS. The acetonitrile was removed by rotary evaporation and the resultant reaction mixture was taken up in EtOAc. Washed with water (×2). Dried the organic layer over Na₂SO₄, filtered and concentrated to a solid. Purified by flash column chromatography (50%-100% EtOAc/Hex). LC/MS (ES) (M+H)⁺: 502, 504 for C₂₂H₂₁Cl₂N₇O₃; NMR (CDCl₃): 2.05 (m, 2H), 2.34 (s, 3H), 3.29 (m, 1H), 3.69 (m, 1H), 4.28 (m, 1H), 4.74 (m, 2H), 5.13 (m, 3H), 7.28 (m, 6H), 8.21 (m, 1H), 9.90 (m, 1H).

### Intermediate 84

### tert-Butyl (3S,4R)-3-amino-4-{[(3,4-dichloro-5-methyl-1H-pyrrol-2-yl)carbonyl]amino}piperidine-1-carboxylate

(3S,4R)-tent-Butyl-3-azido-4-{[(3,4-dichloro-5-methyl-1H-pyrrol-2-yl)carbonyl]amino}piperidine-1-carboxylate (Intermediate 103, 300 mg; 0.72 mmol) and triphenyl phosphine (208 mg; 0.79 mmol; 1.1 equiv.) were combined and dissolved in THF (3 ml). The solution was stirred at room temperature overnight. Monitored the loss of starting material by LC/MS and TLC (70% EtOAc/Hex). The solvent was removed by rotary evaporation and dissolved in methanol (5 ml) and 0.5 N NaOH (2 ml). The slurry was warmed gently to obtain a homogeneous solution. Stir overnight. The methanol was removed under vacuum. The crude reaction mixture was diluted with water and extracted with chloroform. The organic phase was dried over Na₂SO₄, filtered and concentrated. No further purification. LC/MS (ES) (M+H)⁺: 391, 393 for C₁₆H₂₄Cl₂N₄O₃.

### Intermediate 85

### tert-Butyl (3S,4R)-4-{[(3,4-dichloro-5-methyl-1H-pyrrol-2-yl)carbonyl]amino}-3-(4-methyl-1H-1,2,3-triazol-1-yl)piperidine-1-carboxylate

tert-Butyl (3S,4R)-3-amino-4-{[(3,4-dichloro-5-methyl-1H-pyrrol-2-yl)carbonyl]amino}piperidine-1-carboxylate (Intermediate 84, 0.72 mmol) was dissolved in chloroform and DIEA (272 mg; 2.16 mmol; 3 equiv.). N-[(1E)-2,2-Dichloro-1-methylethylidene]-4-methylbenzenesulfonohydrazide (234 mg; 0.79 mmol; 1.1 equiv.) was added in a single portion. Monitored the reaction by LC/MS. Reaction was complete in 2 hours. Diluted with chloroform, washed with water and dried organic layer over Na₂SO₄. The product was purified by flash column chromatography (0-100% EtOAc/Hex). Isolation gave 258 mg of the title compound in a 78% yield. LC/MS (ES) (M+H)⁺: 457, 459 for C₁₉H₂₆Cl₂N₆O₃.

### Intermediate 86

### Benzyl (3S,4R)-4-{[(3,4-dichloro-5-methyl-1H-pyrrol-2-yl)carbonyl]amino}-3-[4-(hydroxymethyl)-1H-1,2,3-triazol-1-yl]piperidine-1-carboxylate

Benzyl (3S,4R)-3-azido-4-{[(3,4-dichloro-5-methyl-1H-pyrrol-2-yl)carbonyl]amino}piperidine-1-carboxylate (Intermediate 79, 1 mmol) was dissolved in a 1:1 mixture of t-butanol and water (7 ml total). Propargyl alcohol (450 mg; 1 mmol; 1 equiv.) and sodium ascorbate (110 mg; 2 mmol; 2 equiv.) were added in a single portion. Copper sulfate (5.4 mg) was then added and the resultant reaction mixture was stirred at room temperature for 18 hours. Monitored the reaction by LC/MS and TLC (9:1 EtOAc/hex). The solvent was removed by rotary evaporation and diluted with EtOAc. Washed with sat. NH₄Cl, brine, dried over Na₂SO₄ and filtered. The crude reaction mixture was concentrated and purified by flash column chromatography (20-100% EtOAc/Hex). Isolation gave 370 mg of the title compound in 73% yield. LC/MS (ES⁺) (M+H)⁺: 507, 509 for C₂₂H₂₄Cl₂N₆O₄.

### Intermediate 87

### Benzyl (3S,4R)-4-{[(3,4-dichloro-5-methyl-1H-pyrrol-2-yl)carbonyl]amino}-3-(4-{[(diphenoxyphosphoryl)oxy]methyl}-1H-1,2,3-triazol-1-yl)piperidine-1-carboxylate

Benzyl (3S,4R)-4-{[(3,4-dichloro-5-methyl-1H-pyrrol-2-yl)carbonyl]amino}-3-[4-(hydroxymethyl)-1H-1,2,3-triazol-1-yl]piperidine-1-carboxylate (Intermediate 86, 100 mg; 0.2 mmol) was dissolved in a 2:1 mixture of DCM/pyridine (1 ml). A DCM solution (1 ml) containing diphenyl phosphorochloridate (0.04 ml) was added at 0° C. The reaction was stirred at 5° C. for 4 hours and then quenched with phosphate buffer (pH=7). Diluted with EtOAc, washed with water and dried over Na₂SO₄. Filtered and concentrated to an oil. Purified by flash column chromatography 70% EtOAc/hexanes. Isolation gave 80 mg of the title compound in 55% yield. LC/MS (ES⁺) (M+H)⁺: 739, 741 for C₃₄H₃₃Cl₂N₆O₇P.

### Intermediate 88

### Benzyl (3S,4R)-3-[4-(cyanomethyl)-1H-1,2,3-triazol-1-yl]-4-{[(3,4-dichloro-5-methyl-1H-pyrrol-2-yl)carbonyl]amino}piperidine-1-carboxylate

Benzyl (3S,4R)-4-{[(3,4-dichloro-5-methyl-1H-pyrrol-2-yl)carbonyl]amino}-3-(4-{[(diphenoxyphosphoryl)oxy]methyl}-1H-1,2,3-triazol-1-yl)piperidine-1-carboxylate (Intermediate 87, 220 mg; 0.3 mmol) was dissolved in anhydrous DMF (3 ml). NaCN (71 mg; 1.4 mmol; 4.8 equiv.) was added in a single portion. The reaction mixture was heated to 60° C. for 4 hours and monitored by LC/MS. The reaction was diluted with EtOAc and washed with buffer (pH=7), brine and then dried over Na₂SO₄. Filter and concentrated to a solid residue. Purified by flash column chromatography to give 103 mg of the title compound in 68% yield. LC/MS (ES) (M+H)⁺: 516, 518 for C₂₃H₂₃Cl₂N₇O₃.

### Intermediate 89

### Benzyl (3S,4R)-4-{[(3,4-dichloro-5-methyl-1H-pyrrol-2-yl)carbonyl]amino}-3-[4-(fluoromethyl)-1H-1,2,3-triazol-1-yl]piperidine-1-carboxylate

Benzyl (3S,4R)-4-{[(3,4-dichloro-5-methyl-1H-pyrrol-2-yl)carbonyl]amino}-3-[4-(hydroxymethyl)-1H-1,2,3-triazol-1-yl]piperidine-1-carboxylate (Intermediate 86, 64 mg; 0.13 mmol) was dissolved in anhydrous DCM (1 ml) and cooled to −78° C. DAST (21 mg; 1.05 equiv.) was added dropwise. The reaction was warmed to 0° C. and stirred for 1 hour. Monitored the reaction by LC/MS. More DAST (63 mg; 3 equiv.) was added to drive the reaction to completion. Quenched the reaction with sat. NaHCO₃. The phases were separated and the organic was dried over Na₂SO₄. The product was purified by flash column chromatography 80% EtOAc/hexanes. Isolation gave 17 mg of the title compound in 26% yield. LC/MS (ES⁺) (M+H)⁺: 509, 511 for C₂₂H₂₃Cl₂FN₆O₃.

### Intermediate 90

### Benzyl (3S,4R)-4-{[(3,4-dichloro-5-methyl-1H-pyrrol-2-yl)carbonyl]amino}-3-[4-(methoxymethyl)-1H-1,2,3-triazol-1-yl]piperidine-1-carboxylate

Benzyl (3S,4R)-4-{[(3,4-dichloro-5-methyl-1H-pyrrol-2-yl)carbonyl]amino}-3-[4-(hydroxymethyl)-1H-1,2,3-triazol-1-yl]piperidine-1-carboxylate (Intermediate 86, 200 mg; 0.4 mmol) was added to a suspension of NaH (78 mg; 2 mmol; 5 equiv.) in THF. MeI (56 mg; 1 equiv.) was added and the reaction was allowed to stir overnight. Quenched with water and extracted with DCM. The organic layer was dried over Na₂SO₄, filtered and concentrated. Purified by flash column chromatography (70% EtOAc/hexanes) to give 85 mg of the title compound in 41% yield. LC/MS (ES⁺) (M+H)⁺: 521, 523 for C₂₃H₂₆Cl₂N₆O₄.

### Intermediate 91

### 3,4-Dichloro-5-methyl-N-[(3S,4R)-3-(1H-1,2,3-triazol-1-yl)piperidin-4-yl]-1H-pyrrole-2-carboxamide hydrobromide

Benzyl (3S,4R)-4-{[(3,4-dichloro-5-methyl-1H-pyrrol-2-yl)carbonyl]amino}-3-(1H-1,2,3-triazol-1-yl)piperidine-1-carboxylate (Intermediate 80, 0.66 mmol) was dissolved in HBr/HOAc (2 ml). The reaction was stirred at room temperature for 2 hours. The acid was removed by rotary evaporation. The solid residue was azeotroped with methanol (×5). No further purification. LC/MS (ES⁺) (M+H)⁺: 343, 345 for C₁₃H₁₆Cl₂N₆O.

### Intermediates 92-98

The following compounds were prepared according to the procedure described above for Intermediate 91 with the starting material shown.

| Int | Compound | Data | SM |
|---|---|---|---|
| 92 | N-[(3S,4R)-3-Azido-4-piperidyl]-3,4- | LC/MS (ES⁺) | Intermediate 79 |
| | dichloro-5-methyl-1H-pyrrole-2-carboxamide | (M + H)⁺: 317, 319 | |
| | hydrobromide | for C₁₁H₁₄Cl₂N₆O | |
| 93 | 3,4-Dichloro-5-methyl-N-[(3S,4R)-3-(4- | LC/MS (ES⁺) | Intermediate 85 |
| | methyl-1H-1,2,3-triazol-1-yl)-4-piperidyl]- | (M + H)⁺: 357, 359 | |
| | 1H-pyrrole-2-carboxamide hydrobromide | for C₁₄H₁₈Cl₂N₆O | |
| 94 | 3,4-Dichloro-N-[(3S,4R)-3-(4-chloro-1H- | LC/MS (ES⁺) | Intermediate 81 |
| | 1,2,3-triazol-1-yl)-4-piperidyl]-5-methyl-1H- | (M + H)⁺: 377, 379 | |
| | pyrrole-2-carboxamide hydrobromide | for C₁₃H₁₅Cl₃N₆O | |
| 95 | N-[(3S,4R)-3-(4-Bromo-1H-1,2,3-triazol-1- | LC/MS (ES⁺) | Intermediate 82 |
| | yl)-4-piperidyl]-3,4-dichloro-5-methyl-1H- | (M + H)⁺: 421, 423, | |
| | pyrrole-2-carboxamide hydrobromide | 425 for | |
| | | C₁₃H₁₅BrCl₂N₆O | |
| 96 | 3,4-Dichloro-N-[(3S,4R)-3-[4- | LC/MS (ES⁺) | Intermediate 86 |
| | (hydroxymethyl)-1H-1,2,3-triazol-1-yl]-4- | (M + H)⁺: 373, 375 | |
| | piperidyl]-5-methyl-1H-pyrrole-2- | for C₁₄H₁₈Cl₂N₆O₂ | |
| | carboxamide hydrobromide | | |
| 97 | 3,4-Dichloro-N-[(3S,4R)-3-[4-(fluoromethyl)- | LC/MS (ES⁺) | Intermediate 89 |
| | 1H-1,2,3-triazol-1-yl]-4-piperidyl]-5-methyl- | (M + H)⁺: 375, 377 | |
| | 1H-pyrrole-2-carboxamide hydrobromide | for C₁₄H₁₇Cl₂FN₆O | |
| 98 | 3,4-Dichloro-N-[(3S,4R)-3-[4- | LC/MS (ES⁺) | Intermediate 90 |
| | (methoxymethyl)-1H-1,2,3-triazol-1-yl]-4- | (M + H)⁺: 387, 389 | |
| | piperidyl]-5-methyl-1H-pyrrole-2- | for C₁₅H₂₀Cl₂N₆O₂ | |
| | carboxamide hydrobromide | | |

### Intermediate 99

### 3,4-Dichloro-N-[(3S,4R)-3-(4-cyano-1H-1,2,3-triazol-1-yl)piperidin-4-yl]-5-methyl-1H-pyrrole-2-carboxamide

Benzyl (3S,4R)-3-(4-cyano-1H-1,2,3-triazol-1-yl)-4-{[(3,4-dichloro-5-methyl-1H-pyrrol-2-yl)carbonyl]amino}piperidine-1-carboxylate (Intermediate 83, 71 mg; 0.14 mmol) was dissolved in DCM (2 ml) and Et₃N (14 μl; 0.7 equiv.). To the mixture was added Et₃SiH (90 μl; 3 equiv.) followed by PdCl₂ (4 mg; 0.3 equiv.). The evolution of gases was observed. The reaction mixture was heated to reflux for 1.5 hours. Monitored the reaction by LC/MS. Quenched with sat. NH₄Cl. Diluted with EtOAc and washed with water then brine. Dried the organic layer over Na₂SO₄, filtered and concentrated to dryness. No further purification. Isolation gave 67.8 mg of the crude reaction product. LC/MS (ES⁺) (M+H)⁺: 368, 370 for C₁₄H₁₅Cl₂N₇O.

### Intermediate 100

### 3,4-Dichloro-N-{(3S,4R)-3-[4-(cyanomethyl)-1H-1,2,3-triazol-1-yl]piperidin-4-yl}-5-methyl-1H-pyrrole-2-carboxamide

Benzyl (3S,4R)-3-[4-(cyanomethyl)-1H-1,2,3-triazol-1-yl]-4-{[(3,4-dichloro-5-methyl-1H-pyrrol-2-yl)carbonyl]amino}piperidine-1-carboxylate (Intermediate 88, 100 mg; 0.19 mmol) was dissolved in methanol. The pressure vessel was charged with Pd—C and H₂ (50 psi). The reaction mixture was agitated for several days. Stopped the reaction at 60% completion and work-up in the usual manner. Pd—C was filtered off through a pad a diatomaceous earth. The mother liquor was concentrated to dryness. No further purification. Isolation gave 95 mg's of the crude title compound. LC/MS (ES⁺) (M+H)⁺: 382, 384 for C₁₅H₁₇Cl₂N₇O.

### Intermediate 101

### tert-Butyl (3R,4R)-4-[(diphenylmethylene)amino]-3-hydroxypiperidine-1-carboxylate

In a sealed hydrogenation flask, purged with nitrogen, benzyl (3R,4R)-4-amino-3-hydroxypiperidine-1-carboxylate (Intermediate 74; 3 grams; 12 mmol) was dissolved in 50 ml of ethanol. Glacial acetic acid (1.4 ml; 24 mmol; 2 equiv.) and palladium on carbon (10 wt %; 100 mg) were added in a single portion. The flask was evacuated and back filled with hydrogen (50 psi). The slurry was agitated at room temperature for 1 hour. The reaction was complete as judged by TLC (90:10:1 CHCl₃/methanol/NH₄OH). The palladium was filtered off through a pad of diatomaceous earth and the resultant mother liquor was concentrated to an oil. No further purification.

Crude (3R,4R)-4-aminopiperidin-3-ol (12 mmol) was dissolved in DCM (50 ml), methanol (10 ml) and DIEA (2.3 grams; 3 ml; 18 mmol; 3 equiv.). Boc-OSu (1-[(tert-butoxycarbonyl)oxy]pyrrolidine-2,5-dione) (2.7 grams; 12.6 mmol; 1.05 equiv.) was added in a single portion. The reaction mixture was stirred at room temperature for 2 hours and monitored by TLC (90:10:1 CHCl₃/methanol/NH₄OH). The reaction mixture was concentrated to an oil.

Crude tert-butyl (3R,4R)-4-amino-3-hydroxypiperidine-1-carboxylate (2.6 g; 12 mmol) and benzophenone imine (2.24 g; 12 mmol; 1.0 eq.) were dissolved in anhydrous toluene and heated to reflux for 18 hrs. Monitored the reaction by TLC (30% EtOAc/hexanes with 0.1% triethylamine). The crude reaction was concentrated and purified by flash column chromatography. Isolation gave 2.8 g of the title compound in an overall 62% yield from Intermediate 74. LC/MS (ES) (M+H)⁺: 381 for C₂₃H₂₈N₂O₃.

### Intermediate 102

### (3S,4R)-tert-Butyl-3-azido-4-[(diphenylmethylene)amino]piperidine-1-carboxylate

In a flame-dried flask triphenylphosphine (3.44 g; 13.1 mmol; 2 eq.) was dissolved in anhydrous THF (15 ml) and cooled to 0° C. DIAD (2.64 g; 13.1 mmol; 2 eq.) was slowly added dropwise. Upon addition a white precipitate formed. A THF solution containing tert-butyl (3R,4R)-4-[(diphenylmethylene)amino]-3-hydroxypiperidine-1-carboxylate (Intermediate 101) (2.5 g; 6.5 mmol) was added (amount of THF added was such that the final concentration of alcohol was ca. 0.5-1M). The resultant reaction slurry was stirred at 0° C. for 30 minutes. (PhO)₂PON₃ (3.06 g; 13.1 mmol; 2 eq.) was then added and the reaction was allowed to warm to RT and stirred for 12 hrs. Monitored by LC/MS. The reaction was concentrated and purified by flash column chromatography (0-30% EtOAc/hexanes with 0.1% triethylamine). Isolation gave 2.2 g of the title compound (mixture of benzophenone and eliminated by-product). LC/MS (ES) (M+H)⁺: 406 for C₂₃H₂₇N₅O₂.

### Intermediate 103

### (3S,4R)-tert-Butyl-3-azido-4-{[(3,4-dichloro-5-methyl-1H-pyrrol-2-yl)carbonyl]amino}piperidine-1-carboxylate

(3S,4R)-tert-Butyl-3-azido-4-[(diphenylmethylene)amino]piperidine-1-carboxylate (Intermediate 102) (2.22 g; 5.5 mmol) was dissolved in 10 ml of aqueous THF (5% H₂O). PPTS (1.4 g; 6.0 mmol; 1.1 eq.) was added in a single portion. The initial cloudy solution became clear within minutes. Upon completion (as determined by LC/MS analysis) the reaction was concentrated and azeotropically dried with acetonitrile. No further purification.

3,4-Dichloro-5-methyl-1H-pyrrole-2-carboxylic acid (1.27 g; 6.6 mmol; 1.2 equiv.) and HATU (3.12 g; 8.22 mmol; 1.5 equiv.) were dissolved in NMP (10 ml) and DIEA (2.1 g; 2.7 ml; 16.4 mmol; 3 eq.). The solution was stirred for 30 minutes. An NMP solution containing the crude (3S,4R)-tert-butyl-4-amino-3-azidopiperidine-1-carboxylate (1.3 g; 5.5 mmol) was added in a single portion. Monitored the reaction by LC/MS and TLC (50% EtOAc/Hex)-ninhydrin stain. Diluted the reaction with EtOAc and washed with water (×2), then brine. Dried over Na₂SO₄, filtered and concentrated to an oil. The compound was purified by flash column chromatography (20%-50% EtOAc/Hex). LC/MS (ES) (M−H)⁻: 417, 419 for C₁₆H₂₂Cl₂N₆O₃.

1. A compound of formula (I): 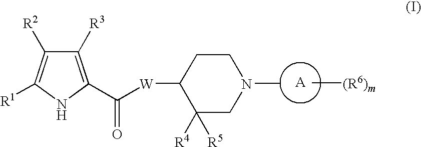
wherein:
R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;
R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;
R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;
W is —O—, —N(R⁷)— or —C(R⁸)(R⁹)—;
Ring A is carbocyclyl or heterocyclyl; wherein if said heterocyclyl contains an —NH— moiety that nitrogen may be optionally substituted by a group selected from R¹⁰;
R⁴ and R⁵ are selected from the following groups: (i) one of R⁴ and R⁵ is hydrogen and the other one is selected from azido, amino or heterocyclyl; or (ii) R⁴ and R⁵ are independently selected from an C₁₋₆alkyl or an C₁₋₆alkoxy group; or (iii) R⁴ and R⁵ together form oxo, R¹¹R¹²N—N═ or R¹³O—N═; or (iv) R⁴ and R⁵ together with the carbon to which they are attached form 3-6 membered carbocyclic or heterocyclic ring wherein said ring may be optionally spiro-fused to a further 3-6 membered carbocyclic or heterocyclic ring; wherein R⁴ and R⁵ in any of groups (i)-(iv) may be optionally substituted on carbon by one or more R¹⁴; wherein if said heterocyclyl in group (i) or heterocyclic ring in group (iv) contains an —NH— moiety that nitrogen may be optionally substituted by a group selected from R¹⁵;
R⁶ is a substituent on carbon and is selected from azido, halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄ alkanoyloxy, N—(C₁₋₄ alkyl)amino, N,N—(C₁₋₄ alkyl)₂amino, C₁₋₄ alkanoylamino, N—(C₁₋₄ alkyl)carbamoyl, N,N—(C₁₋₄ alkyl)₂carbamoyl, N—(C₁₋₄ alkoxy)carbamoyl, N′—(C₁₋₄ alkyl)ureido, N′,N′—(C₁₋₄ alkyl)₂ureido, N—(C₁₋₄ alkyl)-N—(C₁₋₄ alkoxy)carbamoyl, C₁₋₄alkylS(O)ₐ wherein a is 0 to 2, C₁₋₄alkoxycarbonyl, C₁₋₄alkenyloxycarbonyl, C₁₋₄ alkoxycarbonylamino, N—(C₁₋₄ alkyl)sulphamoyl, N,N—(C₁₋₄ alkyl)₂sulphamoyl, C₁₋₄ alkylsulphonylamino, C₁₋₄ alkylsulphonylaminocarbonyl, N′—(C₁₋₄ alkyl)hydrazinocarbonyl, N′,N4C₁₋₄alkyl)₂hydrazinocarbonyl, carbocyclyl-R¹⁶— or heterocyclyl-R¹⁷—; wherein R⁶ may be optionally substituted on carbon by one or more R¹⁸; and wherein if said heterocyclyl contains an —NH— moiety that nitrogen may be optionally substituted by a group selected from R¹⁹;
m is 0-4; wherein the values of R⁶ may be the same or different;
R⁷, R⁸ and R⁹ are independently selected from hydrogen or C₁₋₄alkyl;
R¹¹, R¹² and R¹³ are independently selected from hydrogen, C₁₋₄alkyl, C₁₋₄alkanoyl, C₁₋₄ alkylsulphonyl, C₁₋₄ alkoxycarbonyl, carbamoyl, N—(C₁₋₄ alkyl)carbamoyl, N,N—(C₁₋₄alkyl)carbamoyl, benzyl, benzyloxycarbonyl, benzoyl and phenylsulphonyl; or R¹¹ and R¹² together with the nitrogen to which they are attached form a heterocyclic group; wherein R¹¹, R¹² and R¹³ may be independently optionally substituted on carbon by one or more R²⁰; and wherein if said heterocyclyl contains an —NH— moiety that nitrogen may be optionally substituted by a group selected from R²¹;
R¹⁴ and R¹⁸ are independently selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂amino, C₁₋₄ alkanoylamino, N—(C₁₋₄ alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄ alkyl)sulphamoyl, N,N—(C₁₋₄ alkyl)₂sulphamoyl, C₁₋₄ alkylsulphonylamino, C₁₋₄alkoxycarbonylamino, carbocyclyl-R²²— or heterocyclyl-R²³—; or two R¹⁴ or two R¹⁸ may together form methylene; wherein R¹⁴ and R¹⁸ may be independently optionally substituted on carbon by one or more R²⁴; and wherein if said heterocyclyl contains an —NH— moiety that nitrogen may be optionally substituted by a group selected from R²⁵;
R¹⁰, R¹⁵, R¹⁹, R²¹ and R²⁵ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl; wherein R¹⁰, R¹⁵, R¹⁹, R²¹ and R²⁵ may be independently optionally substituted on carbon by one or more R³¹;
R¹⁶, R¹⁷, R²² and R²³ are independently selected from a direct bond, —O—, —N(R²⁶)—, —C(O)—, —N(R²⁷)C(O)—, —C(O)N(R²⁸)—, —S(O)ₚ—, —SO₂N(R²⁹)— or) —N(R³⁰)SO₂—; wherein R²⁶, R²⁷, R²⁸, R²⁹ and R³⁰ are independently selected from hydrogen or C₁₋₄alkyl and p is 0-2;
R²⁰, R²⁴ and R³¹ are independently selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof;
with the proviso that said compound is not:
cis(±)-methyl 2-(3-azido-4-{[(3,4-dichloro-5-methyl-1H-pyrrol-2-yl)carbonyl]amino}piperidin-1-yl)-1,3-thiazole-5-carboxylate; or
cis(±)-2-(3-azido-4-{[(3,4-dichloro-5-methyl-1H-pyrrol-2-yl)carbonyl]amino}piperidin-1-yl)-1,3-thiazole-5-carboxylic acid.

2-17. (canceled)

18. A compound as claimed in claim 1, or a pharmaceutically acceptable salt thereof, wherein R¹ is C₁₋₄alkyl.

19. A compound as claimed in claim 1, or a pharmaceutically acceptable salt thereof, wherein R² is halo.

20. A compound as claimed in claim 1, or a pharmaceutically acceptable salt thereof, wherein R³ is halo.

21. A compound as claimed in claim 1, or a pharmaceutically acceptable salt thereof, wherein W is —N(R⁷)—; where R⁷ is hydrogen.

22. A compound as claimed in claim 1, or a pharmaceutically acceptable salt thereof, wherein Ring A is thiazolyl, benzothiazolyl or pyridyl.

23. A compound as claimed in claim 1, or a pharmaceutically acceptable salt thereof, wherein R⁴ and R⁵ are selected from the following groups: (i) one of R⁴ and R⁵ is hydrogen and the other one is selected from azido, amino or heterocyclyl; or (ii) R⁴ and R⁵ are independently selected from a C₁₋₆alkoxy group; or (iii) R⁴ and R⁵ together form R¹³O—N═; or (iv) R⁴ and R⁵ together with the carbon to which they are attached form a 3-6 membered heterocyclic ring wherein said ring may be optionally spiro-fused to a further 3-6 membered carbocyclic ring; wherein R⁴ and R⁵ in any of groups (i)-(iv) may be optionally substituted on carbon by one or more R¹⁴; wherein
R¹³ is C₁₋₄alkyl;
R¹⁴ is selected from halo, cyano, C₁₋₄alkyl or C₁₋₄alkoxy; or two R¹⁴ may together form methylene; wherein R¹⁴ and R¹⁸ may be independently optionally substituted on carbon by one or more R²⁴;
R²⁴ is selected from halo, cyano, hydroxy and methoxy.

24. A compound as claimed in claim 1, or a pharmaceutically acceptable salt thereof, wherein R⁶ is a substituent on carbon and is selected from carboxy, carbamoyl, C₁₋₄ alkanoyl, N—(C₁₋₄ alkyl)carbamoyl, N—(C₁₋₄ alkoxy)carbamoyl, C₁₋₄ alkoxycarbonyl, C₁₋₄alkenyloxycarbonyl, carbocyclyl-R¹⁶— or heterocyclyl-R¹⁷—; wherein R⁶ may be optionally substituted on carbon by one or more R¹⁸; and wherein if said heterocyclyl contains an —NH—moiety that nitrogen may be optionally substituted by a group selected from R¹⁹;
R¹⁶ and R¹⁷ are independently selected from a direct bond and —N(R²⁷)C(O)—; wherein R²⁷ is hydrogen;
R¹⁸ is C₁₋₄alkoxy;
R¹⁹ is selected from C₁₋₄alkyl; wherein R¹⁹ may be independently optionally substituted on carbon by one or more R³¹; and
R³¹ is methoxy.

25. A compound as claimed claim 1, or a pharmaceutically acceptable salt thereof, wherein m is 1 or 2; wherein the values of R⁶ may be the same or different.

26. A compound of formula (I): 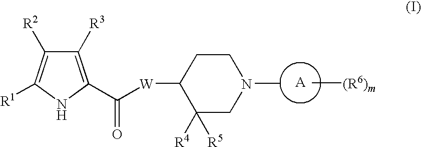
wherein:
R¹ is methyl;
R² is chloro;
R³ is chloro;
W is —N(R⁷)—; where R⁷ is hydrogen;
Ring A is thiazol-2-yl, benzothiazol-2-yl or pyrid-2-yl;
R⁴ and R⁵ are selected from the following groups: (i) one of R⁴ and R⁵ is hydrogen and the other one is selected from azido, amino, imidazol-1-yl, 1,2,3-triazol-1-yl, 4-methyl-1,2,3-triazol-1-yl, 4-cyano-1,2,3-triazol-1-yl, 4-hydroxymethyl-1,2,3-triazol-1-yl, 4-cyanomethyl-1,2,3-triazol-1-yl, 4-fluoromethyl-1,2,3-triazol-1-yl, 4-methoxymethyl-1,2,3-triazol-1-yl, 4-chloro-1,2,3-triazol-1-yl, 3-chloro-1,2,4-triazol-1-yl, 4-bromo-1,2,3-triazol-1-yl or 1,2,4-triazol-1-yl; or (ii) R⁴ and R⁵ are both methoxy; or (iii) R⁴ and R⁵ together form MeO—N═; or (iv) R⁴ and R⁵ together with the carbon to which they are attached form 1,3-dioxolanyl, 5-methoxy-1,3-dioxanyl, 5-ethoxy-1,3-dioxanyl, 5-hydroxymethyl-1,3-dioxanyl, 5,5-dimethyl-1,3-dioxanyl, 5,7-dioxaspiro[2.5]octyl, 5-methylene-1,3-dioxanyl or 1,3-dioxanyl;
R⁶ is a substituent on carbon and is selected from carboxy, carbamoyl, formyl, acetyl, methoxycarbonyl, ethoxycarbonyl, isopropoxycarbonyl, N-(methoxy)carbamoyl, N-(2-methoxyethyl)carbamoyl, N-(1,3-dimethoxyprop-2-yl)carbamoyl, N-(cyclopropyl)carbamoyl, N-(1-methoxyprop-2-yl)carbamoyl, N-(1,3-dimethoxy-2-methoxymethylprop-2-yl)carbamoyl, 1-propen-3-yloxycarbonyl, N-(methyl)carbamoyl, 1-methoxymethylimidazol-2-yl, imidazol-2-yl or 1H-1-methyl-1,2,4-triazol-5-yl.
m is 1 or 2; wherein the values of R⁶ may be the same or different;
or a pharmaceutically acceptable salt thereof.

27. A compound selected from the group consisting of:
4-acetyl-2-(10-{[(3,4-dichloro-5-methyl-1H-pyrrol-2-yl)carbonyl]amino}-1,4-dioxa-7-azaspiro[4.5]dec-7-yl)-1,3-thiazole-5-carboxylic acid;
2-(10-{[(3,4-dichloro-5-methyl-1H-pyrrol-2-yl)carbonyl]amino}-1,4-dioxa-7-azaspiro[4.5]dec-7-yl)-4-{[(2-methoxyethyl)amino]carbonyl}-1,3-thiazole-5-carboxylic acid;
4-acetyl-2-(11-{[(3,4-dichloro-5-methyl-1H-pyrrol-2-yl)carbonyl]amino}-1,5-dioxa-8-azaspiro[5.5]undec-8-yl)-1,3-thiazole-5-carboxylic acid;
2-(4-{[(3,4-dichloro-5-methyl-1H-pyrrol-2-yl)carbonyl]amino}-3,3-dimethoxypiperidin-1-yl)-1,3-thiazole-5-carboxylic acid;
2-[(3E)-4-{[(3,4-dichloro-5-methyl-1H-pyrrol-2-yl)carbonyl]amino}-3-(methoxyimino)piperidin-1-yl]-4-(1-methyl-1H-1,2,4-triazol-5-yl)-1,3-thiazole-5-carboxylic acid;
2-[(3E)-4-{[(3,4-dichloro-5-methyl-1H-pyrrol-2-yl)carbonyl]amino}-3-(methoxyimino)piperidin-1-yl]-4-(1H-imidazol-2-yl)-1,3-thiazole-5-carboxylic acid;
2-((rel-3R,6r,11R)-11-{[(3,4-dichloro-5-methyl-1H-pyrrol-2-yl)carbonyl]amino}-3-methoxy-1,5-dioxa-8-azaspiro[5.5]undec-8-yl)-1,3-thiazole-5-carboxylic acid;
2-((rel-3S,6s,11R)-11-{[(3,4-dichloro-5-methyl-1H-pyrrol-2-yl)carbonyl]amino}-3-methoxy-1,5-dioxa-8-azaspiro[5.5]undec-8-yl)-1,3-thiazole-5-carboxylic acid;
2-[(3S,4R)-4-{[(3,4-dichloro-5-methyl-1H-pyrrol-2-yl)carbonyl]amino}-3-(1H-1,2,3-triazol-1-yl)piperidin-1-yl]-5-(ethoxycarbonyl)-1,3-thiazole-4-carboxylic acid;
2-((3S,4R)-3-(4-chloro-1H-1,2,3-triazol-1-yl)-4-{[(3,4-dichloro-5-methyl-1H-pyrrol-2-yl)carbonyl]amino}piperidin-1-yl)-4-{[(2-methoxyethyl)amino]carbonyl}-1,3-thiazole-5-carboxylic acid; or
2-[(3S,4R)-4-{[(3,4-dichloro-5-methyl-1H-pyrrol-2-yl)carbonyl]amino}-3-(1H-1,2,3-triazol-1-yl)piperidin-1-yl]-N-methoxy-1,3-benzothiazole-7-carboxamide;
or a pharmaceutically acceptable salt thereof.

28. A process for preparing a compound of formula (I) as claimed in claim 1, or a pharmaceutically acceptable salt thereof, which comprises:
Process a) for compounds of formula (I) wherein W is —C(R⁸)(R⁹)—; converting a compound of formula (II): 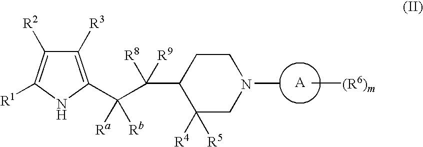
wherein R^{a} is cyano and R^{b} is dimethyamino or diethylamino; or R^{a} and R^{b} are independently selected from C₁₋₄alkylthio; or R^{a} and R^{b} together form 1,3-dithianyl or 1,3-dithiolanyl; into a compound of formula (I); or
Process b) for compounds of formula (I) wherein W is —O—; reacting a compound of formula (III): 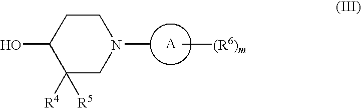
with a compound of formula (IV): 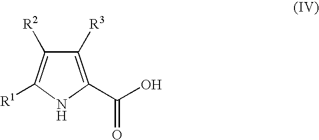
or
Process c) for compounds of formula (I) wherein W is —N(R⁷)—; reacting a compound of formula (V): 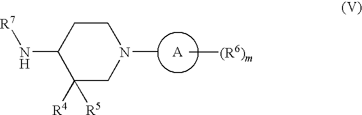
with a compound of formula (IV) or an activated acid derivative thereof; or
Process d) for compounds of formula (I) wherein W is —C(R⁸)(R⁹)—; reacting a compound of formula (VI): 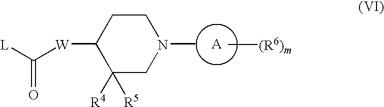
wherein L is a displaceable group; with a compound of formula (VII): 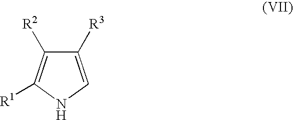
or
Process e) for compounds of formula (I) wherein W is —C(R⁸)(R⁹)—; reacting a compound of formula (VIII): 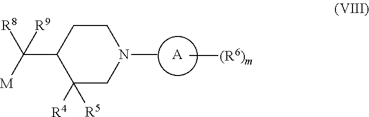
wherein M is an organometallic group; with a compound of formula (IX): 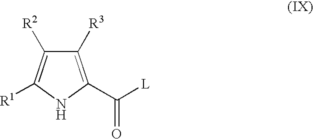
wherein L is a displaceable group; or
Process f) reacting a compound of formula (X): 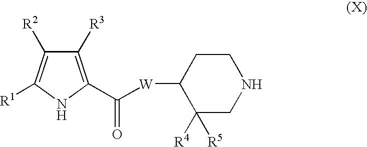
with a compound of formula (XI): 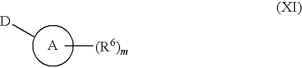
wherein D is a displaceable group; or
Process g) for compounds of formula (I) wherein R⁴ and R⁵ together form R¹¹R¹²N—N═ or R¹³O—N═, by reacting a compound of formula (I) wherein R⁴ and R⁵ together form oxo with an amine of formula R¹¹R¹²N—NH₂ or R¹³O—NH₂; or
Process h) for compounds of formula (I) wherein R⁴ and R⁵ together with the carbon to which they are attached form a heterocyclic ring selected from 1,3-dioxolan-2-yl or 1,3-dioxan-2-yl, by reacting a compound of formula (I) wherein R⁴ and R⁵ together form oxo with 1,2-dihydroxyethane or 1,3-dihydroxypropane;
and thereafter if necessary or desirable:
i) converting a compound of the formula (I) into another compound of the formula (I);
ii) removing any protecting groups;
iii) forming a pharmaceutically acceptable salt.

29. A pharmaceutical composition which comprises a compound as claimed in claim 1, or a pharmaceutically-acceptable salt thereof, and a pharmaceutically-acceptable diluent or carrier.

30. A pharmaceutical composition which comprises a compound as claimed in claim 26, or a pharmaceutically-acceptable salt thereof, and a pharmaceutically-acceptable diluent or carrier.

31. A pharmaceutical composition which comprises a compound as claimed in claim 27, or a pharmaceutically-acceptable salt thereof, and a pharmaceutically-acceptable diluent or carrier.

32. A method for inhibition of bacterial DNA gyrase and/or topoisomerase IV in a warm-blooded animal in need of such treatment which comprises administering to said animal an effective amount of a compound as claimed in claim 1, or a pharmaceutically acceptable salt thereof.

33. A method for inhibition of bacterial DNA gyrase and/or topoisomerase IV in a warm-blooded animal in need of such treatment which comprises administering to said animal an effective amount of a compound as claimed in claim 26, or a pharmaceutically acceptable salt thereof.

34. A method for inhibition of bacterial DNA gyrase and/or topoisomerase IV in a warm-blooded animal in need of such treatment which comprises administering to said animal an effective amount of a compound as claimed in claim 27, or a pharmaceutically acceptable salt thereof.

35. A method of producing an antibacterial effect in a warm-blooded animal in need of such treatment, comprising administering to the animal an effective amount of a compound as claimed in claim 1, or a pharmaceutically acceptable salt thereof.

36. A method of producing an antibacterial effect in a warm-blooded animal in need of such treatment, comprising administering to the animal an effective amount of a compound as claimed in claim 26, or a pharmaceutically acceptable salt thereof.

37. A method of producing an antibacterial effect in a warm-blooded animal in need of such treatment, comprising administering to the animal an effective amount of a compound as claimed in claim 27, or a pharmaceutically acceptable salt thereof.

38. A method of treating a bacterial infection in a warm-blooded animal in need thereof, comprising administering to the animal an effective amount of a compound as claimed in claim 1, or a pharmaceutically acceptable salt thereof.

39. The method of claim 38, wherein the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

40. The method of any one of claim 38, wherein the warm-blooded animal is a human.

41. A method of treating a bacterial infection in a warm-blooded animal in need thereof, comprising administering to the animal an effective amount of a compound as claimed in claim 26, or a pharmaceutically acceptable salt thereof.

42. The method of claim 41, wherein the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

43. The method of any one of claim 41, wherein the warm-blooded animal is a human.

44. A method of treating a bacterial infection in a warm-blooded animal in need thereof, comprising administering to the animal an effective amount of a compound as claimed in claim 27, or a pharmaceutically acceptable salt thereof.

45. The method of claim 44, wherein the bacterial infection is selected from the group consisting of community-acquired pneumoniae, hospital-acquired pneumoniae, skin and skin structure infections, acute exacerbation of chronic bronchitis, acute sinusitis, acute otitis media, catheter-related sepsis, febrile neutropenia, osteomyelitis, endocarditis, urinary tract infections and infections caused by drug resistant bacteria such as Penicillin-resistant Streptococcus pneumoniae, methicillin-resistant Staphylococcus aureus, methicillin-resistant Staphylococcus epidermidis and Vancomycin-Resistant Enterococci.

46. The method of any one of claim 44, wherein the warm-blooded animal is a human.
